SEQUENCE LISTING (3 sequences)

SEQ ID NO: 1 - protein, 8 aa, Artificial sequence
  Cognate peptide
SIINFEKL

SEQ ID NO: 2 - protein, 9 aa, Artificial sequence
  Human gp10025-33 peptide
KVPRNQDWL

SEQ ID NO: 3 - DNA, 20 bp, Artificial sequence
  CpG-1826 phosphorothioate oligonucleotide
tccatgacgttcctgagctt

(19) United States
(12) Patent Application Publication
(10) Pub. No.: US 2018/0008688 A1
(43) Pub. Date: Jan. 11, 2018

(54) METHOD AND COMPOSITIONS FOR ENHANCING IMMUNOTHERAPEUTIC TREATMENT OF A CANCER

(71) Applicant: Augusta University Research Institute, Inc., Augusta, GA (US)

(72) Inventor: David H. Munn, Augusta, GA (US)

(21) Appl. No.: 15/641,427

(22) Filed: Jul. 5, 2017

Related U.S. Application Data
(60) Provisional application No. 62/358,708, filed on Jul. 6, 2016.

(51) Int. Cl. A61K 39/00; C07K 14/47; A61K 39/385

(52) U.S. Cl. CPC A61K 39/0011; A61K 39/385; C07K 14/4748; A61K 39/00; A61K 2039/572

(57) ABSTRACT

Provided are methods and compositions for enhancing treatment of a cancer by administering a therapeutic agent for the treatment of a cancer together with a second agent that elevates the level of protein p53. The second agent generates in the tumor a population of dendritic cells expressing at least one of Batf3, IRF5, CD103, and XCR1. The second therapeutic agent can also suppress an autoimmune response to non-cancerous tissue in the patient if generated by an immunotherapeutic agent. The method can further comprise administering a PTEN phosphatase inhibitor.

### CROSS-REFERENCE TO RELATED APPLICATIONS

This application claims priority to and the benefit of U.S. Provisional Application 62/358,708 titled “METHOD AND COMPOSITIONS FOR ENHANCING IMMUNOTHERAPEUTIC TREATMENT OF A CANCER” filed Jul. 6, 2016, the entire disclosure of which is incorporated herein by reference.

### STATEMENT REGARDING FEDERALLY SPONSORED RESEARCH OR DEVELOPMENT

This invention was made with Government support under contract CA103320 awarded by the National Institutes of Health. The Government has certain rights in this invention.

### SEQUENCE LISTING

The present disclosure includes a sequence listing incorporated herein by reference in its entirety.

### TECHNICAL FIELD

The present disclosure is generally related to methods of enhancing an immunotherapeutic treatment of a cancer by co-administering to a patient an agent that elevates p53 levels and suppresses autoimmune activity. The present disclosure is also generally related to therapeutic compositions that comprise an immunotherapeutic agent and an agent for elevating p53 levels in a recipient patient.

### BACKGROUND

The ability to elicit robust, immunogenic antigen-presentation in tumors is a key determinant of effective cancer immunotherapy (Chen & Mellman (2013) Immunity 39: 1-10). To create a sustained, self-amplifying immune response, it is critical that the tumor's endogenous antigens be effectively cross-presented to the patient's own T cell repertoire (Mittal et al., 2014). Unfortunately, in most tumors the available antigen-presenting cells are profoundly dysfunctional (Tran Janco et al., (2015) J. lmmunol. 194: 2985-2991; Ugel et al., (2015) J. Clin. Invest. 125: 3365-3376).

In mouse tumor models, immunogenic cross-presentation requires a population of dendritic cells dependent on the transcription factor Batf3 (Broz et al., (2014) Cancer Cell, 26: 638-652; Hildner et al., (2008) Science 322: 1097-1100). In tissues, these dendritic cells may express the cell-surface integrin CD103, as well as characteristic markers such as IRF8, XCR1 and CD24 (Salmon et al., (2016) Immunity 44: 924-938). While CD103+ dendritic cells can be present in tumors, they are often limited in number, and many tumors appear to actively exclude them (Broz et al., (2014) Cancer Cell 26: 638-652; Spranger et al., (2015) Nature 523: 231-235). When present, however, CD103+ dendritic cells cross-present tumor antigen (Roberts et al., (2016) Cancer Cell 30:324-336; Salmon et al., (2016) Immunity 44: 924-938), provide pro-inflammatory IL-12 (Zitvogel and Kroemer (2014) Cancer Cell 26: 591-593), attract effector T cells into the tumor (Spranger et al., (2017) Cancer Cell 31: 711-723.e714), and are crucial for anti-tumor responses (Pfirschke et al., (2016) Immunity 44: 343-354; Salmon et al., (2016) Immunity 44: 924-938; Sanchez-Paulete et al., (2015) Cancer Discovery 6: 71-79). The precise human counterpart of these cells is not yet clear, but immunogenic dendritic cells are likely to be important in human tumors as well (Broz et al., (2014) Cancer Cell 26: 638-652; Roberts et al., (2016) Cancer Cell 30: 324-336; Spranger et al., (2017) Cancer Cell 31: 711-723.e714).

Within the tumor microenvironment, however, suppression usually dominates and immunogenic dendritic cells are limited. It is not well understood how immunotherapy can be made to tip this balance, so that the tumor microenvironment now becomes inflammatory and immunogenic. It is now shown that successful conversion to an immunogenic microenvironment critically depends on the maturation of a specific population of CD103− expressing dendritic cells derived from monocyte-lineage cells, and this maturation step is controlled by the transcription factor p53.

### SUMMARY

Briefly described, one aspect of the disclosure encompasses embodiments of a method for enhancing a therapeutic treatment of a cancer, said method comprising the steps of: (a) administering to a patient in need thereof a therapeutic dose of a first therapeutic agent for the treatment of a cancer in said patient; and (b) administering to the patient a therapeutic dose of a second therapeutic agent that elevates the level of protein p53 in said patient.

In some embodiments of this aspect of the disclosure, the first therapeutic agent can be an immunotherapeutic agent or a cytotoxic agent.

In embodiments of this aspect of the disclosure, the second therapeutic agent can generate in a tumor a population of dendritic cells expressing at least one of Batf3, IRF5, CD103, and XCR1.

In some embodiments of this aspect of the disclosure, the second therapeutic agent can suppress an autoimmune response to non-cancerous tissue in the patient generated by the immunotherapeutic agent.

In some embodiments of this aspect of the disclosure, the first therapeutic agent can be an immunotherapeutic agent and the second therapeutic agent can enhance the immunotherapeutic response directed against a tumor in the patient.

In some embodiments of this aspect of the disclosure, the method can further comprise administering to the patient a therapeutic dose of a PTEN phosphatase inhibitor.

In some embodiments of this aspect of the disclosure, the second therapeutic agent can be an inhibitor of a Mouse Double Minute 2 (MDM2) (E3 ubiquitin-protein ligase)-related protein homolog.

In some embodiments of this aspect of the disclosure, the MDM2-related protein inhibitor can be a nutlin, a benzodiazepinedione, a sulphonamide; a chromenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, or stictic acid.

In some embodiments of this aspect of the disclosure, the MDM2-related protein inhibitor can be nutlin-3a.

In some embodiments of this aspect of the disclosure, the first therapeutic agent can be an indoleamine 2,3-dioxygenase (IDO) inhibitor.

In some embodiments of this aspect of the disclosure, the indoleamine 2,3-dioxygenase (IDO) inhibitor can be 1-methyl-D-tryptophan (D1MT), 1-cyclohexyl-2-(5H-irnidazo[5,1-a]isoindol-5-yl)ethanol (GDC919/NLG919), or (E)-4-Amino-N′-(3-chloro-4-fluorophenyl)-N-hydroxy-1,2,5-oxadiazole-3-carboximidamide (INCB024360).

In some embodiments of this aspect of the disclosure, the first therapeutic agent can be an anthracene cytotoxic agent selected from the group consisting of doxorubicin, idarubicin, and mitoxantrone.

In some embodiments of this aspect of the disclosure, the first and the second therapeutic agents can be individually administered to the patient.

In some embodiments of this aspect of the disclosure, the first and the second therapeutic agents can be administered in a single formulation.

In some embodiments of this aspect of the disclosure, the first and the second therapeutic agents and the PTEN phosphatase inhibitor can be individually administered to the patient.

In some embodiments of this aspect of the disclosure, the first and the second therapeutic agents and the PTEN phosphatase inhibitor are administered in a single formulation.

Another aspect of the disclosure encompasses embodiments of a composition comprising a first therapeutic agent for the treatment of a cancer in a recipient patient and a second therapeutic agent that elevates the level of protein p53 in said patient.

In some embodiments of this aspect of the disclosure, the first therapeutic agent can be an immunotherapeutic agent or a cytotoxic agent.

In some embodiments of this aspect of the disclosure, the composition can further comprise a PTEN phosphatase inhibitor.

In some embodiments of this aspect of the disclosure, the composition can further comprise a pharmaceutically acceptable carrier.

In some embodiments of this aspect of the disclosure, the composition can be formulated for delivering to a patient in need thereof an amount of an immunotherapeutic agent effective in generating an immune response directed against a tumor in the recipient patient and an amount of the second therapeutic agent effective in enhancing the immunotherapeutic response directed against a tumor of the patient by generating a population of dendritic cells expressing at least one of Batf3, IRF5, CD103, and XCR1 in the tumor.

In some embodiments of this aspect of the disclosure, the second therapeutic agent can further suppress an autoimmune response to non-cancerous tissue in the patient generated by the immunotherapeutic agent.

In some embodiments of this aspect of the disclosure, the second therapeutic agent can be an inhibitor of a Mouse Double Minute 2 (MDM2) (E3 ubiquitin-protein ligase) MDM2-related protein homolog.

In some embodiments of this aspect of the disclosure, the MDM2-related protein inhibitor can be a nutlin, a benzodiazepinedione, a sulphonamide; a chromenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, or stictic acid.

In some embodiments of this aspect of the disclosure, the MDM2-related protein inhibitor can be nutlin-3a.

In some embodiments of this aspect of the disclosure, the immunotherapeutic agent is an indoleamine 2,3-dioxygenase (IDO) inhibitor.

In some embodiments of this aspect of the disclosure, the indoleamine 2,3-dioxygenase (IDO) inhibitor can be 1-methyl-D-tryptophan (D1MT), -cyclohexyl-2-(5H-imidazo[5,1-a]isoindol-5-yl)ethanol (GDC919/NLG919), or (E)-4-Amino-N′-(3-chloro-4-fluorophenyl)-N-hydroxy-1,2,5-oxadiazole-3-carboximidamide (I NCB024360).

In some embodiments of this aspect of the disclosure, the cytotoxic agent can be an anthracene selected from the group consisting of doxorubicin, idarubicin, and mitoxantrone.

Still another aspect of the disclosure encompasses embodiments of a composition comprising an immunotherapeutic agent effective in generating an immune response directed against a tumor in a recipient patient, a therapeutic agent that elevates the level of protein p53 in a recipient patient, wherein said therapeutic agent is nutlin-3a, and a pharmaceutically acceptable carrier.

In some embodiments of this aspect of the disclosure, the composition can further comprise at least one of an IDO-inhibitor and a cytotoxic agent.

Still another aspect of the disclosure encompasses embodiments of a kit comprising a first therapeutic agent directed against a tumor in a recipient patient, a second therapeutic agent that elevates the level of protein p53 in a recipient patient, and a pharmaceutically acceptable carrier, wherein the first therapeutic agent, the second therapeutic agent, and the pharmaceutically acceptable carrier are packaged individually or in any combination, and instructions for the use of the packaged agents and carrier to prepare an effective dose of each agent for administration individually or in combination to a patient in need thereof.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1D illustrate inflammation in tumors elicits immunogenic dendritic cells expressing markers of both myeloid cells and conventional dendritic cells.

FIG. 1A illustrates B16F10 tumors implanted in PTEN^{Treg}-KO mice or WT controls. The graph shows mean ±SD for 24-48 tumors at each data-point, pooled from multiple independent experiments, *p<0.001. The FACS plots (center) show the representative phenotype of gated CD11c⁺ cells in the tumor (day 20). CD11c was detected with clone HL3, to minimize cross-reactivity with macrophages. Quadrant markers were set based on isotype controls. The scatter plot shows the percentage of Ly6c⁺/CD103⁺ dendritic cells in 9 experiments; ** p<0.01.

FIG. 1B illustrates B16F10 tumors in VVT hosts treated with PTEN-inhibitor (VO-OHpic, 10 mg/kg/d i.p.) plus cyclophosphamide (CTX) at the doses shown. * p<0.001 vs control groups, ** p<0.001 vs all other groups by ANOVA.

FIG. 10 illustrates the phenotype of the Ly6c⁺/CD103⁺ population of CD11c⁺ cells in tumors, 4 days after treatment with VO-OHpic+CTX (150 mg/kg). Each marker is representative of 4-18 experiments each. Upper scatter-plot shows the percentage of Ly6c⁺CD103⁺ dendritic cells in treated versus untreated tumors. Lower scatter-plot shows the expression of each marker on the gated Ly6c⁺/CD103⁺ population, pooled across all treated tumors.

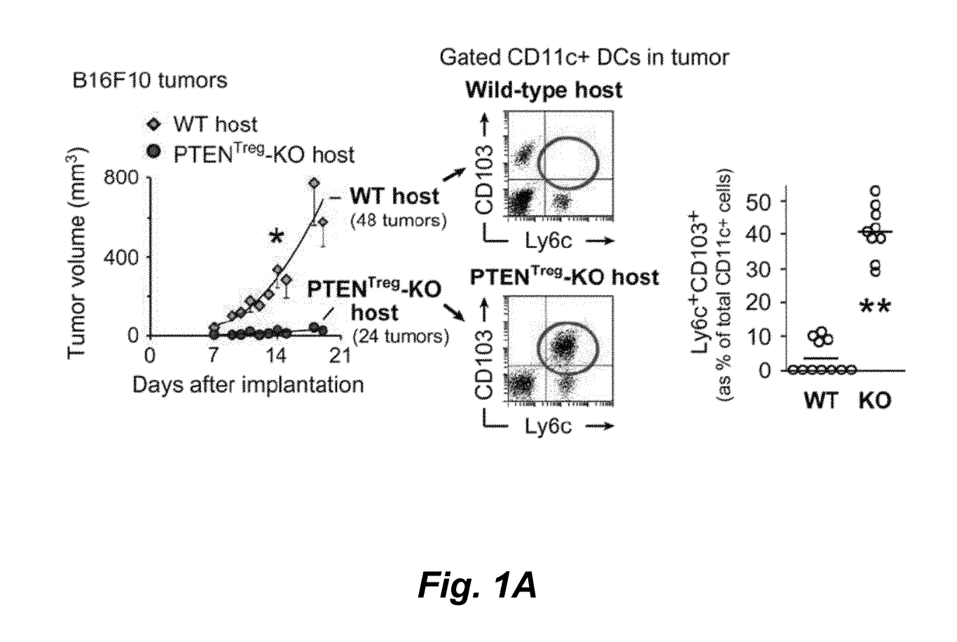

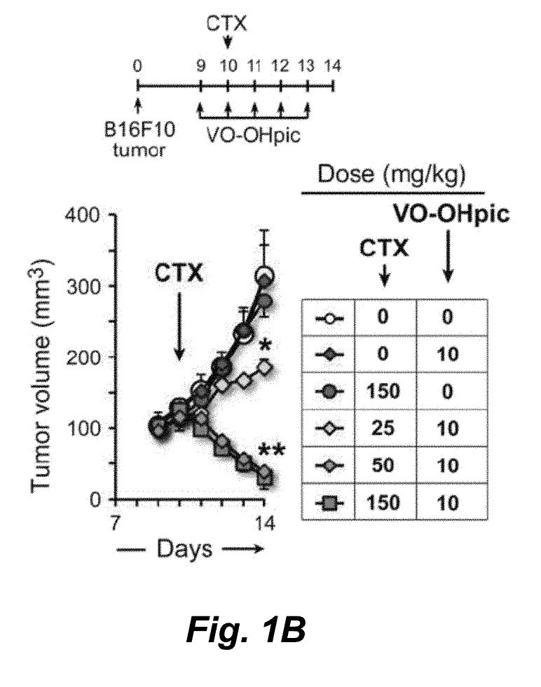

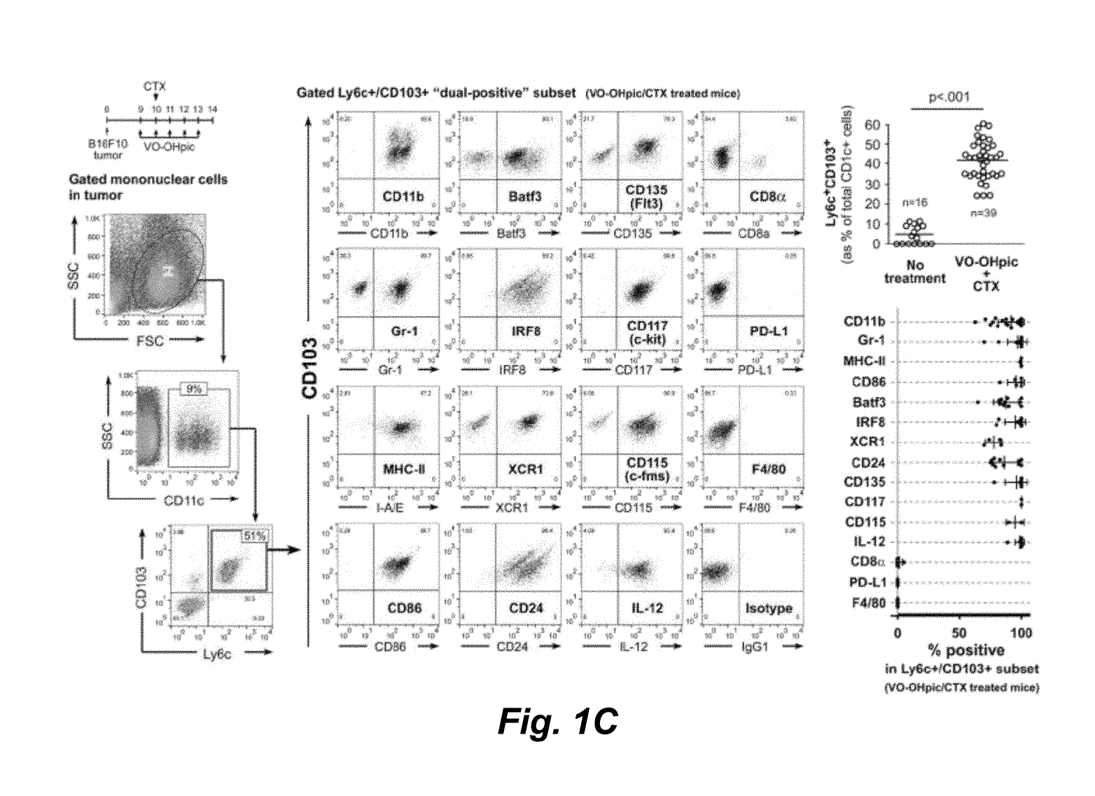

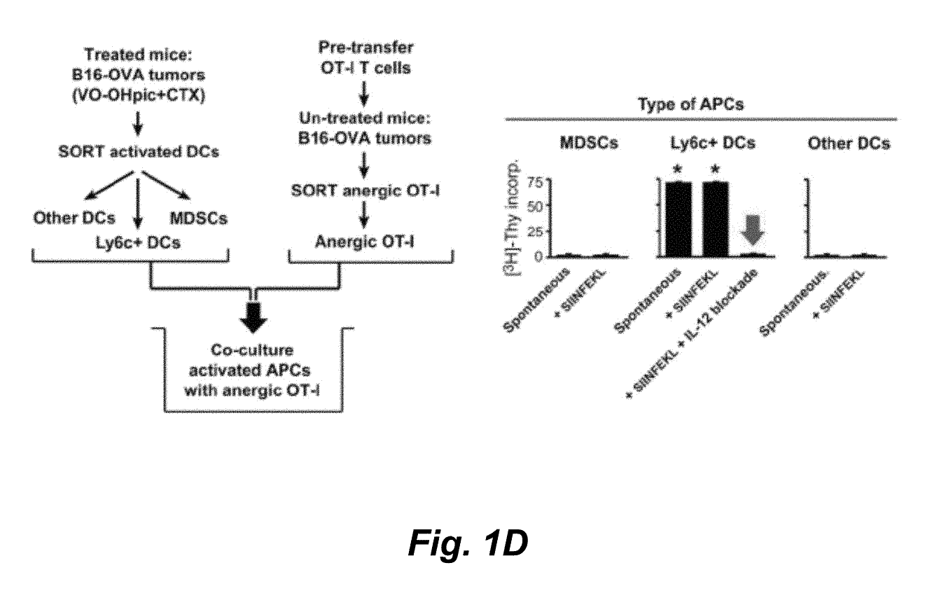

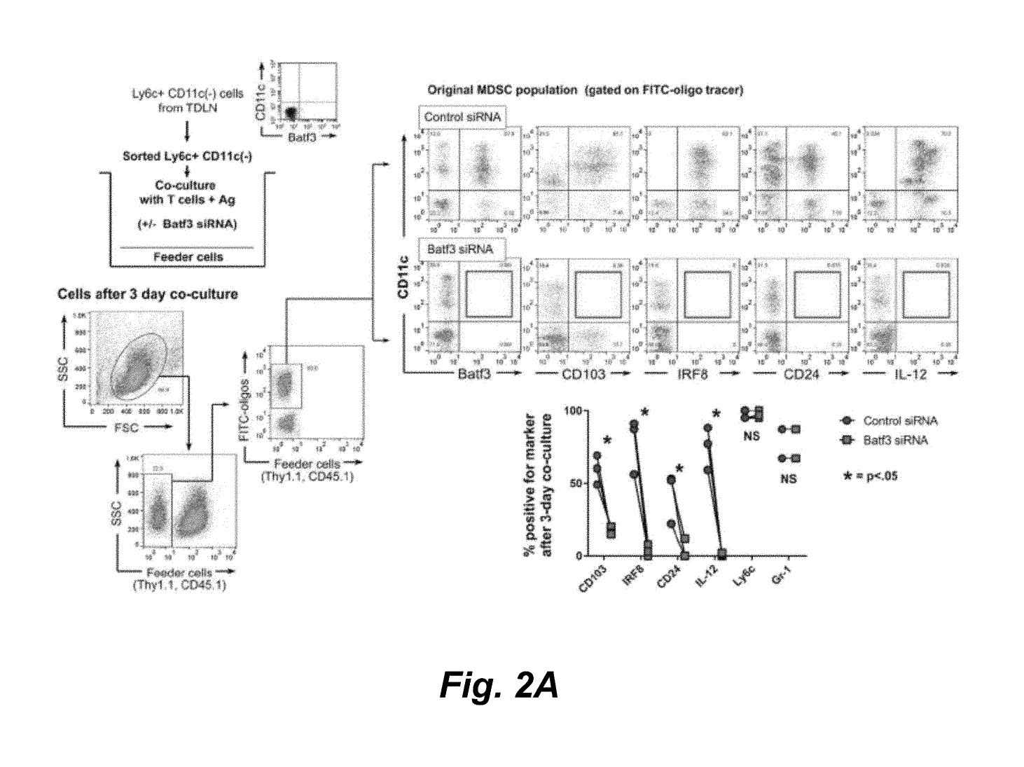

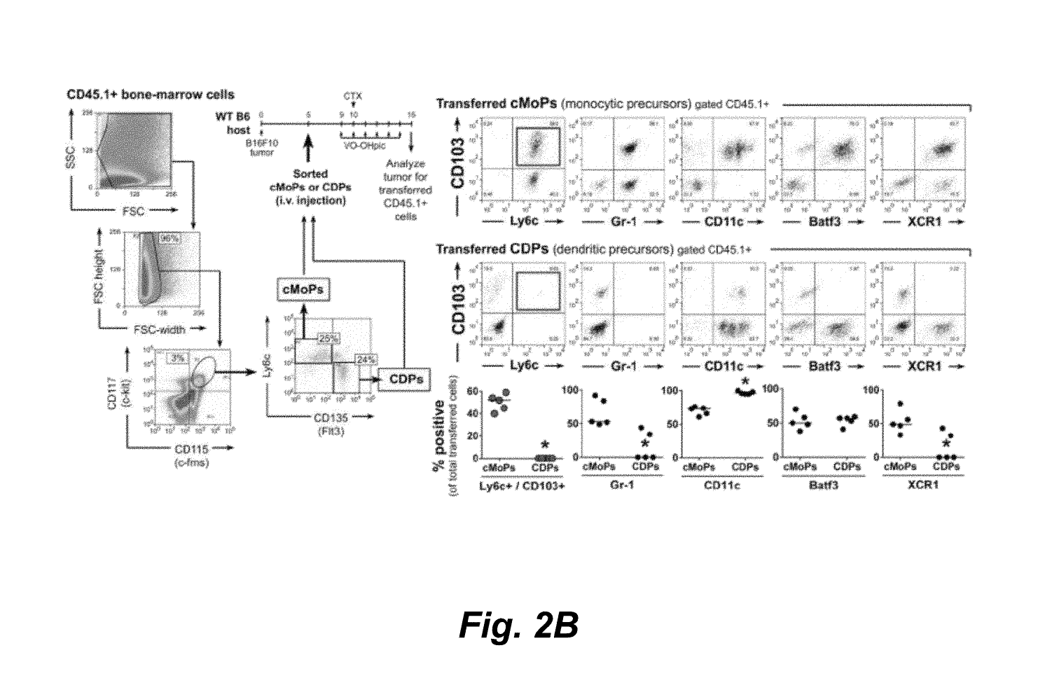

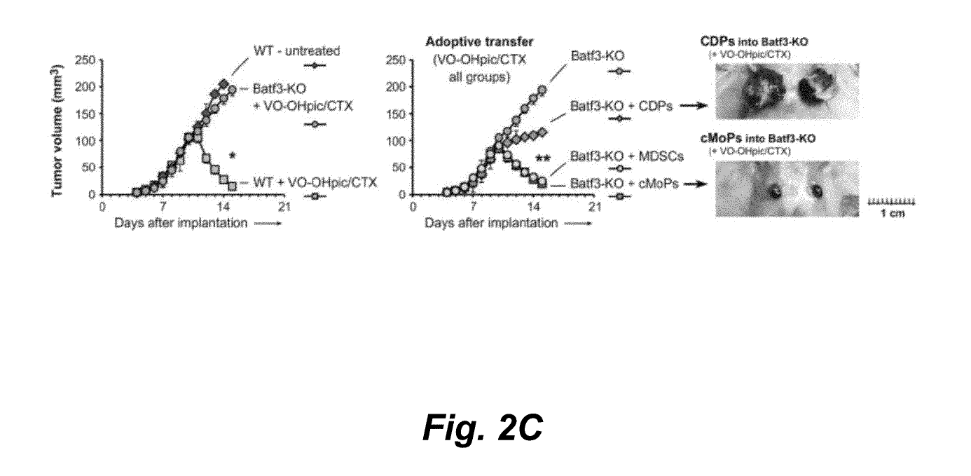

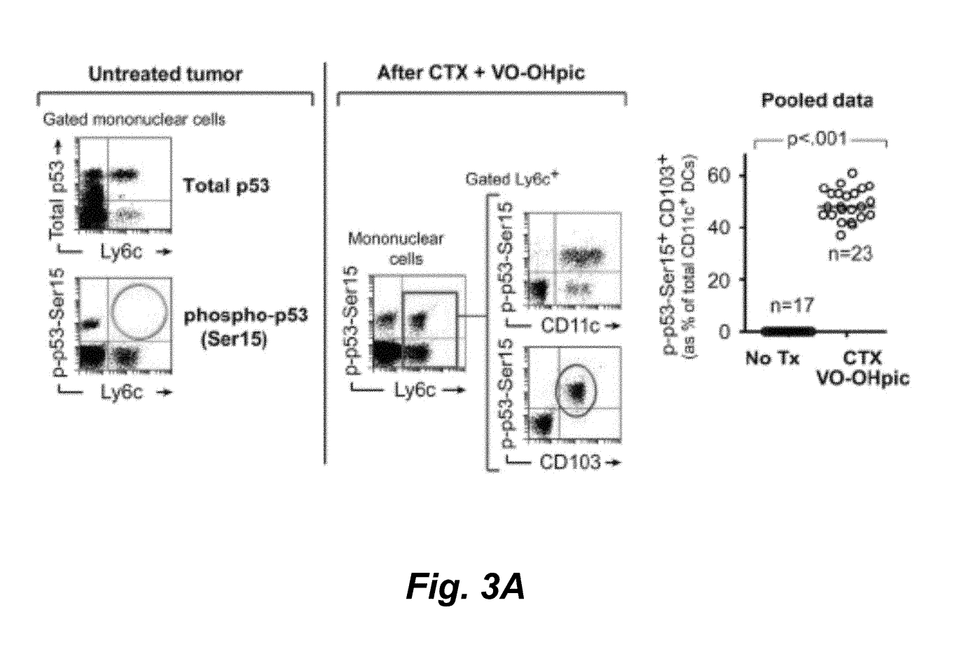

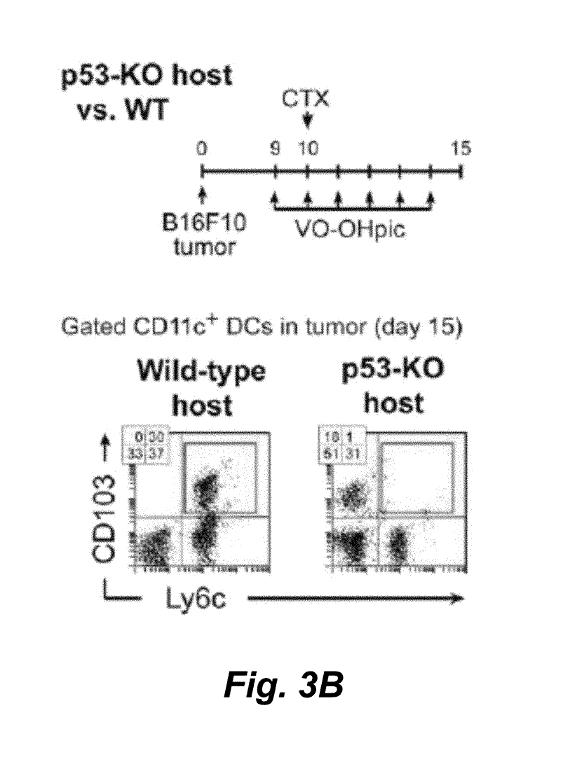

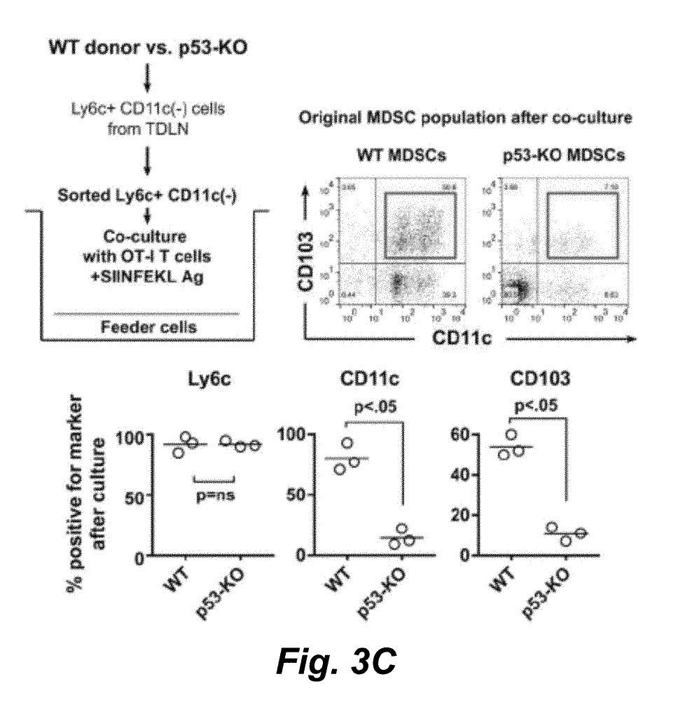

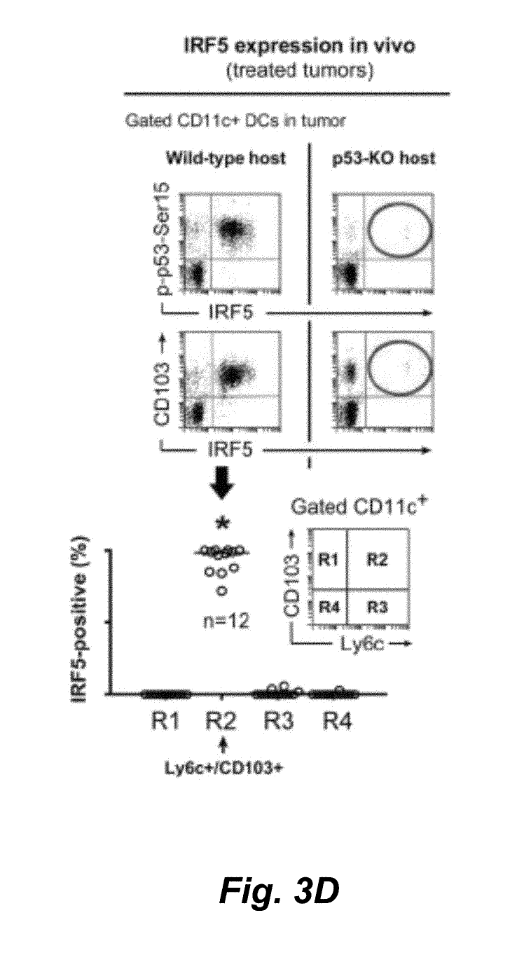

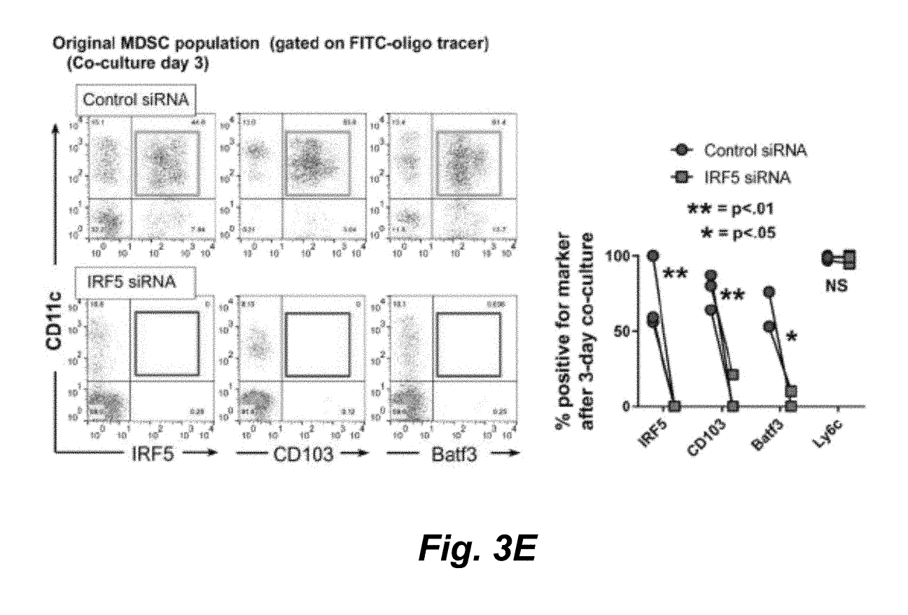

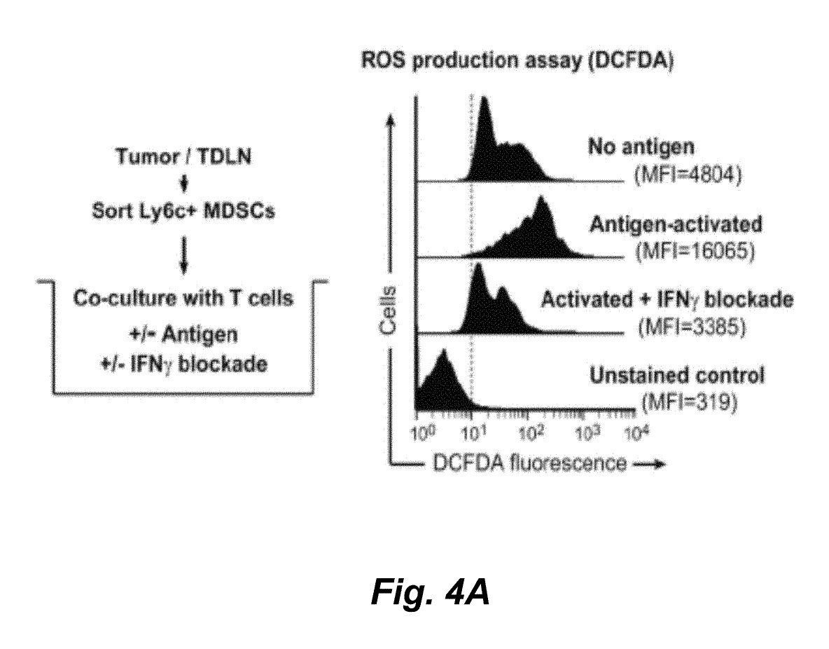

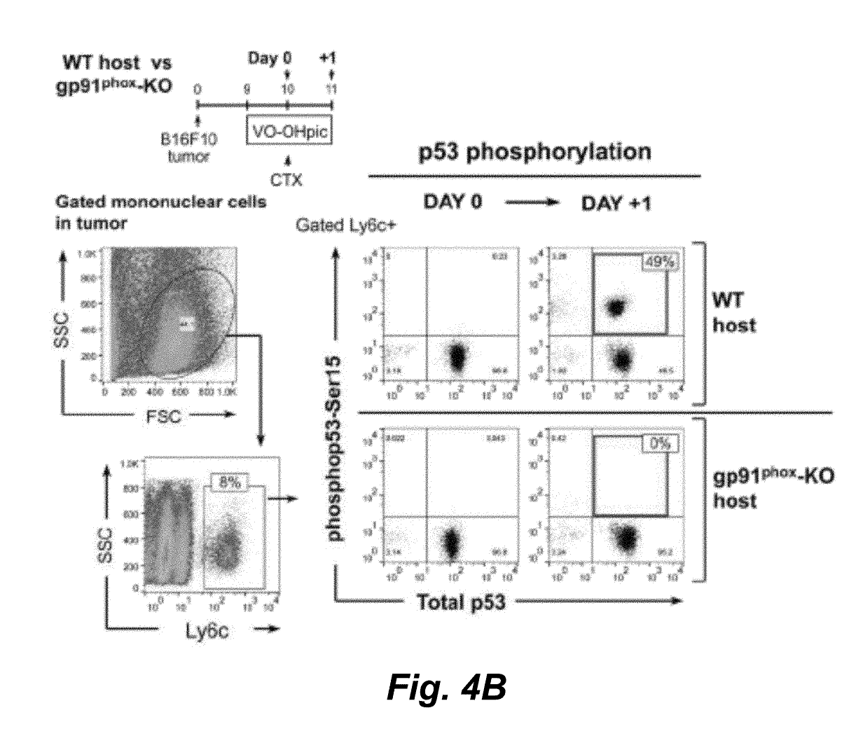

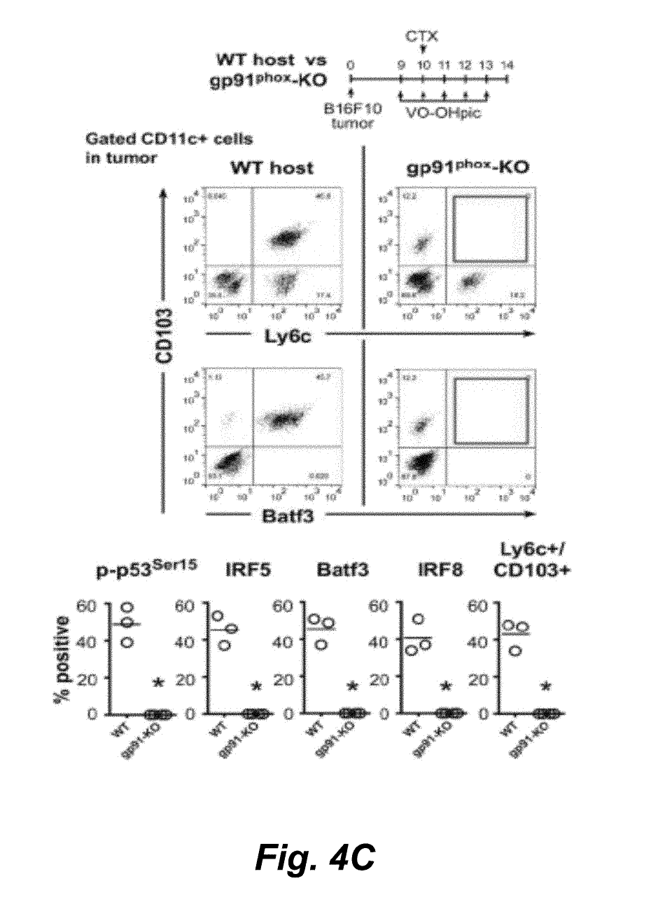

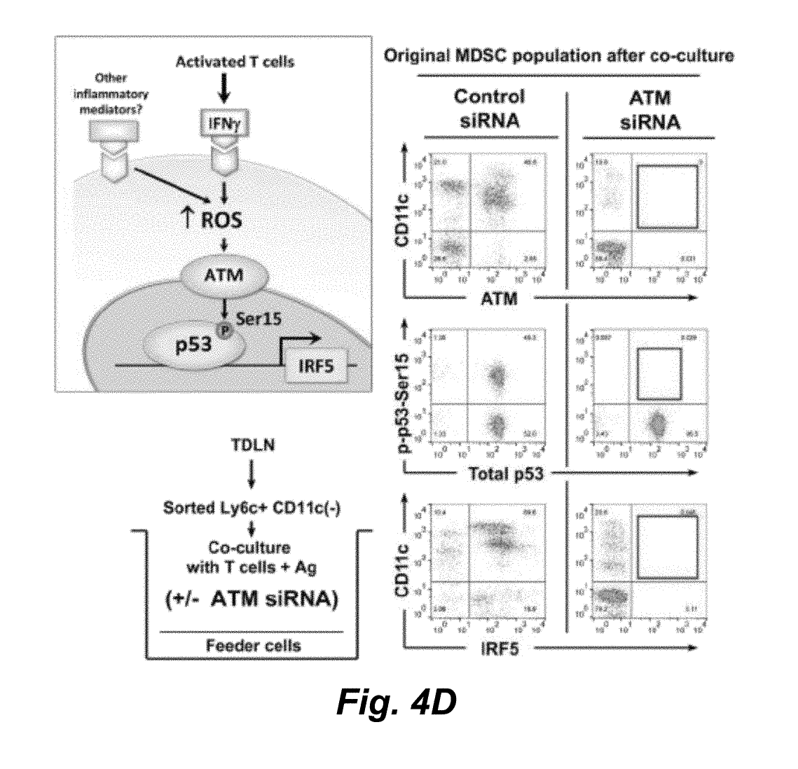

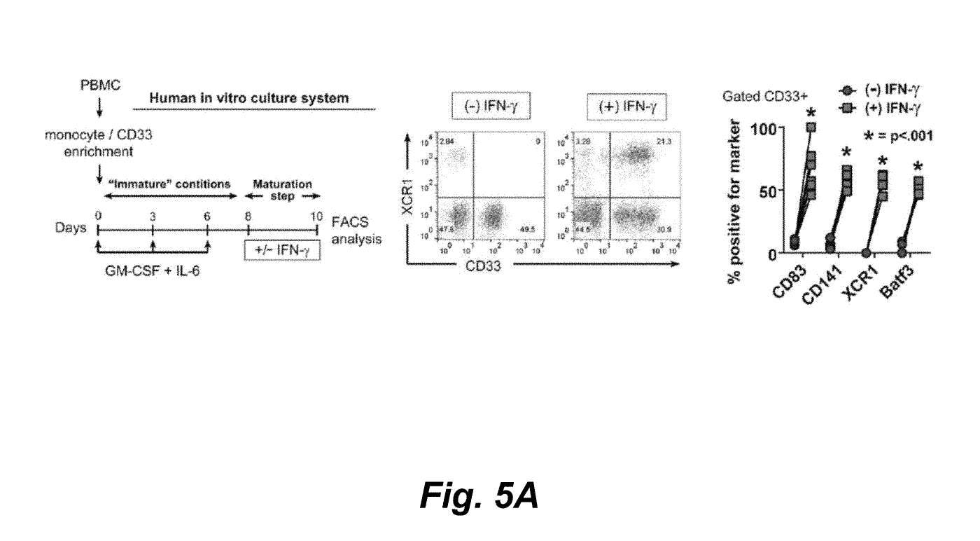

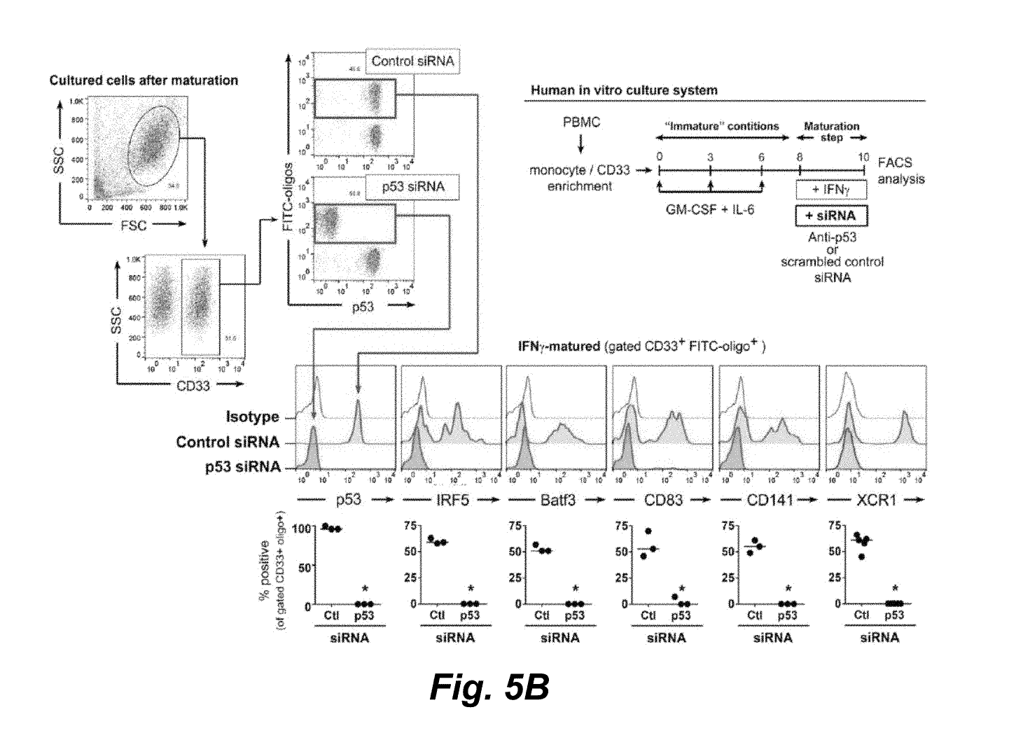

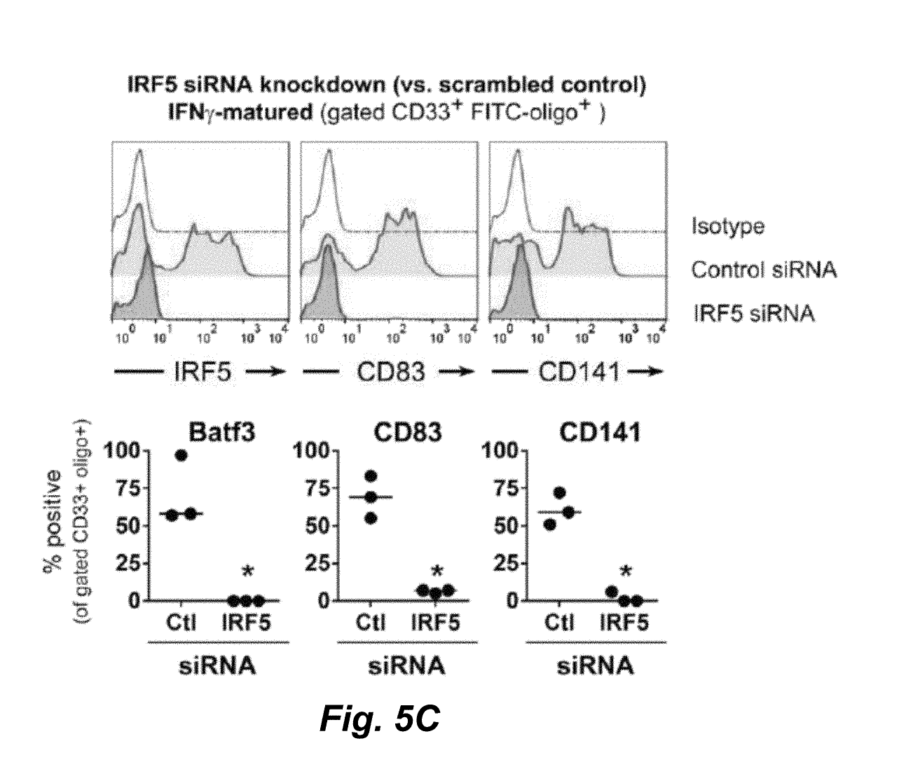

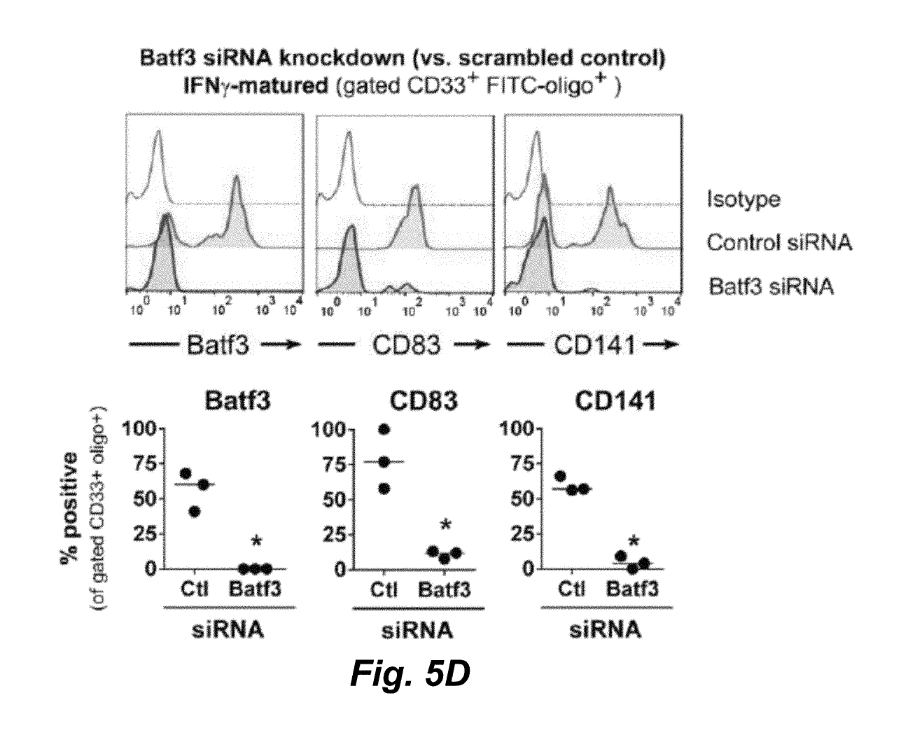

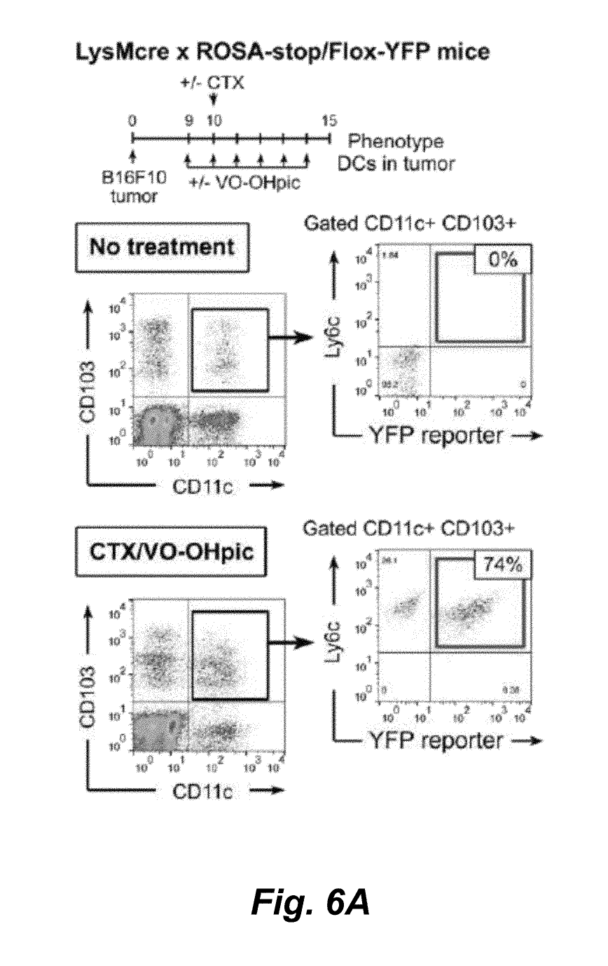

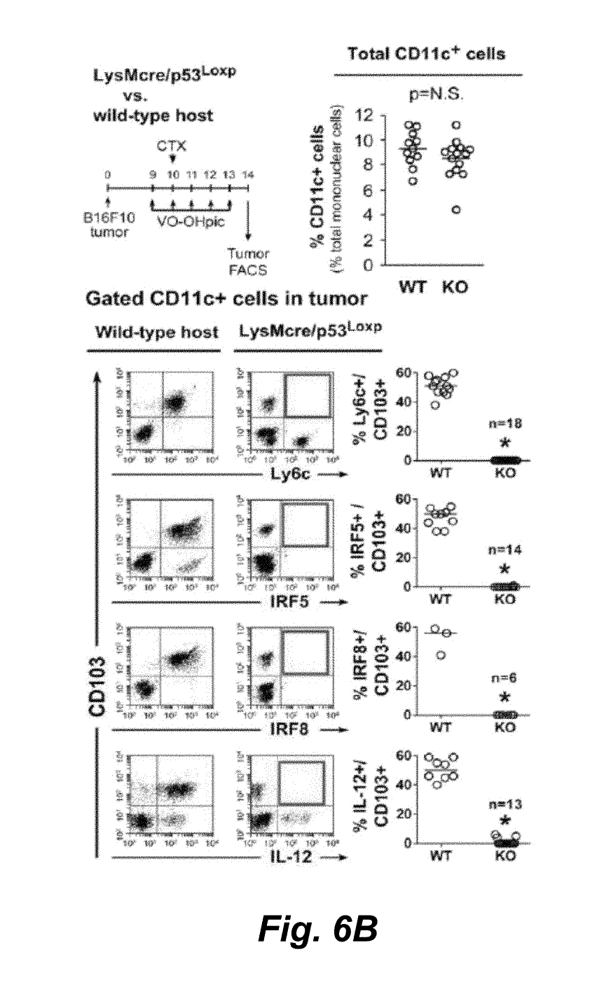

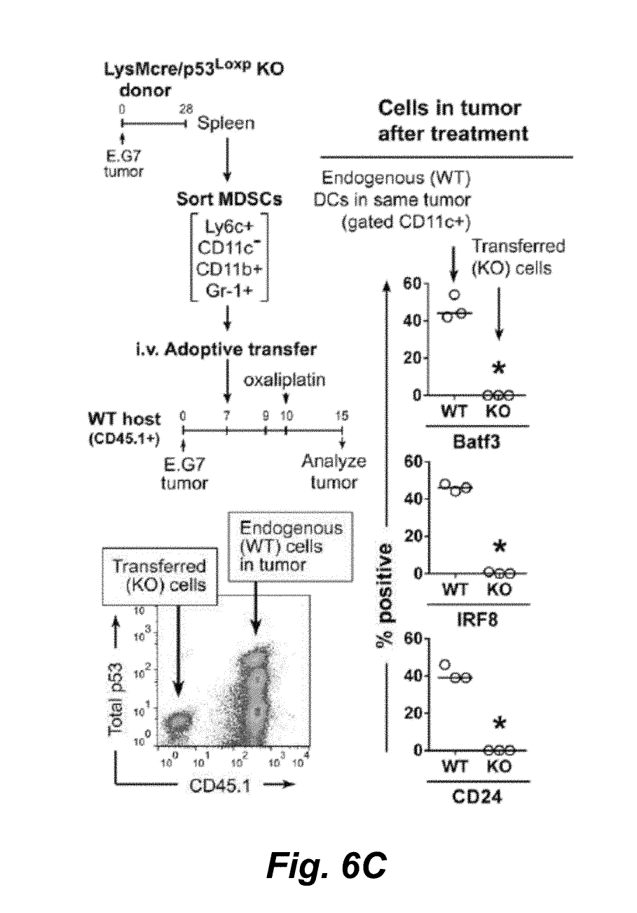

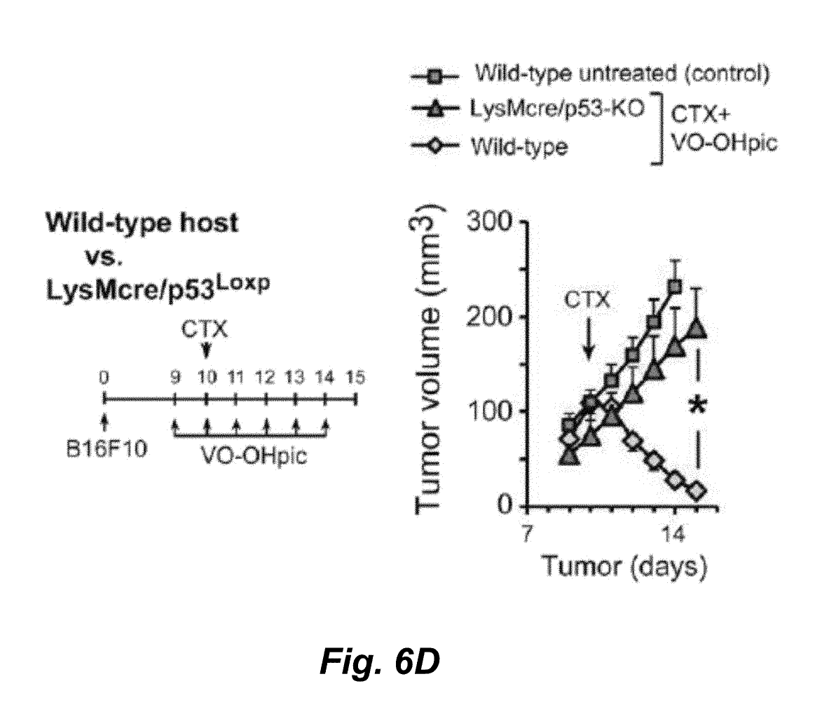

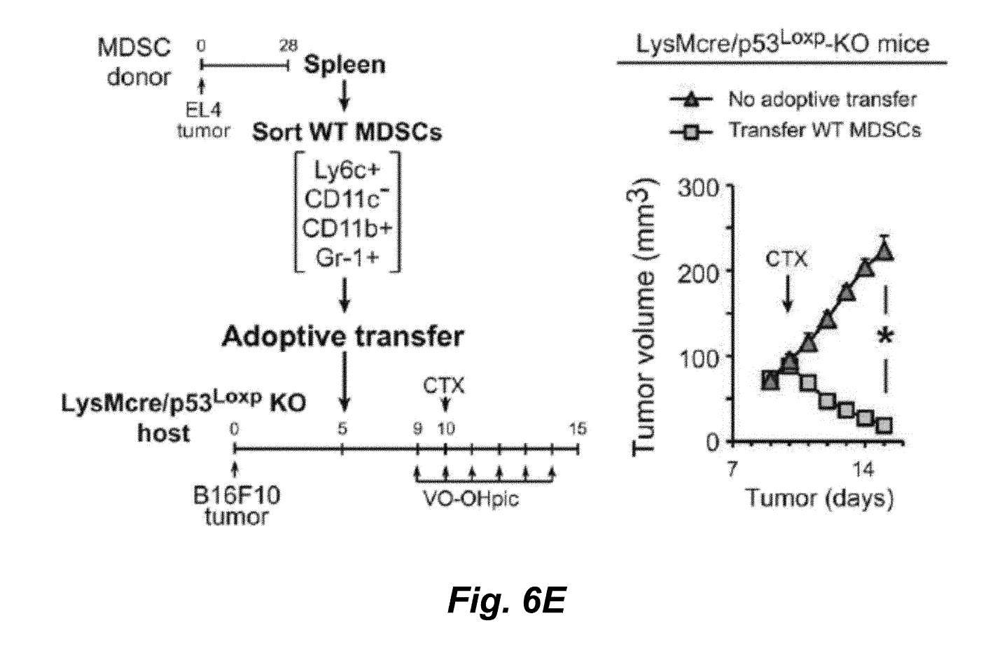

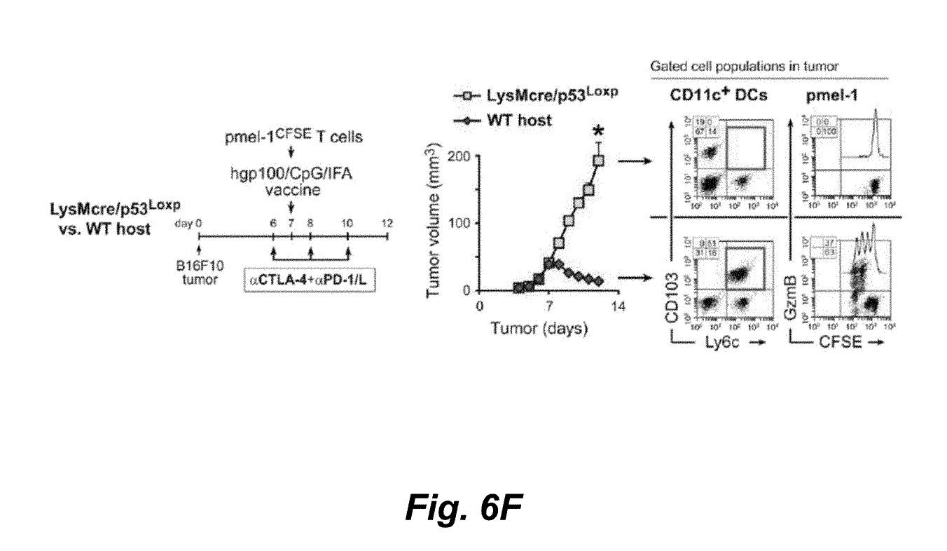

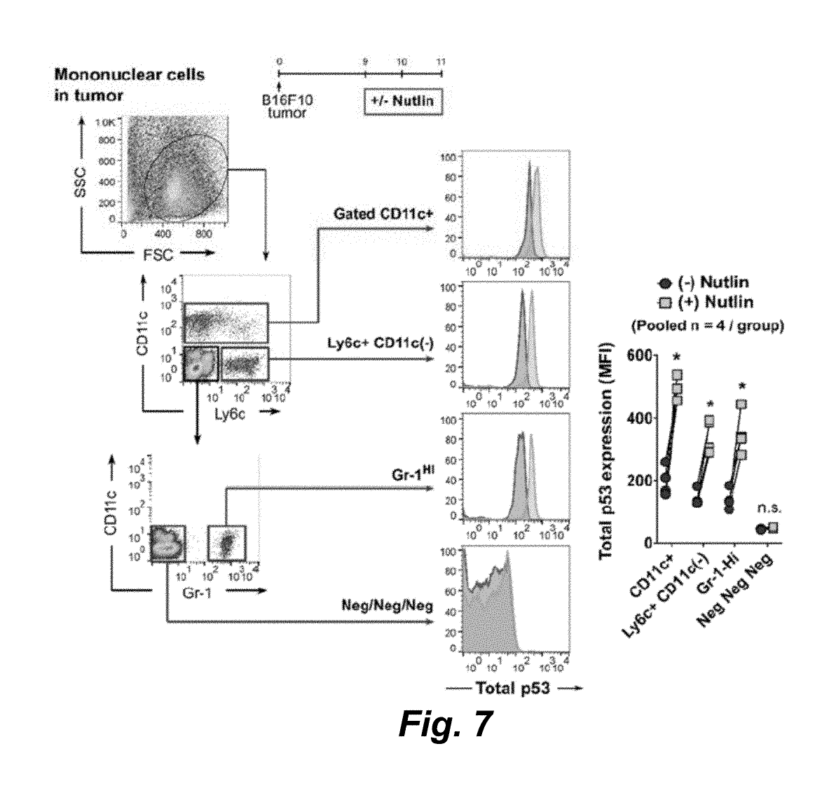

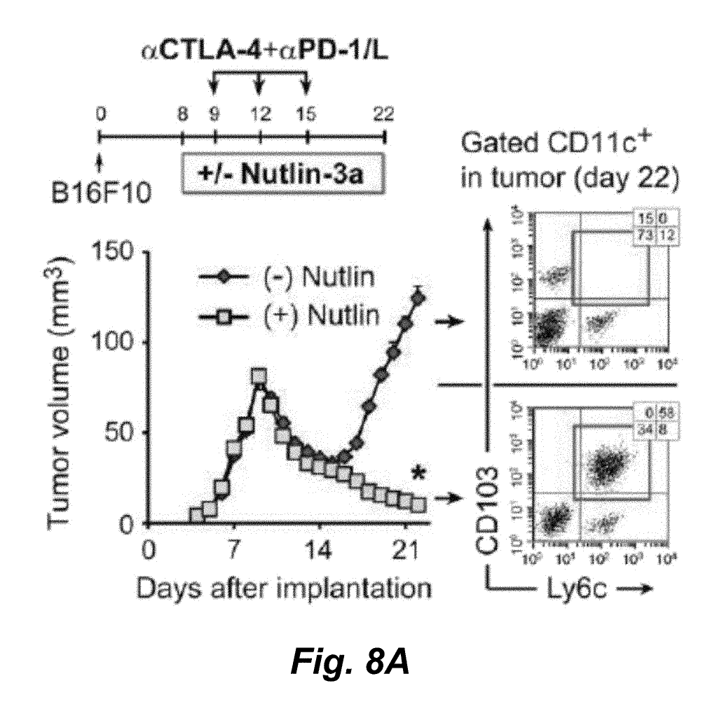

FIG. 1D illustrates in vitro rescue of anergic T cells by Ly6c⁺/CD103⁺ dendritic cells. Anergized OT-I T cells were isolated from tumor-bearing hosts; then co-cultured with activated Ly6c⁺/CD103⁺ dendritic cells, enriched from tumors treated with CTX/VO-OHpic. Wells received either cognate SIINFEKL (SEQ ID NO: 1) peptide, or relied only on antigen acquired by the dendritic cells in vivo (“spontaneous” groups). IL-12 was blocked with anti-IL-12p40 antibody. Representative of 3 experiments. *p<0.01 by ANOVA.

FIGS. 2A-2C illustrate that Ly6c⁺/CD103⁺ dendritic cells can differentiate directly from Ly6c⁺ myeloid precursor cells.

FIG. 2A illustrates Batf3 controls in vitro differentiation of peripheral Ly6c⁺CD11c^{NEG} cells. Ly6c⁺ cells were sorted from TDLNs of B16F10 tumors and co-cultured for 72 h with OT-I T cells, cognate antigen and feeder cells. Cultures received siRNA against Batf3 or scrambled control at the start of culture. Markers are shown gated on cells taking up the FITC-labeled tracer oligonucleotides. Constitutive expression of Ly6c and Gr-1 were unaffected by Batf3 knockdown (scatter-plot), confirming that the siRNA itself was not toxic. Pooled results of 3 identical experiments.

FIG. 2B illustrates bone-marrow cells from normal CD45.1⁺ mice (without tumors) were sorted into monocytic cMoP cells (c-fms/CD115⁺, c-kit/CD117⁺, Ly6c⁺ and Flt3/CD135^{NEG}) or dendritic-lineage CDP cells (CD115⁺ CD117⁺ CD135⁺ Ly6c^{NEG}), as shown. Cells were injected intravenously into C57BL/6 mice with established tumors and treated with CTX/VO-OHpic. Phenotype shows transferred cells in tumors after treatment. Pooled data from 4 experiments. *p<0.01 by ANOVA.

FIG. 2C illustrates the rescue of functional anti-tumor activity in Batf3-deficient mice by adoptive transfer of myeloid precursor cells. Left-hand graph shows inability of control Batf3-KO mice to respond to immune-dependent CTX/VO-OHpic treatment. Right-hand graph shows response after day 5 i.v. transfer of either VVT cMoPs or CDPs (*1×10⁵ each, sorted as in the previous panel); or by Ly6c+CD11c^{NEG} Gr-1+ CD11b⁺ MDSCs (*1×10⁵, sorted from spleens of tumor-bearing donors). Photos show representative tumors on day 15.

FIGS. 3A-3E illustrate that p53 controls differentiation of Ly6c⁺/CD103⁺ dendritic cells in an IRF5-dependent fashion.

FIG. 3A illustrates the expression of total p53 and phosphorylated p53-Ser15 in Ly6c⁺CD103⁺ dendritic cells before and after treatment with CTX/VO-OHpic. Scatter-plot shows pooled data; p-value by unpaired t-test.

FIG. 3B illustrates p53-KO mice or VVT controls bearing B16F10 tumors were treated with CTX/VO-OHpic; plots show Ly6c+/CD103⁺ dendritic cells in tumor on day 15. Representative of 5 experiments.

FIG. 3C illustrates tumors were grown in p53-KO mice or VVT controls. Ly6c⁺CD11c^{NEG} immature myeloid cells were sorted from TDLNs and tested in differentiation assays, as in FIG. 2A. p values by ANOVA.

FIG. 3D illustrates the expression of IRF5 in CD11c⁺ cells from tumors in WT or p53-KO hosts treated with CTX/VO-OHpic. Scatterplots show close concordance of IRF5 with the Ly6c⁺/CD103⁺ subset of dendritic cells. n=12; * p<0.001 by ANOVA.

FIG. 3E illustrates siRNA knock-down of IRF5 (or scrambled control siRNA).

FIGS. 4A-4D illustrate the proximate trigger for p53 activation is the myeloid respiratory burst.

FIG. 4A illustrates in vitro differentiation of Ly6c⁺CD11c^{NEG} cells as in FIG. 2A, with or without anti-IFNy blocking antibody. ROS measured using 2′,7′-dichlorofluorescein diacetate (DCFDA). Representative of 3 experiments.

FIGS. 4B and 4C illustrate tumors implanted in Cybb-deficient (gp91 phox-KO) mice or WT controls and treated with CTX/VO-OHpic.

FIG. 4B illustrates phosphorylation of p53 on Ly6c⁺ cells, 1 day after chemotherapy.

FIG. 4C illustrates dendritic cells analyzed in tumors. Scatterplots show markers on gated CD11c⁺ cells; *p<0.01 by ANOVA.

FIG. 4D illustrates an in vitro differentiation assay with siRNA knockdown of ATM kinase (vs. scrambled control siRNA). Representative of 4 experiments.

FIGS. 5A-5D illustrate that maturation of human myeloid cells into CD141⁺ dendritic cells is dependent on p53 and IRF5.

FIG. 5A illustrates human peripheral blood mononuclear cells cultured with or without recombinant IFNγ added during the final 2 days. FACS plots show a representative example of one maturation marker. Scatter-plots show pooled data from 5-7 experiments for each marker. *=p<0.001 by ANOVA for effect of IFNyγ.

FIGS. 5B-5D illustrate human cells cultured and matured with IFNγ (and incubated with or without an siRNA against the indicated targets or a scrambled control siRNA added during maturation). Histograms show the effect of siRNA knockdown on each marker in one representative experiment. Scatter-plots show 3 pooled experiments; *p<0.01 by ANOVA for effect of siRNA.

FIG. 5B: anti-p53 siRNA: dot-plots show effective knockdown of p53 in cells that were transduced (FITC-labeled tracer oligonucleotides).

FIG. 5C: anti-IRF5 siRNA. FIG. 5D: anti-Batf3 siRNA.

FIGS. 6A-6F illustrate that targeted deletion of p53 in myeloid cells blocks differentiation of the Ly6c⁺/CD103⁺ dendritic cell population and profoundly compromises antitumor activity.

FIG. 6A illustrates LysMcre mice were crossed with the ROSA-stop/Flox-YFP reporter strain. Mice bearing B16F10 tumors were treated with CTX/VO-OHpic or were untreated controls. Mononuclear cells in tumor were gated on the CD11c⁺CD103⁺ population. Expression of YFP vs. Ly6c shown. Representative of 6 experiments.

FIG. 6B illustrates LysMcre mice were crossed with homozygous p53-flox/flox mice to produce LysMcre/p53^{Loxp} mice. B16F10 tumors were implanted in LysMcre/p53^{Loxp} or into WT controls, and treated with CTX/VO-OHpic. Upper scatterplot compares total CD11c⁺ cells in tumors after treatment. FACS plots show phenotype of the CD11c⁺ population, with quantitation of each of the subsets shown. * p<0.001 by ANOVA.

FIG. 6C illustrates E.G7 tumors grown in donor LysMcre/p53^{Loxp} mice for 28 days and then Ly6c⁺CD11c^{NEG}CD11b⁺Gr-1⁺ MDSCs sorted from spleen. Sorted cells were injected intravenously into WT recipients (CD45.1 congenic) bearing established tumors, and mice treated with immunogenic oxaliplatin. FACS plot shows p53 expression in transferred cells in treated tumors versus endogenous host mononuclear cells in the same tumor. Scatterplots show expression of key maturation markers in the endogenous host CD11⁺ dendritic cells versus the transferred LysMcre/p53^{Loxp} cells, both measured in the same tumor. *p<0.01 by ANOVA.

FIG. 6D illustrates B16F10 tumors grown in LysMcre/p53^{Loxp} mice vs WT controls and then treated with CTX/VO-OHpic. Each data-point is the mean of 10-14 tumors; * p<0.001 for treated LysMcre/p53^{Loxp} vs treated WT by ANOVA.

FIG. 6E illustrates WT (p53-sufficient) Ly6c⁺ precursor cells sorted from spleens of tumor-bearing hosts. EL4 tumors were used because they elicit large numbers of MDSCs. Sorted Ly6c+CD11c^{NEG}CD11b⁺Gr-1⁺ cells were transferred into tumor-bearing LysMcre/p53^{Loxp} mice, then treated as shown.

FIG. 6F illustrates LysMcre/p53^{Loxp} mice or WT controls received B16F10 tumors and then treated with combination immunotherapy comprising adoptive transfer of resting pmel-1 cells (CFSE-labeled, Thy1.1⁺), hgp100 vaccine, and dual checkpoint-blockade (monoclonal antibodies against CTLA-4 and PD-1/L pathway). Each data-point is the mean±SD of 8-10 tumors. *p<0.001. FACS plots are representative of 3 experiments.

FIG. 7 illustrates increased p53 levels in dendritic cells and myeloid cells in tumors following treatment with nutlin-3a. Histograms show p53 expression in gated CD11c⁺ cells in tumor, Ly6c⁺CD11c^{NEG} immature myeloid cells, and Gr-1HI cells. The remaining cells (negative for CD11c/Ly6c/Gr 1) expressed lower levels of p53, and these cells showed little effect of nutlin. Quantitation for three independent experiments is shown in the scatterplot.

FIGS. 8A-8D illustrate systemic activation of p53 using nutlin-3a promotes differentiation of Ly6c⁺/CD103⁺ dendritic cells and enhances response to immunotherapy.

FIG. 8A illustrates established B16F10 tumors treated with dual checkpoint blockade with or without addition of nutlin-3a (10 mg/kg/day i.p.). n=6 tumors/data point pooled from 3 experiments. *p<0.001 on day 22 by t-test. FACS plots show dendritic cells in tumors on day 22; representative of 4 experiments.

FIG. 8B illustrates established B16F10 tumors treated with CTX/VO-OHpic, with or without nutlin-3a, and followed for re-growth. Mean of 8-10 tumors per data point, pooled from 3 experiments, bars show SD; *p<0.001 by t-test.

FIG. 8C illustrates mice with EL4-OVA tumors (E.G7) treated with one dose of oxaliplatin (5 mg/kg) with or without nutlin-3a. Each data point is the mean±SD of 10-14 tumors pooled from 4 experiments. FACS plots show representative staining of CD11c⁺ dendritic cells on day 28. Scatter-plots show phenotype of the CD11c⁺CD103⁺ population, pooled from 5-9 experiments each. * p<0.01 by ANOVA.

FIG. 8D illustrates LysMcre/p53^{Loxp} P hosts or VVT controls were implanted with EL4-OVA tumors and treated with oxaliplatin chemotherapy with or without nutlin-3a. Mean of 6-8 tumors per data point pooled from 4 experiments, bars show SD; *=LysMcre/p53^{Loxp} groups not significantly different from each other. FACS analysis confirms loss of Ly6c+/CD103+ dendritic cells in the absence of p53; representative of 3 experiments.

FIG. 9 schematically illustrates self-amplifying cascade of anti-tumor immunity.

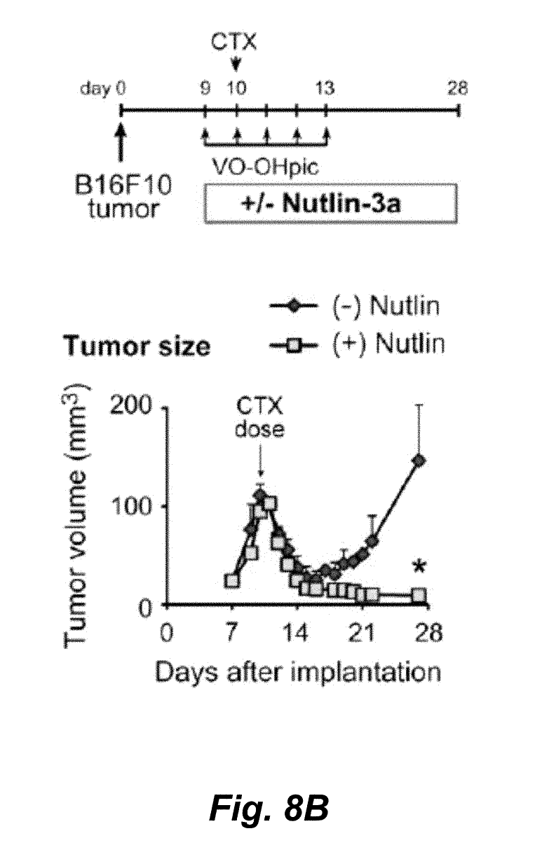

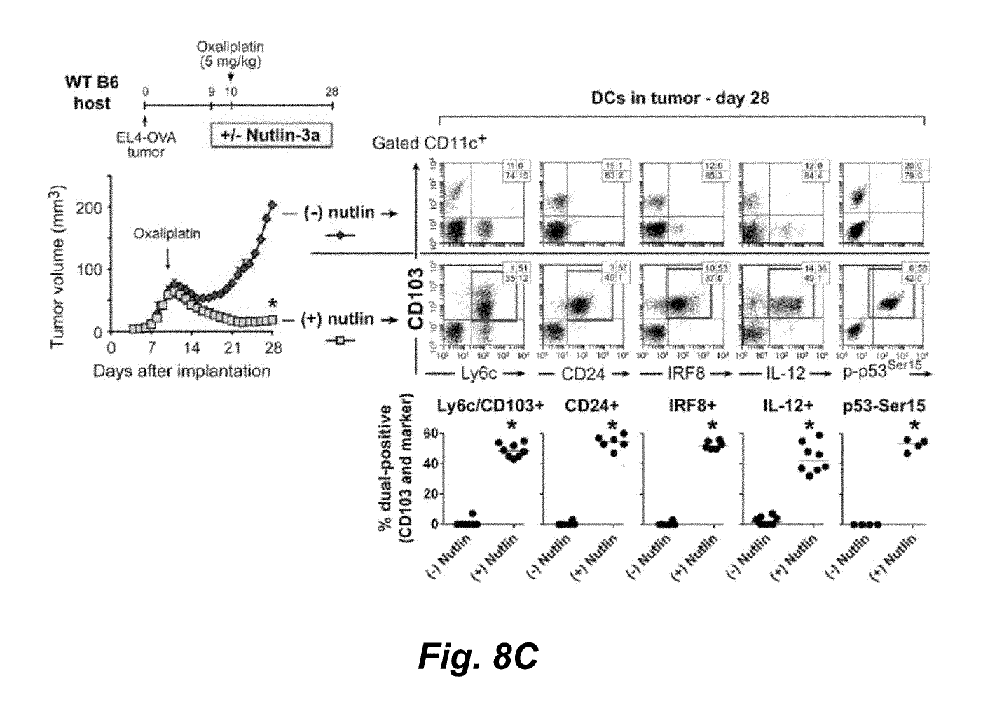

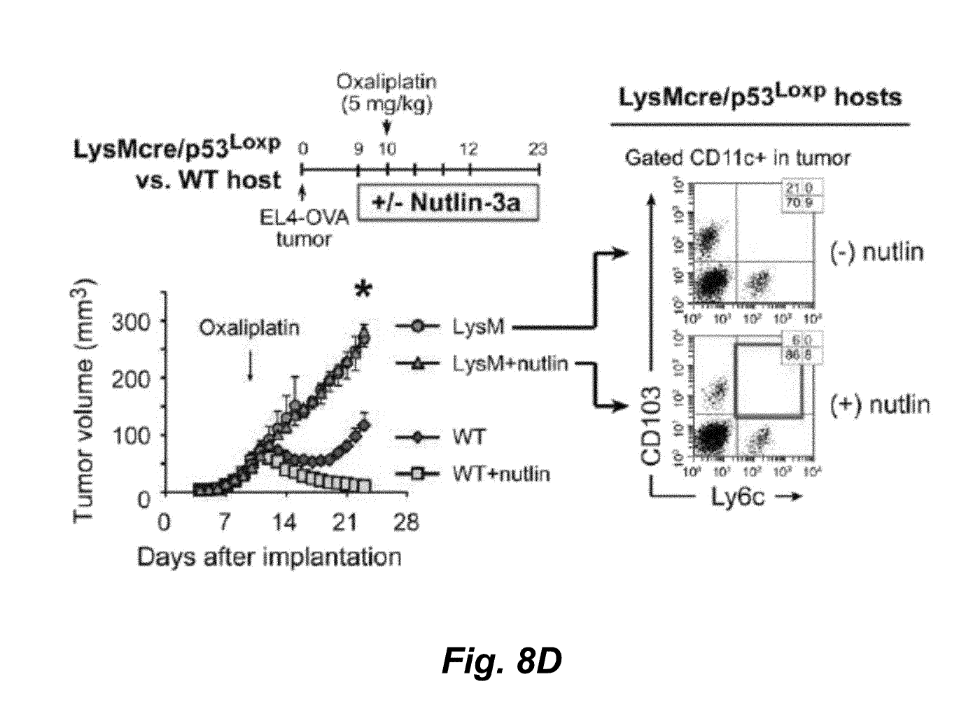

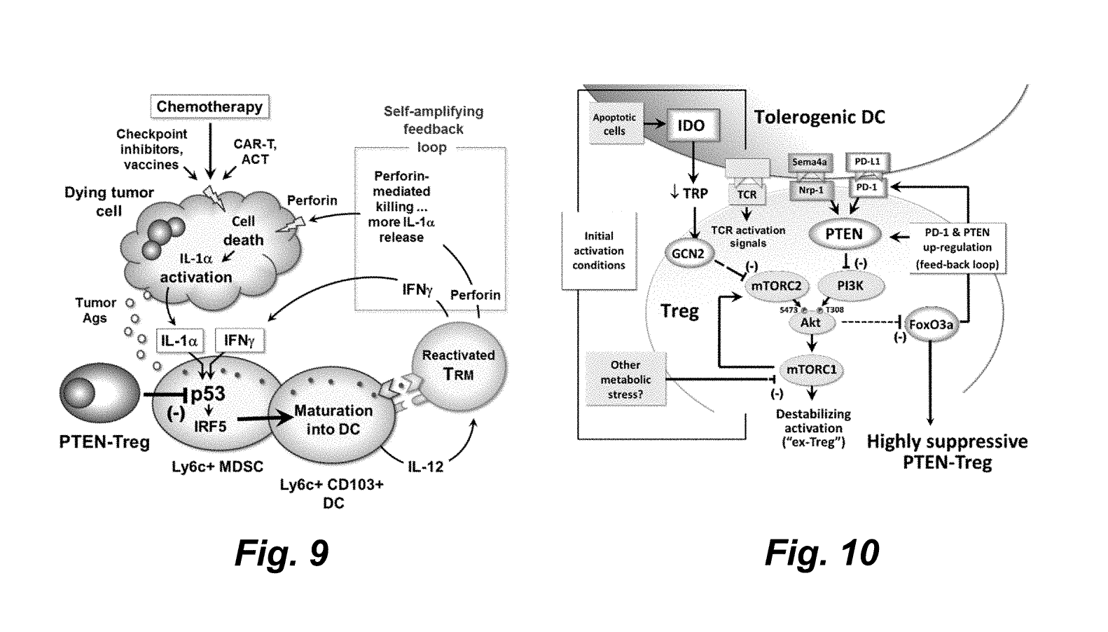

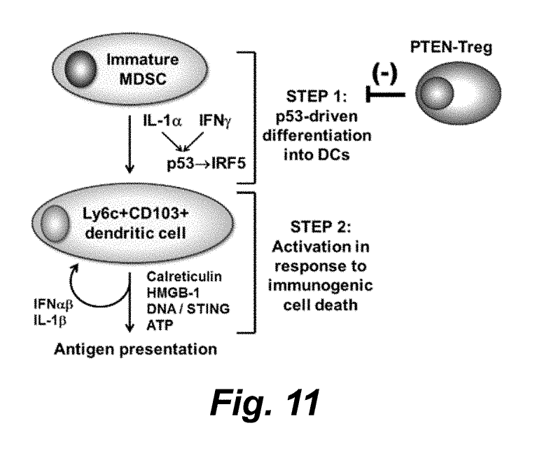

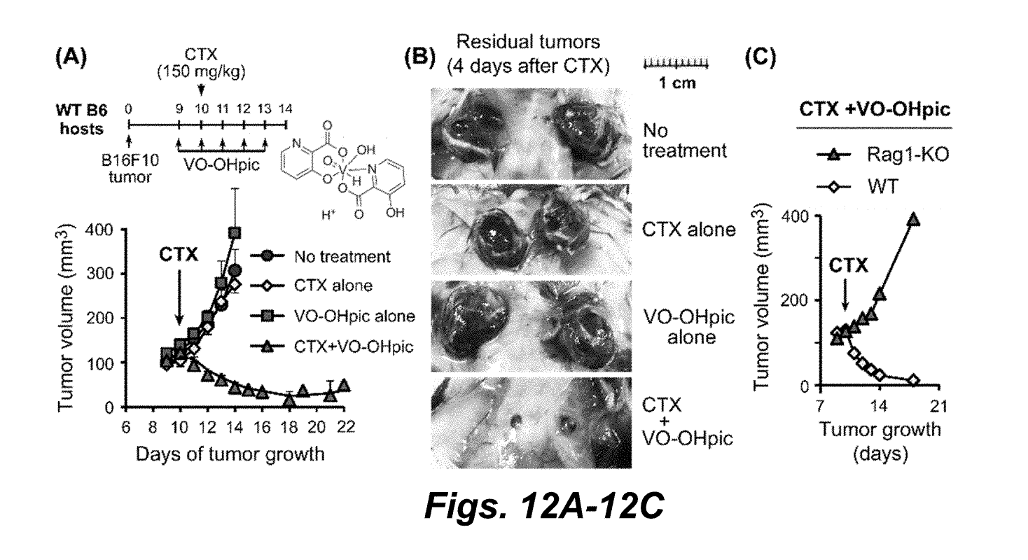

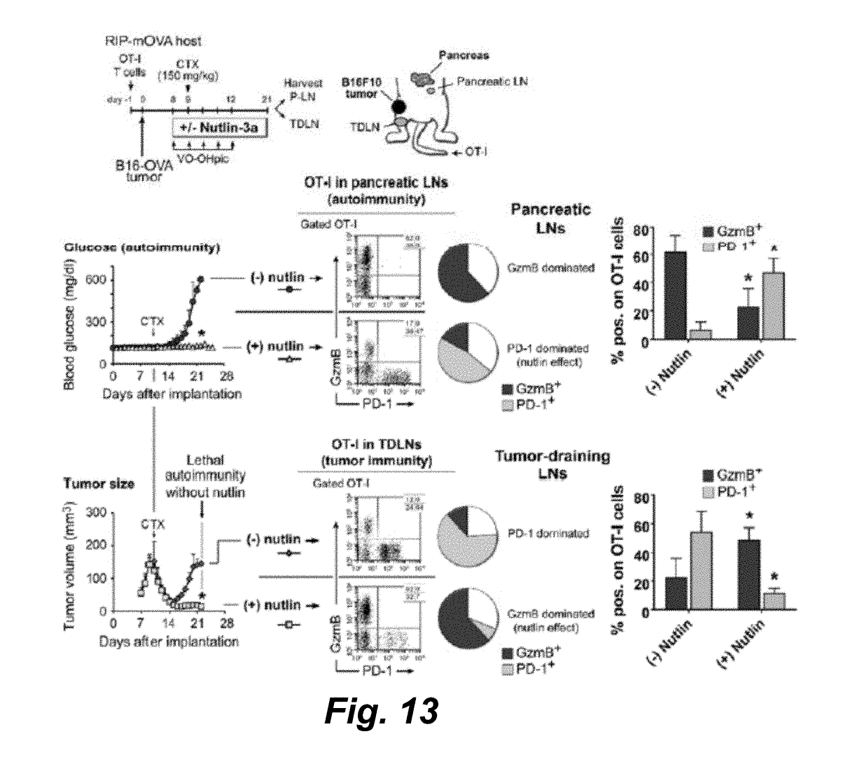

FIG. 10 schematically illustrates that PTEN lies downstream of IDO and GCN2 and maintains the long-term suppressive activity of IDO-activated Tregs.

FIG. 11 schematically illustrates that PTEN-Tregs act very early (immediately after the cells die) to suppress the initial maturation step of the critical cross-presenting Ly6c⁺CD103⁺ dendritic cells.

FIG. 12A illustrates that treatment with a PTEN-inhibitor drug (VO-OHpic) could overcome pre-existing suppression.

FIG. 12B illustrates that when the PTEN-inhibitor was combined with chemotherapy there was dramatic synergy.

FIG. 12C illustrates that all anti-tumor effect of CTX+VO-OHpic was lost in Rag1-deficient mice.

FIG. 13 illustrates RIP-mOVA mice expressing membrane-bound OVA as a self-antigen in the pancreas received pre-transfer of OVA-specific OT-I^{Thy1.1} T cells, then were implanted with B16-OVA tumors. Beginning on day +8, tumors were treated with VO-OHpic/CTX, with or without daily nutlin-3a injection (10 mg/kg/day). Blood glucose (upper graph) and tumor size (lower graph) are shown. Mean of 12 tumors per data point from 4 experiments, bars show SD; *p<0.001 by ANOVA. For FACS plots, the transferred OT-I cells were analyzed both in the draining LNs of pancreas (upper FACS plots) and in TDLNs (lower plots). Pie charts summarize the phenotype in the experiment shown; bar graphs show mean±SD of 5-9 pooled experiments for each marker.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “ includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### Abbreviations

DC, dendritic cell; MDSC, myeloid derived suppressor cell; LN, lymph node; TDLN, tumor-draining lymph node; TCR, T cell antigen receptor; APC, antigen-presenting cells; MDM2, Mouse Double Mutant 2; FACS, Fluorescence activated cell sorting; VVT, wild-type; CTX, cyclophosphamide; siRNA, short interfering RNA; i.v. intravenous(ly); ROS, reactive oxygen species; DCFDA, 2′,7′-dichlorofluorescein diacetate D1MT, 1-methyl-D-tryptophan; IFN, interferon; IDO, indoleamine 2,3-dioxygenase; CDP, committed dendritic cell progenitor cell.

### Definitions

The articles “a” and “an” as used herein refer to one or to more than one (i.e., to at least one) of the grammatical object of the article. By way of example, “an element” means one element or more than one element.

The term “including” as used herein refers to, and is used interchangeably with, the phrase “including but not limited” to.

The term “or” as used herein refers to, and is used interchangeably with, the term “and/or,” unless context clearly indicates otherwise.

The term “such as” as used herein refers to, and is used interchangeably, with the phrase “such as but not limited to”.

The terms “administration” and “administrating” as used herein refer to introducing a composition (e.g., a vaccine, adjuvant, immunogenic composition, small molecule therapeutic agent, and the like) of the present disclosure into a subject human or animal patient. A preferred route of administration of the therapeutic compositions of the disclosure is intravenous. However, any route of administration, such as oral, topical, subcutaneous, peritoneal, intra-arterial, inhalation, vaginal, rectal, nasal, introduction into the cerebrospinal fluid, or instillation into body compartments, can be used. Compositions and doses of the disclosure are administered in accordance with good medical practices taking into account the subject's clinical condition, the site and method of administration, dosage, patient age, sex, body weight, and other factors known to physicians.

The term “cancer”, as used herein, shall be given its ordinary meaning, as a general term for diseases in which abnormal cells divide without control. In particular, cancer may refer to angiogenesis related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have within it different populations of cells having differing processes that have gone awry. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wlms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

The terms “co-administration” or “co-administering” as used herein refer to the administration of a first therapeutic agent that provides an immune response, an elevated immune response (elevated with respect to an immune response in the absence of the agent), or a cytotoxic therapeutic agent, and an inhibitor of an MDM2-related protein as two separate formulations or as one single formulation.

The co-administration can be simultaneous or sequential in either order, wherein preferably there is a time period while both (or all) active agents simultaneously exert their biological activities. The first therapeutic agent that provides, for example, an immune response and the MDM2-related protein inhibitor can be co-administered either simultaneously or sequentially (e.g. intravenously) through a continuous infusion (one for the first therapeutic agent and eventually one for the MDM2-related protein inhibitor; or e.g. the first therapeutic agent is administered intravenously through a continuous infusion and said MDM2 inhibitor is administered orally). When both therapeutic agents are co-administered sequentially the dose is administered either on the same day in two separate administrations, or one of the agents is administered on day 1 and the second is co-administered on day 2 to day 7, preferably on day 2 to 4. Thus in one embodiment, the term “sequentially” means within 7 days after the dose of the first therapeutic agent or the MDM2-related protein inhibitor), preferably within 4 days after the dose of the first component; and the term “simultaneously” means at the same time. The terms “co-administration” with respect to the maintenance doses of the first therapeutic agent and the MDM2-related protein inhibitor mean that the maintenance doses can be co-administered simultaneously, if the treatment cycle is appropriate for both drugs, e.g. every week. Alternatively, the MDM2-related protein inhibitor may be administered such as every first to third day and the first therapeutic agent is administered every week. Alternatively, the maintenance doses may be co-administered sequentially, either within one or within several days.

The term “composition” as used herein refers to a product comprising the specified ingredients in the specified amounts as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other therapeutic agents, a pharmaceutical composition containing such other drugs in addition to the compound of the present disclosure is contemplated. Accordingly, the pharmaceutical compositions of the present disclosure include those that also contain one or more other active ingredients in addition to a compound of the present disclosure. The weight ratio of a first therapeutic agent to the second active ingredient may be varied and will depend upon the effective dose of each ingredient. Generally, an effective dose of each will be used. Thus, for example, but not intended to be limiting, when a first therapeutic agent such as an immunotherapeutic agent of the present disclosure is combined with another therapeutic agent, the weight ratio of the compound of the present disclosure to the other agent will generally range from about 1000:1 to about 1:1000, preferably about 200:1 to about 1:200. Combinations of a therapeutic agent of the present disclosure and other active ingredients will generally also be within the aforementioned range, but in each case, an effective dose of each active ingredient should be used. In such combinations the therapeutic agent of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion, tablet, pill, capsule, sustained release formulation, or powder. The compositions can be formulated as a suppository with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g. encapsulation in liposomes, microparticles, microcapsules, and the like. A composition of the disclosure may be sterilized by, for example, filtration through a bacteria-retaining filter, addition of sterilizing agents to the composition, irradiation of the composition, or heating the composition. Alternatively, the therapeutic agent or agents of the present disclosure may be provided as sterile solid preparations, e.g. lyophilized powder, which are readily dissolved in sterile solvent immediately prior to use.

A compound of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration and consistent with conventional pharmaceutical practices.

The term “formulation” as used herein refers to a composition that may be a stock solution of the components, or a composition that preferably includes a dilutant such as water or other pharmaceutically acceptable carrier and which may be available for distribution including to a patient or physician.

The term “homolog” as used herein includes, but is not limited to, amino acid sequences containing one or more amino acid substitutions, insertions, and/or deletions from a reference sequence and has a similar biological activity or function as the reference sequence. Amino acid substitutions may be of a conserved or non-conserved nature. Conserved amino acid substitutions involve replacing one or more amino acids of the proteins of the invention with amino acids of similar charge, size, and/or hydrophobicity characteristics. When only conserved substitutions are made, the resulting analog should be functionally equivalent. Non-conserved substitutions involve replacing one or more amino acids of the amino acid sequence with one or more amino acids which possess dissimilar charge, size, and/or hydrophobicity characteristics. Amino acid insertions may consist of single amino acid residues or sequential amino acids ranging from 2 to 15 amino acids in length. Deletions may consist of the removal of one or more amino acids or discrete portions from the amino acid sequence. The deleted amino acids may or may not be contiguous.

The term “immunotherapeutic agent” as used herein refers to any agent, compound, or biologic which is capable of modulating the host's immune system. For example, an immunotherapeutic agent is capable of causing a stimulation of the immune system against a tumor cell. The term “immunotherapeutic agent” can refer to an “immunostimulatory agent” which is an agent that modulates an immune response to an antigen but is not the antigen or derived from the antigen. The term “immunotherapeutic agent” as used herein can further refer to a cytotoxic chemotherapeutic agent that can induce tumor cell apoptosis and tumor cell death, resulting in invasion of the tumor by T-cells and inducing an immune response to the dead and dying tumor cells. Cytotoxic agents include, but are not limited to, the anthracyclines such as doxorubicin, idarubicin, and mitoxantrone that are apoptosis inducers. Anthracyclines are capable of eliciting immunogenic apoptosis eliciting immunogenic cell death.

The term “cytotoxic” as used herein refers to a moiety, compound, drug or agent that inhibits or prevents the function of cells and/or causes destruction of cells.

The term “IDO inhibitor” as used herein refers to an agent capable of inhibiting the activity of indoleamine 2,3-dioxygenase (IDO) and thereby reversing IDO-mediated immunosuppression. The IDO inhibitor may inhibit IDO1 and/or IDO2 (INDOL1). An IDO inhibitor may be a reversible or irreversible IDO inhibitor. “A reversible IDO inhibitor” is a compound that reversibly inhibits IDO enzyme activity either at the catalytic site or at a non-catalytic site, and “an irreversible IDO inhibitor” is a compound that irreversibly destroys IDO enzyme activity by forming a covalent bond with the enzyme.

IDO inhibitors may include, without limitation, previously established (known) IDO inhibitors, including, but not limited to: 1-methyl-DL-tryptophan (1MT) (as disclosed in U.S. Pat. No. 8,232,313), β-(3-benzofuranyl)-DL-alanine, β- (3-benzo(b)thienyl)-DL-alanine, 6-nitro-L-tryptophan, indole 3-carbinol, 3,3′-diindolylmethane, epigallocatechin gallate, 5-Br-4-CI-indoxyl 1,3-diacetate, 9-vinylcarbazole, acemetacin, 5-bromo-DL-tryptophan, 5-bromoindoxyl diacetate, and the IDO inhibitors provided in, for example, PCT/US04/05155, PCT/US04/05154, PCT/US06/42137, and U.S. patent application Ser. No. 11/589,024.

The term “myeloid derived suppressor cell (MDSC)” as used herein refers to a cell with an immunosuppressive function that is of hematopoietic lineage.

The term “MDM2-related protein” as used herein refers to proteins that have at least 25% sequence homology with MDM2 and interact with, and inhibit, p53 or p53-related proteins. Examples of MDM2-related proteins include, but are not limited to, MDMX and HDM2. MDM2 (synonyms: E3 ubiquitin-protein ligase Mdm2 p53 binding protein) is a p53-associated protein (Oliner et al., (1992) Nature 358: 80-83; Momand et al., (1992) Cell 69: 1237-1245; Chen et al., (1993) Mol. Cell. Biol. 13: 4107-4114; Bueso-Ramos et al., (1993) Blood 82: 2617-2623). It is a nuclear phosphoprotein that binds and inhibits transactivation by tumor protein p53 as part of an autoregulatory negative feedback loop. Overexpression of this gene or the protein can result in excessive inactivation of tumor protein p53, thereby diminishing its tumor suppressor function. This protein has E3 ubiquitin ligase activity, which targets tumor protein p53 for proteasomal degradation. This protein also affects the cell cycle, apoptosis, and tumorigenesis through interactions with other proteins, including retinoblastoma 1 and ribosomal protein L5.

The term “MDM2 inhibitor” as used herein refers to (therapeutic) agents that inhibit the MDM2-p53 interaction. Besides peptides and antibodies, several classes of small-molecule inhibitors with distinct chemical structures have now been reported including, but not limited to, a benzodiazepinedione, a sulphonamide; a chromenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, and stictic acid. (Shangary et al., (2008) Ann. Rev. Pharmacol. Toxicol. 49: 223-241). These are derivatives of cis-imidazoline (see e.g. Vassilev et al., (2004) Science 303: 844-848), spiro-oxindole (Ding et al., (2005) J. Am. Chem. Soc. 127: 10130-10131; Shangary et al., (2008) Proc. Natl. Acad. Sci. USA 105: 3933-3938; Ding et al., (2006) J. Med. Chem. 49: 3432-3435; Shangary et al., (2008) Mol. Cancer Ther. 7: 1533-1542), benzodiazepinedione (Grasberger et al., (2005) J. Med. Chem. 48: 909-912; Parks et al., (2005) Bioorg. Med. Chem. Lett. 15: 765-770; Koblish et al., (2006) Mol. Cancer Ther. 5: 160-169), terphenyl (Yin et al., (2005) Angew. Chem. Int. Ed. Engl. 44: 2704-2707; Chen et al., (2005) Mol. Cancer Ther. 4: 1019-1025), quilinol (Lu, Y., (2006) J. Med. Chem. 49: 3759-3762), chalcone (Stoll et al, (2001) Biochemistry 40: 336-44) and sulfonamide (Galatin et al., (2004) J. Med. Chem. 47: 4163-4165). Examples of MDM2 inhibitors and their mechanism of action and effect on p53 levels are also discussed in Hoe et al., (2014) Nature Revs. 13: 217-236, incorporated herein by reference in its entirety.

The term “modulate” as used herein can refer to inducing, enhancing, suppressing, directing, or redirecting an immune response.

The term “nutlin” as used herein refers to cis-imidazoline analogs that inhibit the interaction between MDM2 and tumor suppressor p53, and which were discovered by screening a chemical library by Vassilev et al., (2004) Science 303: 844-848. Nutlin-1, Nutlin-2 and Nutlin-3 were all identified in the same screen. However, Nutlin-3, (((±)-4-[4,5-Bis(4-chlorophenyl)-2-(2-isopropoxy-4-methoxy-phenyl)-4,5-dihydro-imidazole-1-carbonyl]-piperazin-2-one) is the compound most commonly used in anti-cancer studies. Inhibiting the interaction between MDM2 and p53 stabilizes p53 and is thought to selectively induce a growth-inhibiting state called senescence in cancer cells. These compounds are, therefore, thought to work best on tumors that contain normal or “wild-type” p53. Nutlin-3 has been shown to affect the production of p53 within minutes. The term “nutlin” as used herein further refers to enantiomers and stereoisomers. The more potent of the two enantiomers, (−)-Nutlin-3 (Nutlin-3A) ((−)-4-(4,5-Bis(4-chlorophenyl)-2-(2-isopropoxy-4-methoxyphenyl)-4,5-dihydro-1H-imidazole-1-carbonyl)piperazin-2-one can now be synthesized in a highly enantioselective fashion and is arbitrarily referred to as enantiomer because it appears as the first peak from chiral purification of racemic nutlin-3 although its absolute stereocenter assignment is not known. The term “nutlin” may further refer to “second-generation” nutlin” derivatives such as, but not limited to, RG7388 (ChemieTek, Indianapolis, IN) (described by Ding et al. (2013) J Med Chem. 56: 5979-5983 and incorporated herein by reference in its entirety) and to derivatives described in, for example, US Patent Applications 20150211073 and 20170008904.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “pharmaceutically acceptable carrier, excipient, or vehicle” as used herein refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “p53” as used herein refers to a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor that is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation. The ratio of MDM2 to p53 (E2F) is dysregulated in many cancers. Frequently occurring molecular defects in the p16lNK4/p19ARF locus, for instance, have been shown to affect MDM2 protein degradation. Inhibition of MDM2-p53 interaction in tumor cells with wild-type p53 should lead to accumulation of p53, cell cycle arrest, and/or apoptosis. MDM2 antagonists, therefore, provide an approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as does p53 and activates E2F dependent transcription of cyclin A, suggesting that MDM2 antagonists might have effects in p53 mutant cells.

The term “small molecule” as used herein refers to compounds that are typically organic, non-peptide molecules, having a molecular weight less than 10,000 Da, preferably less than 5,000 Da, more preferably less than 1,000 Da, and most preferably less than 500 Da. This class of modulators includes chemically synthesized molecules, for instance, compounds from combinatorial chemical libraries. Synthetic compounds may be rationally designed or identified utilizing screening methods. Alternative appropriate modulators of this class are natural products, particularly secondary metabolites from organisms such as plants or fungi, which can also be identified by screening compound libraries for tumor-killing activity. Methods for generating and obtaining small molecules are well known in the art.

The terms “subject,” “patient”, or “individual” as used herein refer to an animal having an immune system, preferably a mammal (e.g., rodent, such as mouse). In particular, the term refers to humans. As used herein, the term “mammal” has its ordinary meaning, and specifically includes primates, and more specifically includes humans. Other mammals that may be treated for the presence of a tumor, or in which tumor cell growth may be inhibited, include, but are not limited to, canine, feline, rodent (racine, murine, lupine, etc.), equine, bovine, ovine, caprine, and porcine species. The term “patient in need thereof” as used herein refers to a subject, animal or human, diagnosed with a disorder or suspected of having a disorder.

The terms “pharmaceutical agent”, “therapeutic agent” and “drug” are used herein interchangeably. They refer to a substance, molecule, compound, agent, factor or composition effective in the treatment, inhibition, and/or detection of a disease, disorder, or clinical condition.

The term “therapeutic effect” as used herein refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The term “therapeutic effect” as used herein also refers to an effect of a composition of the disclosure, in particular a formulation or dosage form, or method disclosed herein. A therapeutic effect may be a sustained therapeutic effect that correlates with a continuous concentration of a compound of the disclosure over a dosing period, in particular a sustained dosing period. A therapeutic effect may be a statistically significant effect in terms of statistical analysis of an effect of a compound of the disclosure versus the effects without the compound.

The term “therapeutically effective amount” as used herein refers to that amount of the therapeutic agent (including the compounds, pharmaceutical compositions, and compositions of matter provided herein) sufficient to result in amelioration of one or more symptoms of a disorder, prevent advancement of a disorder, or cause regression of the disorder. For example, with respect to the treatment of cancer, in one embodiment, a therapeutically effective amount can refer to the amount of a therapeutic agent that decreases the rate of tumor growth, decreases tumor mass, decreases the number of metastases, increases time to tumor progression, increase tumor cell apoptosis, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

When a combination of active ingredients is administered, the effective amount of the combination may or may not include amounts of each ingredient that would have been effective if administered individually. The dosage of the therapeutic formulation will vary depending upon the nature of the disease or condition, the patient's medical history, the frequency of administration, the manner of administration, the clearance of the agent from the host, and the like. The initial dose may be larger, followed by smaller maintenance doses. The dose may be administered, e.g., weekly, biweekly, daily, semi-weekly, etc., to maintain an effective dosage level.

Therapeutically effective dosages can be determined stepwise by combinations of approaches such as (i) characterization of effective doses of the composition or compound in in vitro cell culture assays using tumor cell growth and/or survival as a readout followed by (ii) characterization in animal studies using tumor growth inhibition and/or animal survival as a readout, followed by (iii) characterization in human trials using enhanced tumor growth inhibition and/or enhanced cancer survival rates as a readout.

The terms “treating” or “treatment” as used herein refer to (1) preventing or delaying the appearance of clinical symptoms of a state, disorder or condition developing in a human or other mammal that may be afflicted with or predisposed to the state, disorder or condition but does not yet experience or display clinical or subclinical symptoms of the state, disorder or condition, (2) inhibiting a state, disorder or condition, i.e., arresting, reducing or delaying the development of the disease or a relapse thereof (in case of maintenance treatment) or at least one clinical or subclinical symptom thereof, or (3) relieving the disease, i.e., causing regression of the state, disorder or condition or at least one of its clinical or subclinical symptoms.

The term “unit dosage form” as used herein refers to physically discrete units suitable as unitary dosages for human patients and other mammals with each unit containing a predetermined quantity of active material calculated to produce the desired therapeutic effect in association with suitable pharmaceutical carriers or excipients. The compositions according to the present disclosure may be formulated in a unit dosage form. A single daily unit dose also may be divided into 2 or 3 unit doses that are taken at different times throughout the day, or as a controlled release form, so as to reduce adverse side-effects as much as possible.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

### Description

The present disclosure encompasses embodiments of a method for enhancing a therapeutic treatment of a cancer by administering to a patient in need a second therapeutic agent that elevates protein p53. While most advantageous for use with immunotherapeutic agents, the method of the disclosure may also be usefully applied to the use of chemotherapeutic (cytotoxic agents).

In particular, the disclosure encompasses embodiments of a method of enhancing therapeutic agent effects by also administering to the patient in need a therapeutic agent that elevates the level of cell-intrinsic p53 in myeloid cells infiltrating the targeted tumor. It has now been found that increase in the amount of p53 in myeloid cells infiltrating the tumor initiates the formation of a specific population of dendritic cells that potentiates the induced antitumor immune reaction while suppressing undesirable autoimmune activity resulting from the immunotherapy.

CD103⁺ dendritic cells are critical for cross-presentation of tumor antigens to T cells. It has now been shown, for example, that during immunotherapy large numbers of CD103⁺ dendritic cells can arise in murine tumors via direct differentiation of Ly6c⁺ myeloid precursor cells. Ly6c⁺/CD103⁺ dendritic cells can derive from bone-marrow monocytic progenitors (cMoPs) or from peripheral cells present within the myeloid-derived suppressor cell (MDSC) population. Maturation was controlled by inflammation-induced activation of the transcription factor p53 in the immature cells, which then drove up-regulation of Batf3 and acquisition of the CD103⁺ phenotype. Mice with a targeted deletion of p53 in myeloid cells selectively lost the Ly6c⁺/CD103⁺ dendritic cell population and became unable to respond to multiple forms of immunotherapy and immunogenic chemotherapy. Conversely, increasing p53 levels using a p53-agonist drug caused a sustained increase in Ly6c+/CD103⁺ dendritic cells in tumors, which markedly enhanced multiple forms of immunotherapy. Thus, p53-driven differentiation of monocyte-lineage CD103⁺ dendritic cells is an advantageous and previously unrecognized target for immunotherapy.

The present disclosure describes the novel identification of a population of CD103-expressing, Batf3-dependent dendritic cells in tumors that can arise from monocyte-lineage cells. These “Ly6c⁺/CD103⁺” dendritic cells can differentiate from bone-marrow cMoP progenitors, or they can also arise by rapid, direct maturation of Ly6c⁺ myeloid cells in the periphery. The importance of this latter pathway is that these peripheral precursor cells are already pre-positioned in tumors and TDLNs and can differentiate quickly (in less than 24 h) in response to the transient wave of tumor antigens released by chemotherapy or immunotherapy.

This previously unrecognized population of dendritic cells plays an important role in initiating anti-tumor immune responses. Using targeted deletion in LysMcre/p53^{Loxp} mice, it has been found that the Ly6c⁺/CD103⁺ population is critical for anti-tumor response to multiple models of immunotherapy and immunogenic chemotherapy. The anti-tumor activity in

LysMcre/p53^{Loxp} mice was restored by transfer of isolated wild-type splenic Ly6c⁺ precursor cells, so the effect of the knockout appeared on-target and specific for this key dendritic cell population. The importance of these cells was further reinforced by the fact that adoptive transfer of monocyte-lineage cMoP cells (which give rise to Ly6c⁺/CD103⁺ dendritic cells) was even more effective at restoring tumor immunity in Batf3-deficeint mice than was transfer of classical dendritic-lineage (CDP) progenitors.

Myeloid-derived Ly6c⁺/CD103⁺ dendritic cells constitute a new cell type. They may be related to the recently-described “Tip-DCs” (Marigo et al., (2016) Cancer Cell 30: 377-390) and also, perhaps, to other myeloid antigen-presenting cells (Ma et al., (2013) Immunity 38: 729-741) that can be seen in inflamed tumors. The classical CD103⁺ dendritic lineage is well established in its own right, and it is likely that not all of the CD103⁺ dendritic cell in tumors are derived via this new monocytic pathway. Rather, it is now shown that the Ly6c⁺/CD103⁺ dendritic cells emerged only in response to treatment-induce inflammation. Accordingly, these cells may play a critical “first-responder” role following tumor-cell death and are key for initiating the cancer-immunity cycle, a self-amplifying loop of tumor-cell killing, antigen cross-presentation and endogenous T cell activation that is required for robust anti-tumor immunity (Chen & Mellman (2013) Immunity 39: 1-10).

The Ly6c⁺/CD103⁺ dendritic cells are thus critical antigen-presenting cells. However, it has been shown that their maturation is normally potently suppressed by the PTEN⁺ Tregs found in tumors. To overcome this, either conventional immunotherapy must create sufficient inflammation to physiologically destabilize these PTEN⁺ Tregs; or they must be pharmacologically destabilized by blocking the PTEN pathway.

Maturation of the Ly6c⁺/CD103⁺ population was controlled by the transcription factor p53. This identifies myeloid-lineage p53 as a previously unsuspected target for immunotherapy. Using nutlin-3a, a prototypical p53-agonist (MDM2-inhibitor) drug, it was shown that maturation of Ly6c⁺/CD103⁺ dendritic cells in tumors, and consequent anti-tumor immunity, can be significantly enhanced by systemic treatment with an MDM2-inhibitor. A number of MDM2-inhibitor drugs are already in clinical trials for non-immunologic indications (Khoo et al., (2014) Nature Revs. Drug Discov. 13: 217-236). One concern with repurposing oncology drugs is toxicity. However, targeting the normal physiologic level of p53 in host myeloid cells (which already express p53, and in which the pathway is highly inducible) is easier than trying to enforce re-expression of p53 in abnormal tumor cells. Thus, the high doses of MDM2-inhibitorand prolonged administration required for targeting tumor cells are not necessary to target normal p53 in host immune cells.

Whenever multiple agents are combined for cancer immunotherapy, a concern is life-threatening autoimmunity (June et al., (2017) Nat. Med. 23: 540-547). In this regard p53 represents a unique target because of its inherently dichotomous role in the immune system. In non-tumor models p53 has been shown to be immunosuppressive and tolerogenic (Munoz-Fontela et al., (2016a) Nat. Rev. Immunol. 16: 741-750), yet p53 can also play a pro-inflammatory role during infection or immune surveillance (Miciak and Bunz (2016) Biochim. Biophys. Acta 1865: 220-227). Targeting the p53 pathway had context-specific immune effects, promoting activation of T cells against a shared-self antigen in the context of the tumor, but inhibiting activation against the same antigen in the context of normal tissues. This selectivity for tumor over normal tissues makes p53 an attractive target for clinical immunotherapy.

The aggressive immune stimulation needed to break tolerance to tumors may also trigger lethal loss of self-tolerance in normal tissues. The present disclosure provides evidence (Example 25, FIG. 13) that the transcription factor p53 expressed by host immune cells plays two diametrically opposing roles during cancer immunotherapy. In normal tissues, p53 expressed in T cells inhibited immune activation and suppressed lethal off-target autoimmunity. In the tumor, however, p53 expression in myeloid-lineage cells mediated the cells' rapid differentiation into highly immunogenic dendritic cells, which then drove robust local T cell activation and tumor regression. Pharmacologic elevation of p53 using an MDM2 inhibitor drug caused potent and selective enhancement of immune activation within the tumor, while simultaneously suppressing lethal autoimmunity in normal tissues, even against the same shared tumor/self-antigen.

p53 has been extensively studied in malignant and transformed cells, but its role in normal cells is only beginning to be understood. In the immune system, the immunosuppressive and anti-inflammatory role of p53 is well-known (Watanabe et al., (2014) Immunity 40: 681-691; Kawashima et al., (2013) J. lmmunol. 191: 3614-3623; Yoon et al., (2015) Science 349: 1261669; He et al., (2015) Cell Rpts. 13: 888-897), but there are also emerging hints that p53 may be pro-inflammatory in certain settings (Slatter et al., (2016) Oncolmmunology 5: e1112941; Lowe et al., (2014) Cancer Res. 74: 2182-2192).

To test the immunogenic role of host-derived p53 in tumors, it was first necessary to bypass the highly suppressive tumor microenvironment that usually inhibits attempted immune activation. It has now been shown that many mammalian tumors critically rely on activated regulatory T cells (Tregs) expressing the lipid-phosphatase PTEN (PTEN-Tregs) so as to create their suppressive milieu (Sharma et al., (2015) Science Advances 1: e1500845).

Tumors implanted in mice with a Treg-specific deletion of PTEN (PTEN^{Treg}-KO mice) are spontaneously immunogenic and cannot create the usual suppressive tumor microenvironment. A notable feature of the inflamed tumors in these mice was the presence of large numbers of activated CD11c⁺ dendritic cells. These dendritic cells displayed an unusual phenotype, expressing both myeloid markers (Ly6c, CD11b, Gr1) and markers of conventional cross-presenting dendritic cells (CD103, IRF8, CD24 and Flt3) (Grajales-Reyes et al., (2015) Nat. Immunol. 16: 708-717; Broz et al., (2014) Cancer Cell 26: 638-652); they also uniformly expressed the pro-inflammatory cytokine IL-12 (Zitvogel et al., (2014) Cancer Cell 26: 591-593). This set of markers would not normally occur together (Broz et al., (2015) Cancer. Immunol. Res. 3: 313-319; Satpathy et al., (2012) Nat. Immunol. 13: 1145-1154), but it invariably occurred on the dendritic cells that co-expressed both Ly6c and CD103 in tumors. These newly identified cells are referred herein as “Ly6c⁺CD103⁺ dendritic cells”.

Preliminary characterization revealed that the Ly6c⁺CD103⁺ dendritic cells expressed a number of genes that were known targets of p53. Flow-cytometric analysis of p53 activation (phosphorylation at the Ser15 site) showed that the Ly6c+CD103⁺ subset were the only dendritic cells with constitutive phosphorylation of p53-Ser15. Functionally, treatment of PTEN^{Treg}-KO mice in vivo with the p53 inhibitor pifithrin (Komarov et al., (1999) Science 285: 1733-1737) selectively prevented the emergence of the Ly6c⁺CD103⁺ dendritic cell subset and allowed tumors to escape immune surveillance. Taken together, these data suggested that p53 in dendritic cells plays an immune-activating role and consequently that therapeutic means of elevating p53 levels could, by inducing the generation of the Ly6c⁺CD103⁺ dendritic cell subset, enhance immunotherapeutic activity directed to a target tumor.

To test this hypothesis, mice were used that had a myeloid-specific deletion of p53. These mice had normal PTEN expression, so to elicit anti-tumor immune activation, LysMcre/p53-KO mice were treated with the PTEN-inhibitor drug VO-OHpic. In WT control mice, treatment with VO-OHpic suppressed tumor growth and allowed emergence of the characteristic Ly6c⁺CD103⁺ dendritic cells. It has previously been shown that Tregs in tumors are critically dependent on the PTEN pathway, whereas effector T cells do not express PTEN and are indifferent to the inhibitor (Sharma et al., (2015) Science Advances 1: e1500845); thus, blocking PTEN allows immune activation in the tumor similar to PTEN^{Treg}-KO mice). However, in LysMcre/p53-KO mice, the tumors were not suppressed and the Ly6c⁺CD103⁺ dendritic cells were absent.

Consistent with this, LysMcre/p53-KO mice were also unable to respond to PTEN-based immunotherapy against large established tumors. It has previously been shown that immunotherapy with a PTEN-inhibitor drug is potently synergistic with conventional chemotherapy (Sharma et al., (2015) Science Advances 1: e1500845). However, when mice lacked p53 in myeloid cells, all anti-tumor effect was lost, and the Ly6c⁺CD103⁺ dendritic cell population failed to differentiate.

Finally, in vitro co-culture models showed that the activated Ly6c⁺CD103⁺ dendritic cells could differentiate directly from Ly6c+MDSCs sorted from tumors (Ly6c+CD11c^{NEG} cells) when they were exposed to signals from activated T cells. This differentiation step was inhibited by the p53-inhibitor pifithrin. Thus, taken together, these data suggested that p53 expression in myeloid-lineage cells played an important and previously unsuspected role in the immune response against tumors.

Based on this, it was investigated whether p53 might be a target for immunotherapy. In most cells the level of p53 is controlled by the ubiquitin-ligase MDM2, and blocking MDM2 elevates p53 expression. A number of MDM2-inhibitors (“p53 agonists”) are in clinical trials with the goal of increasing p53 in tumor cells (Khoo et al., (2014) Drug Discovery 13: 217-236). These drugs have never been considered for immunotherapy because current theory would predict that they should inhibit T cell activation and suppress inflammation (Watanabe et al., (2014) Immunity 40: 681-691; Kawashima et al., (2013) J. Immunol. 191: 3614-3623; Yoon et al., (2015) Science 349: 1261669; He et al., (2015) Cell Rpts. 13: 888-897). However, the results as presented in the present disclosure suggested that within the tumor itself increasing host p53 might promote maturation of immune-activating, pro-inflammatory dendritic cells.

To ask which of these two effects prevailed, mice with established tumors were treated with immunotherapy (VO-OHpic+CTX) with or without the addition of nutlin-3a, a prototypical MDM2-inhibitor (p53-agonist) drug (Vassilev et al., (2004) Science 303: 844-848). Both groups initially showed good response, as expected. However, in mice without nutlin the tumors all began to re-grow about 2 weeks after immunotherapy (consistent with a previous report (Sharma et al., (2015) Science Advances 1: e1500845)). In contrast, mice that received nutlin-3a maintained a prolonged anti-tumor response, which was accompanied by a sustained elevation of phospho-p53-Ser15 in dendritic cells, with a large population of the p53-dependent Ly6c⁺CD103⁺ dendritic cells in tumors. Without nutlin, the Ly6c⁺CD103⁺ dendritic cells had almost completely disappeared by day 21.

Thus, nutlin-3a can act as a potent immune-enhancing drug in this model by inducing and expanding a population of dendritic cells unique to the tumor, namely the Ly6c⁺CD103⁺ dendritic cells. However, this result is surprising because nutlin has been shown in other settings to suppress T cell activation and, therefore, inhibit immune responses (Watanabe et al., (2014) Immunity 40: 681-691; Allam et al., (2011) J. Am. Soc. Nephrol. 22: 2016-2027).

Accordingly and without wishing to be bound by any one hypothesis, it is likely that p53 can play two different immunologic roles, depending on the context (tumor versus normal tissues). To test the suppressive role of p53 outside of the tumor, a model was used that exhibits aggressive autoimmunity in which ovalbumin (OVA)-specific OT-I T cells are adoptively transferred into mice expressing ovalbumin as a self-antigen on pancreatic islet cells (RIP-mOVA mice) (Kurts et al., (1998) J. Exp. Med. 188: 415-420). Following T cell transfer, these mice progressively developed lethal autoimmune diabetes. However, if mice were treated with nutlin-3a diabetes formation was suppressed and the mice remained healthy. Nutlin treatment increased the level of p53 in OT-I cells in pancreatic LNs, while decreasing the expression of the T cell activation marker granzyme B.

Thus, p53 appeared to be immune-activating within the tumor by the generation of the novel subset of dendritic cells that promote the intratumoral immune response, yet outside the tumor p53 is immunosuppressive and thereby reducing the extent of therapeutic treatments inducing autoimmunity.

It was then determined which of these opposing effects would prevail when the same antigen was both a tumor-antigen and self-antigen. RIP-mOVA mice expressing OVA as a self-antigen were implanted with B16-OVA tumors expressing OVA as a tumor antigen (Falo. Jr. et al., (1995) Nat. Med. 1: 649-653). Prior to tumor implantation, all mice received adoptive transfer of congenically-marked OT-I cells. After tumors were established, mice were treated with immunotherapy (PTEN-inhibitor plus chemotherapy), with or without nutlin-3a. In the absence of nutlin, immunotherapy caused tumors to shrink as expected, but the tumors soon re-grew. Simultaneously, the mice also rapidly developed lethal autoimmune diabetes. In contrast, when nutlin-3a was added to the immunotherapy, the anti-tumor effect was enhanced and prolonged, yet mice were fully protected from diabetes (immune activation was inhibited in the pancreas).

Comparison of OT-I T cells in pancreatic LNs and tumor-draining LNs (TDLNs) confirmed that nutlin had completely different effects on OT-I responses depending on the location. In pancreatic LNs the effect of nutlin was to increase p53 in OT-I. In the TDLNs of the same animals, the effect of nutlin was paradoxically to cause complete loss of detectable p53 expression. This was consistent with the dichotomous effects of nutlin on tumor immunity versus autoimmunity, but it raised the question of how a single drug could have diametrically opposite effects on the same cells in two different locations in the same animal.

It was noted that a major difference between the tumor and normal tissues was that nutlin promoted the sustained differentiation of many Ly6c⁺CD103⁺ dendritic cells in tumors.

These Ly6c⁺CD103⁺ dendritic cells expressed IL-12 that is an important enhancing signal for T cell activation (Tugues et al., (2015) Cell Death Differ. 22: 237-246). It is possible that IL-12 production by these Ly6c⁺CD103⁺ dendritic cells in tumors might rescue local T cells from suppression by nutlin.

Resting T cells express inhibitory p53 (Sturm et al., (2002) J. Clin. Invest. 109: 1481-1492) that must be down-regulated in order for T cells to divide (Watanabe et al., (2014) Immunity 40: 681-691). Under normal circumstances the signal to downregulate p53 is provided by engagement of the T cell antigen receptor (TCR) that activates MDM2 and targets p53 for degradation (Watanabe et al., (2014) Immunity 40: 681-691). Consistent with this model, activation of OT-I T cells with cognate antigen resulted in prompt down-regulation of p53 in the dividing cells. (In this model, although total p53 expression is only a proxy for its actual subcellular localization and function, it is an informative indicator of down-regulation.) Inhibiting MDM2 using nutlin-3a prevented p53 degradation, caused a marked increase in total p53 and prevented T cell proliferation (lower dot-plot and right-hand diagram).

Regulation of p53 is complex, and it can be targeted by multiple pathways besides MDM2 (Chao C. C. (2015) Clin. Chim. Acta 438: 139-147). If IL-12 is rescuing T cells from nutlin, one possibility is that IL-12 might activate an alternative pathway of p53 degradation, not dependent on MDM2. To test this, purified T cells were activated (without any antigen-presenting cells or other cells) using anti-CD3/CD28 antibodies. T cells were activated in the presence or absence of nutlin with or without addition of recombinant IL-12. Nutlin-3a suppressed proliferation of T cells and suppression was fully reversed by adding IL-12. As a control for the on-target specificity of nutlin, p53-deficient T cells showed no inhibitory effect of nutlin, and no activating effect of IL-12. Nutlin blocked the down-regulation of p53 during activation (resulting in elevated p53 levels). However, IL-12 bypassed the effect of nutlin, and allowed p53 to be down-regulated.

To test whether the effect of Ly6c⁺CD103⁺ dendritic cells was due to IL-12, OT-I T cells were activated using 3 different types of antigen-presenting cells (APCs). Using dendritic cells from normal LNs (which do not produce IL-12), activation of T cells caused down-regulation of p53, as expected. Down-regulation of p53 was completely prevented by nutlin. In contrast, when CD11c+Ly6c+ dendritic cells were used as antigen-presenting cells (sorted from TDLNs of mice treated with VO-OHpic+CTX), nutlin lost all ability to prevent down-regulation of p53.

Ly6c⁺CD103⁺ dendritic cells produce high levels of IL-12 and neutralization of IL-12 in co-cultures using a blocking antibody against IL-12p40 fully restored the ability of nutlin to increase p53 and inhibit T cells. Consistent with this, when dendritic cells were sorted from TDLNs of IL-12-deficient mice (IL-12p40-KO), treated with the same immunotherapy regimen, these dendritic cells were unable to rescue T cells from nutlin. T cell proliferation tracked concordantly with p53 levels. In these experiments with dendritic cells from TDLNs, feeder populations were added to maintain functional activity. Feeder cells did not affect the results.

Finally, to test the hypothesis that IL-12 targeted p53 for proteolytic degradation via an alternated pathway, proteasomal function was inhibited using MG-132. T cells were activated using immobilized CD3/CD28 as above. Compared to resting T cells, CD3 activation caused down-regulation of p53, while the addition of graded amounts of nutlin-3a progressively restored, and then markedly elevated, p53 levels. CFSE proliferation tracked inversely with p53 levels. Next, in the presence of nutlin, adding graded amounts of IL-12 forced progressive down-regulation of p53 and restored proliferation. Finally, in the presence of both nutlin and IL-12, blocking proteasomal degradation with MG-132 progressively increased p53 and inhibited proliferation. While MG-132 affects many intracellular pathways besides p53, these findings support the basic hypothesis that IL-12 bypasses nutlin by targeting p53 for degradation via an alternative route.

Taken together, these findings resolved the paradox of how nutlin could suppress T cells outside of the tumor, yet promote activation of the same T cells inside the tumor. In normal tissues, the relevant target of nutlin was the p53 expressed in T cells, and nutlin was directly suppressive. In the tumor milieu, with its chronic inflammation and accumulation of myeloid precursors, the main target of nutlin was the p53 in Ly6c⁺CD103⁺ dendritic cells. These dendritic cells then protected intratumoral T cells via production of IL-12 such that nutlin had no immunosuppressive effect inside the tumor and was purely stimulatory.

Finally, it was investigated whether the immunologic effects of p53 extended beyond the VO-OHpic/CTX model to widely used models of immunogenic cell death (ICD) and checkpoint blockade.

Most of standard chemotherapies induce a non-immunogenic apoptosis (Zitvogel et al., (2004) Science 305: 197-200; Lake & van der Most (2006) Engl J Med 354: 2503-4). Thus, even after an initially efficient chemotherapy, patients do not develop an efficient antitumorous immune response and then are overcome by chemotherapy-resistant tumorous variants. To improve anticancer chemotherapy, induction of immunogenic cancer-cell death is advantageous in that the immune system can contribute through a “bystander effect” to eradicate chemotherapy-resistant cancer cells and cancer stem cells (Steinman & Mellman (2004) Science 305: 197-200; Lake & van der Most (2006) Engl. J. Med. 354: 2503-2504; Apetoh, et al. (2007) Cancer Genomics Proteomics 4: 65-70).

The efficiency of a chemotherapeutic treatment and the responsiveness of a tumor thereto is linked to the choice of drugs used and to the molecules involved in the chemotherapy. The main drugs used in anti-tumorous chemotherapy can be divided in four groups: cytotoxic agents, hormones, immune response modulators, and inhibitors of the tyrosine kinase activity. Cytotoxic agents including, but not limited to, cytotoxic antibiotics such as anthracyclines (doxorubicin, idarubicin, and mitoxantrone which are apoptosis inducers). Anthracyclines are capable of eliciting immunogenic apoptosis (Casares et al., (2005) Exp Med. 202, 1691-701) and thus eliciting immunogenic cell death.

A classical model of ICD was first tested in which EL4-OVA lymphoma tumors (clone E.G7) are treated with oxaliplatin chemotherapy (Ghiringhelli et al., (2009) Nat. Med. 15: 1170-1178). E.G7 bears the same model antigen as B16-OVA (described with the RIP-mOVA model, above), so it was possible to test the effects of nutlin plus oxaliplatin in the RIP-mOVA model. In the absence of nutlin, treatment with oxaliplatin resulted in transient regression of E.G7 tumors, but these soon regrew, and the mice also rapidly died from progressive autoimmune diabetes. The addition of nutlin-3a treatment, however, protected the mice from diabetes while significantly enhancing the anti-tumor activity of the immunotherapy.

To ensure that these effects were not an artifact of the RIP-mOVA system, the model was tested in normal wild-type 057B1/6 hosts. In normal hosts, nutlin-3a still markedly prolonged the anti-tumor effect of a single immunogenic dose of oxaliplatin. This was accompanied by sustained expression of the characteristic Ly6c⁺CD103⁺ dendritic cells in tumor, with enhanced IL-12 production.

To test whether the relevant target of nutlin in tumors was specifically the p53 expression in myeloid dendritic cells, the E.G7/oxaliplatin model in LysMcre/p53-KO hosts was used. If mice lacked the relevant p53 in tumor-associated myeloid cells, then LysMcre/p53-KO mice should lose all anti-tumor effect of nutlin. For these studies, mice received congenically-marked OT-I cells to monitor whether T cells were still rescued by IL-12 in tumors. LysMcre/p53-KO mice were unable to respond to immunogenic oxaliplatin chemotherapy (a model known to require immune activation for the effects the chemotherapy (Ghiringhelli et al., (2009) Nat. Med. 15: 1170-1178)).

It was found that LysMcre/p53-KO mice lost all anti-tumor effects of nutlin-3a. LysMcre/p53-KO hosts failed to maintain Ly6c⁺CD103⁺ dendritic cells in the tumor, and none of the dendritic cells expressed IL-12. Importantly, in the absence of p53 in myeloid cells, the OT-I cells in tumors were not protected from nutlin and p53 was not downregulated. Thus, the ability of nutlin to enhance anti-tumor immunity specifically required its on-target effect on p53 in myeloid cells.

It was further investigated whether nutlin-3a could enhance the efficacy of a clinically-relevant immunotherapy approach (checkpoint blockade) while suppressing collateral autoimmunity. For this, CTLA-4 blockade was combined with blockade of the PD-1/PD-ligand pathway. Clinically, this combination shows the greatest efficacy to date, but it can be associated with significant undesirable immune-mediated toxicity (Larkin et al., (2015) N. Engl. J. Med. 373: 23-34).

RIP-mOVA mice bearing B16-OVA tumors were treated with a cocktail of blocking antibodies against CTLA-4 plus PD-1, PD-L1 and PD-L2, with or without concurrent nutlin-3a.

As with the other immunotherapy models, addition of nutlin protected recipient subjects from autoimmune diabetes, while significantly enhancing and prolonging the anti-tumor effect of checkpoint immunotherapy. Nutlin maintained expression of Ly6c⁺CD103⁺ dendritic cells in the tumors, and the dendritic cells continued to produce extensive IL-12.

Thus, taken together, the findings of the present disclosure identify p53 as a previously unsuspected target for immunotherapy with a unique dual mechanism of action. Within the tumor milieu, activating p53 with nutlin-3a enhanced the response to multiple different forms of immunotherapy while protecting normal tissues in the same animals from off-target autoimmunity. T cells in the tumor may be selectively protected from the suppressive effects of nutlin because of the contrary immune-activating effects of the p53-driven IL-12 production by Ly6c⁺CD103⁺ dendritic cells. The selective effects of p53 arose as a consequence of the tumors spontaneously recruiting many immature myeloid cells (Ugel et al., (2015) J. Clin. Invest. 125: 3365-3376), which then give rise to the Ly6c⁺CD103⁺ dendritic cells in a p53-dependent fashion.

The unique dual action of p53 is advantageous. Current immunotherapy is already close to the limits of usefulness due to the toxicity from off-target autoimmunity and inflammation, and only the most favorable patients, with the most immunogenic tumors, currently respond (McGranahan et al., (2016) Science 351: 1463-4169). Thus there remains an unfilled need for a mechanism to actively suppress the therapy-limiting autoimmunity while further enhancing on-target immune activation in the tumor.

Although the attributes of the novel dendritic cells of the disclosure are unusual compared to other subsets of dendritic cells, featuring markers of both monocytic MDSCs and conventional CD103⁺ cross-presenting dendritic cells, they are very similar to CD103⁺ IL-12-producing dendritic cells described before in tumors. The subset of Ly6c⁺CD103⁺ dendritic cells represent an important differentiation pathway for myeloid cells in the immune system. Activated immature myeloid cells are naturally recruited to sites of inflammation, where they normally differentiate into pro-inflammatory antigen-presenting cells. Although tumors are chronically inflamed, and they actively recruit many immature myeloid cells, the abnormal tumor microenvironment forces these myeloid cells to remain immature and immunosuppressive (Gabrilovich et al., (2012) Nat. Rev. lmmunol. 12: 253-268. We hypothesize that p53-driven differentiation into immunogenic Ly6c+CD103⁺ dendritic cells is actually the natural, physiologic terminal maturation step of this important population of myeloid antigen-presenting cells.

Pharmaceutical Formulations and Routes of Administration: Embodiments of the present disclosure include a composition or pharmaceutical composition as identified herein and can be formulated with one or more pharmaceutically acceptable excipients, diluents, carriers and/or adjuvants. In addition, embodiments of the present disclosure include a composition or pharmaceutical composition formulated with one or more pharmaceutically acceptable auxiliary substances. In particular the composition or pharmaceutical composition can be formulated with one or more pharmaceutically acceptable excipients, diluents, carriers, and/or adjuvants to provide an embodiment of a composition of the present disclosure. A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A.H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The compositions or pharmaceutical compositions of the disclosure can be administered to the subject using any means capable of resulting in the desired effect. Thus, the composition or pharmaceutical composition can be incorporated into a variety of formulations for therapeutic administration. For example, the composition or pharmaceutical composition can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. The composition or pharmaceutical composition of the disclosure may be administered in the form of its pharmaceutically acceptable salts, or a subject active composition may be used alone or in appropriate association, as well as in combination, with other pharmaceutically active compounds.

For oral preparations, the composition or pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and/or flavoring agents.

Embodiments of the composition or pharmaceutical composition can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the composition or pharmaceutical composition can be utilized in aerosol formulation to be administered via inhalation. Embodiments of the composition or pharmaceutical composition can be formulated into pressurized acceptable propellants such as dichlorodifluoromethane, propane, nitrogen and the like.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the composition or pharmaceutical composition in a composition as a solution in sterile water, normal saline or another pharmaceutically acceptable carrier.

Embodiments of the composition or pharmaceutical composition can be formulated in an injectable composition in accordance with the disclosure. Typically, injectable compositions are prepared as liquid solutions or suspensions; solid forms suitable for solution in, or suspension in, liquid vehicles prior to injection may also be prepared.

The compositions of the disclosure may be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Pumps provide consistent, controlled release over time. The compositions of the disclosure may be in a liquid formulation in a drug-impermeable reservoir and delivered in a continuous fashion to the individual.

A drug delivery system may be an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, in the brain ventricles, trans-nasally, or any other route to access directly the brain parenchyma, tumor or other suitable site within a subject's body. Subcutaneous implantation sites may be used because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

Dosages: Embodiments of the composition or pharmaceutical composition can be administered to a subject in one or more doses. Those of skill will readily appreciate that dose levels can vary as a function of the specific composition or pharmaceutical composition administered, the severity of the symptoms and the susceptibility of the subject to side effects. Preferred dosages for a given compound are readily determinable by those of skill in the art by a variety of means.

Multiple doses of the compositions of the disclosure may be administered. The frequency of administration of the compositions of the disclosure may be varied depending on any of a variety of factors, e.g., severity of the symptoms, and the like. For example, compositions of the disclosure may be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day. As discussed above, compositions of the disclosure may be administered 1 to 4 times a day over a 1 to 10 day time period, a 1 to 20 day time period, a 1 to 30 day time period, a 1 to 60 day time period, a 1 to 90 day time period, a 1 to 120 day time period, a 1 to 365 day time, or greater than 365 day period.

The duration of administration of the compositions of the disclosure, e.g., the period of time over which the compositions of the disclosure may be administered, can vary, depending on any of a variety of factors, e.g., patient response, etc. For example, compositions of the disclosure, in combination or separately, may be administered over a period of time of about one day to one week, about one day to two weeks, about one day to four weeks, about one day to one month, about one day to two months, about one day to three months, about one day to four months, about one day to five months, about one day to six months, about one day to one year, or more. The amount of the compositions of the disclosure that may be effective in treating the condition or disease may be determined by standard clinical techniques. In addition, in vitro or in vivo assays can optionally be employed to help identify optimal dosage ranges. The precise dose to be employed can also depend on the route of administration, and can be decided according to the judgment of the practitioner and each patient's circumstances.

Accordingly, one aspect of the disclosure encompasses embodiments of a method for enhancing a therapeutic treatment of a cancer, said method comprising the steps of: (a) administering to a patient in need thereof a therapeutic dose of a first therapeutic agent for the treatment of a cancer in said patient; and (b) administering to the patient a therapeutic dose of a second therapeutic agent that elevates the level of protein p53 in said patient.

In some embodiments of this aspect of the disclosure, the first therapeutic agent can be an immunotherapeutic agent or a cytotoxic agent.

In embodiments of this aspect of the disclosure, the second therapeutic agent can generate in a tumor a population of dendritic cells expressing at least one of Batf3, IRF5, CD103, and XCR1.

In some embodiments of this aspect of the disclosure, the second therapeutic agent can suppress an autoimmune response to non-cancerous tissue in the patient generated by the immunotherapeutic agent.

In some embodiments of this aspect of the disclosure, the first therapeutic agent can be an immunotherapeutic agent and the second therapeutic agent can enhance the immunotherapeutic response directed against a tumor in the patient.

In some embodiments of this aspect of the disclosure, the method can further comprise administering to the patient a therapeutic dose of a PTEN phosphatase inhibitor.

In some embodiments of this aspect of the disclosure, the second therapeutic agent can be an inhibitor of a Mouse Double Minute 2 (MDM2) (E3 ubiquitin-protein ligase) MDM2-related protein homolog.

In some embodiments of this aspect of the disclosure, the MDM2-related protein inhibitor can be a nutlin, a benzodiazepinedione, a sulphonamide; a chromenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, or stictic acid.

In some embodiments of this aspect of the disclosure, the MDM2-related protein inhibitor can be nutlin-3a.

In some embodiments of this aspect of the disclosure, the first therapeutic agent can be an indoleamine 2,3-dioxygenase (IDO) inhibitor.

In some embodiments of this aspect of the disclosure, the indoleamine 2,3-dioxygenase (IDO) inhibitor can be 1-methyl-D-tryptophan (D1MT), 1-cyclohexyl-2-(5H-imidazo[5,1-a]isoindol-5-yl)ethanol (GDC919/NLG919), or (E)-4-Amino-N′-(3-chloro-4-fluorophenyl)-N-hydroxy-1,2,5-oxadiazole-3-carboximidamide (INCB024360).

In some embodiments of this aspect of the disclosure, the first therapeutic agent can be an anthracene cytotoxic agent selected from the group consisting of doxorubicin, idarubicin, and mitoxantrone.

In some embodiments of this aspect of the disclosure, the first and the econd therapeutic agents can be individually administered to the patient.

In some embodiments of this aspect of the disclosure, the first and the second therapeutic agents can be administered in a single formulation.

In some embodiments of this aspect of the disclosure, the first and the second therapeutic agents and the PTEN phosphatase inhibitor can be individually administered to the patient.

In some embodiments of this aspect of the disclosure, the first and the second therapeutic agents and the PTEN phosphatase inhibitor are administered in a single formulation.

Another aspect of the disclosure encompasses embodiments of a composition comprising a first therapeutic agent for the treatment of a cancer in a recipient patient and a second therapeutic agent that elevates the level of protein p53 in said patient.

In some embodiments of this aspect of the disclosure, the first therapeutic agent can be an immunotherapeutic agent or a cytotoxic agent.

In some embodiments of this aspect of the disclosure, the composition can further comprise a PTEN phosphatase inhibitor.

In some embodiments of this aspect of the disclosure, the composition can further comprise a pharmaceutically acceptable carrier.

In some embodiments of this aspect of the disclosure, the composition can be formulated for delivering to a patient in need thereof an amount of an immunotherapeutic agent effective in generating an immune response directed against a tumor in the recipient patient and an amount of the second therapeutic agent effective in enhancing the immunotherapeutic response directed against a tumor of the patient by generating a population of dendritic cells expressing at least one of Batf3, IRF5, CD103, and XCR1 in the tumor.

In some embodiments of this aspect of the disclosure, the second therapeutic agent can further suppress an autoimmune response to non-cancerous tissue in the patient generated by the immunotherapeutic agent.

In some embodiments of this aspect of the disclosure, the second therapeutic agent can be an inhibitor of a Mouse Double Minute 2 (MDM2) (E3 ubiquitin-protein ligase) MDM2-related protein homolog.

In some embodiments of this aspect of the disclosure, the MDM2-related protein inhibitor can be a nutlin, a benzodiazepinedione, a sulphonamide; a chrornenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, or stictic acid.

In some embodiments of this aspect of the disclosure, the MDM2-related protein inhibitor can be nutlin-3a.

In some embodiments of this aspect of the disclosure, the immunotherapeutic agent is an indoleamine 2,3-dioxygenase (IDO) inhibitor.

In some embodiments of this aspect of the disclosure, the indoleamine 2,3-dioxygenase (IDO) inhibitor can be 1-methyl-D-tryptophan (D1MT), 1-cyclohexyl-2-(5H-imidazo[5.1-a]isoindol-5-yl)ethannl (GDC919/NLG919), or (E)-4-Amino-N′-(3-chloro-4-fluorophenyl)-N-hydroxy-1,2,5-oxadiazole-3-carboximidamide (INCB024360).

In some embodiments of this aspect of the disclosure, the cytotoxic agent can be an anthracene selected from the group consisting of doxorubicin, idarubicin, mitoxantrone.

Still another aspect of the disclosure encompasses embodiments of a composition comprising an immunotherapeutic agent effective in generating an immune response directed against a tumor in a recipient patient, a therapeutic agent that elevates the level of protein p53 in a recipient patient, wherein said therapeutic agent is nutlin-3a, and a pharmaceutically acceptable carrier.

In some embodiments of this aspect of the disclosure, the composition can further comprise at least one of an IDO-inhibitor and a cytotoxic agent.

Still another aspect of the disclosure encompasses embodiments of a kit comprising an first therapeutic agent directed against a tumor in a recipient patient, a second therapeutic agent that elevates the level of protein p53 in a recipient patient, and a pharmaceutically acceptable carrier, wherein the first therapeutic agent, the second therapeutic agent, and the pharmaceutically acceptable carrier are packaged individually or in any combination, and instructions for the use of the packaged agents and carrier to prepare an effective dose of each agent for administration individually or in combination to a patient in need thereof.

In some embodiments of this aspect of the disclosure, the therapeutic agent that is capable of elevating the level of protein p53 in a recipient patient is nutlin-3a.

It should be emphasized that the embodiments of the present disclosure, particularly any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Reagents: Human gp10025-33 (KVPRNQDWL, SEQ ID NO: 2) and SIINFEKL (SEQ ID NO: 1) peptides were synthesized by Southern Biotechnology from the published sequence (Hogquist et al., (1994) Cell 76: 17-27; Overwijk et al., (2003) J. Exp. Med. 198: 569-580). VO-OHpic was from Biovision (#1801-5). Whole OVA protein (#A-5503), N-acetyl-L-cysteine (#A-7250) and pifithrin-α (#P-4359)(Komarov et al., 1999) were from Sigma. Nutlin-3a (#18585) was from Cayman Chemical. Indoximod (1-methyl-D-tryptophan, clinical grade) was from NewLink Genetics and was dissolved as described (Hou et al., (2007) Cancer Res. 67: 792-801).

### Example 2

Mouse strains: The following strains were obtained from Jackson Laboratories:

OT-I: (CD8+, recognizing the SIINFEKL peptide of ovalbumin on H2K^{b} (Hogquist et al., (1994) Cell 76: 17-27);

pmel-1: (B6.Cg-Thy1^{a}/CyTg(TcraTcrb)8Rest/J) recognizing a peptide from human gp100 (Overwijk et al., (2003) J. Exp. Med. 198: 569-580);

IL-12p40-KO: (B6.129S1-II 12b^{tm1Jm}/J);

IL-12p35-KO: (B6.129S1-II 12a^{tm1Jm}/J);

Perforin-KO: (C57BL/6-Prf1^{tm1Sdz}/J);

global p53-KO: (B6.129S2-Trp53^{tm1Tyj}/J)(Jacks et al., (1994) Curr. Biol. 4: 1-7);

gp91phox-KO (Cybb-null): (B6.129S-Cybb^{tm1Din})(Pollock et al., (1995) Nat. Genet. 9: 202-209);

RIP-mOVA: (C57BL/6-Tg(Ins2-TFRC/OVA)296Wehi/WehiJ)(Kurts et al., (1996) J. Exp. Med. 184: 923-930);

IFNγ-KO: (B6.129S7-Ifng^{tm1Ts}/J);

The Tg(Grm1)Epv mouse strain (Pollock et al., (2003) Nat. Genet. 34: 108-112) was monitored weekly for development of autochthonous melanoma.

PTEN^{Treg}-KO mice have been described (Sharma et al., (2015) Science Advances 1: e1500845). Bac-transgenic Foxp3^{GFP-Cre} mice (Zhou et al., (2009) Nat. lmmunol. 10: 1000-1007; Zhou et al., (20089) J. Exp. Med. 205: 1983-1991) (NOD/ShiLt-Tg(Foxp3-EGFP/cre)1Jbs/J) were back-crossed onto the B6 background, then crossed with mice bearing a foxed PTEN gene (Lesche et al., (2002) Genesis 32: 148-149) (B6.129S4-Pten^{tm1Hwu}/J). The resulting strain was maintained as hemizygous for Foxp3-GFP-Cre and homozygous for Pten^{loxP/loxP}.

Mice with a nuclear-localizing Cre-recombinase knock-in replacing one copy of the Lyz2 gene (Clausen et al., (1999) Transgenic Res. 8: 265-277) (strain 004781, Jax) were crossed as heterozygotes with homozygous p53^{flox/flox} mice (Marino et al., (2000) Genes Dev. 14: 994-1004) (strain 008462, Jax).

Control mice: Controls for mutant strains were wild-type C57BLJ6J mice. PTEN^{Treg}-KO and LysMcre/p53^{Loxp} mice were inbred on the B6 background, so in validation studies controls were compared using the Cre-expressing parental strain, versus littermates negative for Cre, versus wild-type C57BL/6 mice. All three controls gave comparable results for tumor growth, anti-tumor response, and FACS assays.

### Example 3

Mouse myeloid-cell maturation cultures: Maturation co-cultures were performed in V-bottom wells (Nunc 249952 V96), using RPMI-1640 medium with 10% fetal bovine serum, but no added cytokines. The preferred source of myeloid cells was TDLN or spleen from mice bearing B16F10 or E.G7 tumors. Rapid isolation of cells was important for viability, so cells were disaggregated by passing once through a 40 μm mesh, then stained briefly on ice and FACS-sorted into ice-cold medium using low-shear fluidics and a large nozzle (Mo-Flo cell sorter). Lytic-expressing myeloid cells were enriched by sorting for Ly6c⁺ that were CD11c^{NEG} B220^{NEG} (to exclude dendritic cells and B cells). For some experiments, myeloid cells were sub-fractionated using sort dates based on expression of Gr-1 and CD11b.

Sorted Ly6c+ myeloid cells were added to co-cultures as previously described (Sharma et al., (2013) Immunity 38: 998-1012). As an inflammatory stimulus for differentiation, CD8⁺ effector cells (5×10⁴) were FACS-sorted from spleens of either OT-I or pmel-1 mice (both performed equivalently), and added to co-cultures along with 100 nM cognate peptide (SIINFEKL (SEQ ID NO: 1) or hgp100). It has been shown that during tumor immunotherapy, destabilized Treg cells (“ex-Tregs”) provide an important source of inflammatory helper activity (Sharma et al., (2013) Immunity 38: 998-1012); therefore, Treg cells were sorted from normal B6 spleen as CD4⁺CD25⁺ cells and added at 2×10⁴/well. These were an important source of CD4⁺ help for dendritic cell maturation. All cultures also received a feeder layer of 1×10⁵ T cell-depleted B6 spleen cells (CD4^{NEG}CD8^{NEG}) to maintain T cell viability (Sharma et al., (2007) J. Clin. Invest. 117: 2570-2582). All T cells and feeder layer were made CD45.1⁺ or Thy1.1⁺, so that they could be excluded from analysis after culture.

To ensure that activation was not suppressed by either IDO from the myeloid cells or PTEN expression by the Treg cells, all cultures received inhibitors of IDO (1-methyl-D-tryptophan (indoximod), clinical grade, 200 μM) and PTEN (VO-OHpic, 1 μM). After 3 days, co-cultures were harvested and stained for maturation of the original myeloid population, gated on the congenic markers to exclude other cells.

For mice, tumors, TDLN or spleen were disaggregated and MDSCs enriched by sorting for Ly6c+CD11C^{NEG}B220^{NEG} cells. MDSCs were co-cultured with antigen-specific CD8⁺ T cells, plus cognate antigen and feeder cells, but without added cytokines or growth-factors. Maturation was induced by adding recombinant IFNγ (30 ng/ml). For gene-knockdown studies, siRNA pools were obtained from Santa Cruz Biotechnology, and transfections were performed using the manufacturer's siRNA Reagent Kit. Efficiency of transfection was monitored using FITC-conjugated siRNA tracer, and protein knock-down of each gene was confirmed by FACS. Human MDSC cultures: Human PBMC samples were obtained from Memorial Blood Centers

(Minneapolis, MN) and isolated by centrifugation over Ficoll-Paque PLUS (GE Healthcare), followed by enrichment for myeloid cells using CD33⁺ micro-beads (Miltenyi MACs). Alternatively, myelomonocytic cells were enriched by counter-flow elutriation as previously described (Munn et al., (2002) Science 297: 1867-1870; Munn et al., (2004) J. lmmunol. 172: 4100-4110). Immature myeloid cells were then generated by culture for 7 days as previously described (Koehn et al., (2015) Blood 126: 1621-1628; Lechner et al., (2010) J. lmmunol. 185, 2273-2284). Briefly, PBMC were cultured in RPMI-1640 medium with 10% FBS, 1% Pen/Strep, 1% GlutaMax as well as recombinant human GM-CSF (10 ng/ml, R&D Systems)+IL-6 (10 ng/ml, R&D Systems). Culture growth media was refreshed on days 3 and 6; on day 7, cells were washed and returned to culture in medium without GM-CSF/IL-6. Replicate cultures were then treated for 2 days with or without recombinant human IFNγ (30ng/ml, R&D Systems) to induce maturation, and with or without p53 inhibitor pifithrin-α (10 uM, Sigma). After 48 h, cultures were harvested and stained for CD11b, CD33, phospho-p53-Ser15, IRF5 and BDCA3 (CD141).

### Example 4

siRNA knock-down: siRNA pools were obtained from Santa Cruz Biotechnology against mouse Batf3 (#sc-153654), IRF5 (#sc-72045) and ATM (#sc-29762), or human IRF5 (#sc-72-044), p53 (#sc-29435) and Batf3 (#sc-88553), with associated scrambled control siRNA. Gentle conditions were chosen so as to maximize viability with a goal of achieving approximately 50-60% transfection. Transfected cells were then identified based on uptake of FITC-conjugated tracer oligos (#sc-36869) and showed that mouse Ly6c⁺ myeloid cells took up siRNA in 58±9% of the original population at the end of culture. Human transfection efficiency was similar. Transfection of mouse cells were performed using the manufacturer's siRNA Reagent Kit (#sc-45064). Briefly, Ly6c⁺CD11c^{NEG} myeloid cells were sorted from TDLNs and combined with other cells as for the “M DSC maturation co-cultures” above.

Prior to plating, the mixed cells were suspended in transfection medium and incubated with specific siRNA or control scrambled siRNA diluted in Transfection Reagent. After 6 h at 37° C., 2× growth medium was added (RPMI-1640 with 20% FBS and double the supplements), and cells cultured overnight. The siRNA was not removed during culture. After 24 h, most of the transfection medium was gently aspirated, replaced by 1× culture medium, and cultures continued for an additional 60 h.

Human cells were transfected similarly. Myeloid cells were cultured as described, then siRNA added on day 8. After 6 h at 37° C., most of the transfection medium was gently aspirated and replaced by culture medium with fresh cytokines, and cultures continued for an additional 2 days.

### Example 5

Antigen-presentation assays and rescue of anergy: B16-OVA or E.G7 tumors were harvested following CTX/VO-OHpic immunotherapy, and Ly6c+/CD103+ dendritic cells enriched as follows.

Antigen-presentation: For ex-vivo antigen-presentation assays, dendritic cells were harvested from tumors (rather than TDLNs) so that they would have been directly exposed to tumor antigen. Mice bearing B16-OVA, B16F10, or E.G7 tumors were treated with PTEN-inhibitor immunotherapy (VO-OHpic day 9-14 + CTX day 10). Tumors were harvested on day 14 and disaggregated with collagenase, DNAse, and hyaluronidase as described (Sharma et al., (2015) Science Advances 1: e1500845).

Ly6c+CD103+ dendritic cells were enriched from tumors by sorting for Ly6c⁺CD11c⁺ cells. (The Ly6c⁺CD11c+ population contained all of the Ly6c⁺CD103⁺ dendritic cells, and greater than 80% of Ly6c⁺CD11c⁺ cells were CD103⁺. Ly6c and CD11c were very stable during sorting, whereas CD103 tended to modulate rapidly after staining.) Sorted dendritic cells were added at 5×10³/well plus sorted CD8⁺ effector cells (5×10⁴) from spleens of OT-I mice. Positive control wells included 100 nM SIINFEKL (SEQ ID NO: 1) peptide. (For antigen-presentation assays, the feeder layer and Treg cells used in the maturation cultures were not necessary.) Co-cultures were set up in triplicate. After 3 days, co-cultures were pulsed with ³H-thymidine to measure proliferation.

Anergic T cell rescue: To elicit tumor-induced anergic T cells, congenic OT-I^{Thy1.1} or pmel-1 cells (sorted CD8⁺) were adoptively transferred into B6 hosts, then mice were implanted with bilateral tumors (B16-OVA or B16F10, respectively). After 14 days, the anergic T cells were sorted from TDLNs (Thy1.1⁺CD8⁺). To test for rescue by Ly6c⁺CD103⁺ dendritic cells, activated Ly6c⁺CD103⁺ dendritic cells were prepared in parallel by implanting B16-OVA tumors in different mice, then treating them with PTEN-inhibitor immunotherapy (VO-OHpic day 9-13+CTX day 10).

Ly6c⁺CD103⁺ dendritic cells were enriched from tumors by sorting for Ly6c⁺CD11c⁺ cells, as above; then 5×10³ Ly6c⁺ dendritic cells were co-cultured with 5×10⁴ anergic OT-I cells sorted from TDLNs. (For rescue of anergic T cells, the feeder layer and Treg cells used in the maturation cultures were not necessary.) Co-cultures were set up in triplicate and proliferation measured after 3 days by ³H- thymidine incorporation. Some wells received recombinant IL-12 (R&D systems, #419-ML/CF, 40 ng/ml) or IL-12 neutralizing antibody (anti-IL-12p40, clone C17.8, BioXcell, 1 μg/ml).

DCs were co-cultured with OT-I CD8⁺ T cells for 3 days, and proliferation measured by ³H-thymidine incorporation.

### Example 6

Antibodies and FACS staining: Details of all FACS staining are as follows, including validation of the p53 and phospho-p53 staining antibodies.

For FACS staining, lymph nodes were prepared by rapidly passing through a 40 μm mesh, then stained using short incubation times (10 min on ice), as described (Sharma et al., (2013) Immunity 38: 998-1012). Tumors were disaggregated by treating for 1 h with collagenase, DNAse and hyaluronidase in RPMI 1640 medium, as described (Sharma et al., (2015) Science Advances 1: e1500845).

The following conjugated monoclonal antibodies against mouse antigens were from BD-Bioscience: CD4 (clone RM4-5); CD8α (clone 53-6.7); CD86 (clone GL1); CD11c (clone HL3); Ly6c (clone AL-21); IFNγ (clone XMG1.2); CD24 (clone M1/69). Conjugated antibodies were obtained from eBioscience against: Foxp3 (clone FJK-16s); granzyme B (clone NGZB); PD-1 (Clone: J43); PD-L1 (clone MIH5); CD103 (M290) and Ly6c (clone HK1.4); CD69 (clone H1.2F3); CD11b (clone M1/70); IRF8 (clone V3GYWCH).

For intracellular IL-12 detection antibody against the p40 subunit (clone C17.8) was used because the p35 subunit has been reported to be expressed by many cell types in the tumor microenvironment, without necessarily being secreted; whereas the p40 subunit was associated with cytokine production and was selectively expressed by CD103⁺ dendritic cells (Ruffell et al., (2014) Cancer Cell 26: 623-637). There was no evidence of a role for IL-23, so cross-reactivity with IL-23 was not a concern, and results were confirmed using exogenous recombinant IL-12.

All intracellular antigens except for phospho-p53 were detected using fixation-permeablization reagent and matching perm-wash buffer from eBioscience (Cat. #00-5523), with blocking using 5% normal donkey serum, then acquired immediately after staining. IL-12 and IFNγ were measured after 4 h activation with PMA/ionomycin in the presence of brefeldin A as previously described (Sharma et al., (2013) Immunity 38: 998-1012).

Reactive oxygen species (ROS) were measured using the redox-responsive dye 2′,7′-dichlorofluorescein diacetate (DCFDA) as described (Thevenot et al., (2014) Immunity 41: 389-401).

Antibody against the N-terminus of p53 (clone 1C12), used as in Watanabe et al., (2014) Immunity 40: 681-691, was from Cell Signaling Technology. Antibody against the C-terminus p53 (clone PAb122 (Gurney et al., (1980) J. Virol. 34: 752-763) was from Novus Biologicals. For both antibodies, staining was performed following permeablization with eBioscience fix-and-perm. Anti-phospho-p53(Ser15) (clone D4S1H) was from Cell Signaling Technology. For phospho-specific staining, cells were washed in PBS, fixed with 2% paraformaldehyde for 10 min at 37° C., pre-chilled for 1 min, then permeabilized by slow addition of ice-cold methanol to a final concentration of 90%. Cells were then incubated on ice for 30 min, washed with 1% FCS/PBS, blocked with the same solution for 10 min at room temperature, then for 1 h at room temperature and washed. Cells were acquired immediately after staining.

Validation of p53 staining: It was critical to be able to detect p53 in individual cells by FACS (rather than by bulk Western blot) because many cells in the tumor expressed p53, but only the specific Ly6c⁺CD103⁺ dendritic cell subset was of interest. The antibodies for FACS were clean and specific. However, clone 1C12 and the phospho-specific clone D4S1H both bound near the same residues, and showed stearic hindrance. Therefore, clone Pab122 was used for all dual-staining. To increase rigor, all conclusions from FACS staining were also confirmed by one or more complementary strategies (p53-KO mice, targeted LysMcre/p53^{Loxp} mice, pifithrin inhibitor, or siRNA knockdown of downstream target genes).

### Example 7

In vivo tumor studies: The B16F10 and E.G7 (EL4-OVA) cell lines were obtained from ATCC. B16-OVA (B16F10 transfected with full-length chicken ovalbumin) clone MO4 was according to Falo et al. ((1995) Nat. Med. 1: 649-653). B16F10 cells expressing EGFP protein (B16-GFP cells) were derived using lentiviral transfection. Tumor implantation was performed as described (Sharma et al., (2007) J. Clin. Invest. 117: 2570-2582), using 1×10⁵ cells for B16F10 and 1×10⁶ cells for other cell lines (large inocula were used to ensure rapid tumor engraftment and immune suppression). Tumors were implanted bilaterally in each mouse as a control for reproducible implantation technique; replicate tumor diameters in a single mouse were typically within ±15%. Tumor volume was calculated from orthogonal diameters using the formula V=L×W²×π/6. Mice received approved euthanasia when tumors reached a size of 300 mm² (product of orthogonal diameters); death was never used a planned endpoint in any study.

Tumor implantation was performed as described (Sharma et al., (2007) J. Clin. Invest. 117: 2570-2582). VO-OHpic was given at 10 mg/kg/day i.p., in 10% DMSO/PBS. Cyclophosphamide was given at 150 mg/kg i.p×1 dose; and oxaliplatin at 5 mg/kg i.p×1 dose. Nutlin-3a was administered daily at 10 mg/kg/day i.p. For checkpoint blockade, mice received anti-CTLA-4 plus a cocktail of antibodies against the PD-1 pathway. Vaccines and T cell adoptive transfer studies were performed as described (Sharma et al., (2015) Sci. Advances. 1: e1500845).

### Example 8

VO-OHpic/CTX, nutlin-3a, oxaliplatin and immunotherapy regimens: The vanadate compound VO-OHpic (Mak et al., (2010) J. Chem. Biol. 3: 157-163) inhibits PTEN activity, and this drug was used to destabilize PTEN+Tregs during chemotherapy. Like all of the available PTEN-inhibitors, VO-OHpic can also affect other phosphatases (Spinelli et al., (2015) Adv. Biol. Regul. 57: 102-111). However, it is an effective inhibitor of PTEN in vivo, and it has been shown that when VO-OHpic is combined with chemotherapy, it causes rapid and essentially complete destabilization of PTEN+Tregs in tumors (Sharma et al., (2015) Science Advances 1: e1500845). VO-OHpic is not selective only for Tregs, but the Tregs in tumors appeared particularly dependent on PTEN to maintain their suppressive phenotype. In contrast, effector T cells and antigen-presenting cells expressed little PTEN during activation and showed little effect of PTEN-inhibitor (Sharma et al., (2015) Science Advances 1: e1500845). Thus, with due caveats, VO-OHpic was an effective means of functionally destabilizing PTEN⁺ Tregs during chemotherapy.

VO-OHpic was given at 10 mg/kg/day i.p., in 10% DMSO/PBS. Cyclophosphamide was given at 150 mg/kg i.p×1 dose. Oxaliplatin was given at 5 mg/kg i.p×1 dose. Nutlin-3a was dissolved at 2 mg/ml in 10% DMSO in buffered saline and administered daily at 10 mg/kg/day i.p. The 10 mg/kg/day dose was at the lower end of the parenteral dosing range described for mice (Zhang et al., (2011) Drug Meta. Dispos. 39: 15-21). For checkpoint blockade, mice received anti-CTLA-4 (clone 9D9, BioXcell) plus a cocktail of antibodies against the PD-1 pathway: anti-PD-L2, clone TY25 (Yamazaki et al., (2002) J. Immunol. 169: 5538-5545); anti-PD-1, clone J43 (Agata et al., (1996) Int. Immunol. 8: 765-772); and anti-PD-L1, clone MIH7 (Tsushima et al., (2003) Eur. J. Immunol. 33: 2773-2782). A cocktail was used in order to block the PD-1 pathway as completely as possible and thus minimize any peculiarities of a single blocking antibody.

### Example 9

Ly6c+ MDSC adoptive transfer (intra-tumoral and systemic): For studies of intra-tumoral injection of MDSCs, the approach of Zitvogel & Kroemer ((2014) Cancer Cell 26: 591-593) was modified (Ma et al., (2006) Immunity 38: 729-741). Donor mice (CD45.1 congenic) were implanted with B16F10 tumors, then on day 14 tumors were harvested, disaggregated and intratumoral Ly6c⁺ MDSCs sorted as Ly6c⁺CD11c^{NEG}B220^{NEG} cells. In parallel, recipient mice were implanted with B16F10 tumors 5 days delayed, and the sorted Ly6c⁺ MDSCs were injected into new day 9 recipient tumors (1×10⁶ sorted cells in 50 μl PBS). Recipient mice were then treated with VO-OHpic/CTX immunotherapy and tumors harvested 4 days after transfer.

For systemic adoptive-transfer, the approach of Bronte et al. was modified (Ugel et al., (2012) Cell Repts. 2: 628-639). Immature myeloid cells (Ly6c⁺CD11c^{NEG}CD11b⁺Gr-1⁺) were sorted from the MDSC pool in spleens of tumor-bearing mice. To obtain the maximum yield of MDSCs, donor mice were usually implanted with EL4 tumors for 21 days. It was immaterial which tumor elicited the precursors in donor mice since these were not antigen-specific and EL4 tumors elicited large numbers of MDSCs.

### Example 10

Vaccines and T cell adoptive transfers: For tumor vaccines, human gp100₂₅₋₃₃ was synthesized from the published sequence (Overwijk et al., (2003) J. Exp. Med. 198: 569-580). CpG-1826 (phosphorothioate oligo 5′-TCCATGACGTTCCTGAGCTT-3′) (SEQ ID NO: 3) was synthesized from the published sequence (Chu et al., (1997) J. Exp. Med. 186: 1623-1631) by Tri-link Biotechnologies. Vaccines were prepared with 25 μg peptide and 50 μg CpG-1826 in incomplete Freund's adjuvant (IFA, Sigma F-5506) and administered in the hind-limb footpad. For adoptive transfers, OT-I or pmel-1 spleen cells were enriched by negative selection using magnetic beads (mouse CD8 isolation kit II, #130-095-236, Miltenyi Biotech) or by MoFlo cell sorting using a large-aperture nozzle. Staining for sorting was performed on ice with short incubation times to keep the cells viable but un-activated. Mice received 2×10⁶ enriched CD8⁺ cells via tail-vein.

### Example 11

Statistics: Statistical analysis was performed using GraphPad Prism 7 software. Groups were compared by t-test. Multiple treatment groups were analyzed by ANOVA with Tukey's correction for multiple comparisons. The number of independent replicates for each experiment are indicated in the brief descriptions of the figures. Error bars always show standard deviation

### Example 12

Ly6c+/CD103+ dendritic cells emerge in tumors when PTEN+ Tregs are absent: To study tumors with an inflammatory, immunogenic microenvironment, B16F10 tumors were implanted in host mice with a targeted deletion of PTEN phosphatase in regulatory T (Treg) cells (PTEN^{Treg}-KO mice). It has been shown that tumor-associated Treg cells in these mice become spontaneously unstable and lose their suppressor activity (Sharma et al., (2015) Science Advances 1: e1500845).

Tumors implanted in PTEN^{Treg}-KO mice were immunogenic, chronically inflamed and could barely grow (FIG. 1A). Analysis of these tumors showed a population of CD11c⁺ cells bearing an unusual “hybrid” phenotype (FIG. 1A, dot-plots). These cells expressed Ly6c, suggestive of monocytes or MDSCs (Gallina et al., (2006) J. Clin. Invest. 116: 2777-2790); but they also expressed CD103, suggestive of Batf3-lineage conventional dendritic cells (cDCs) (Satpathy et al., (2012) Nat. Immunol. 13: 1145-1154). These “dual-phenotype” Ly6c⁺/CD103⁺ cells were rare or absent in tumors from wild-type mice but spontaneously appeared when the host lacked the PTEN⁺ Treg population (FIG. 1A, scatter-plot).

Ly6c⁺/CD103⁺ dendritic cells could be elicited by dying tumor cells. When mice lacked PTEN⁺ Tregs, even a single injection of apoptotic tumor cells was sufficient to elicit large numbers of Ly6c⁺/CD103⁺ cells in the draining LNs, whereas in wild-type (WT) hosts no such cells emerged. This suggested that the Ly6c⁺/CD103⁺ population might be actively inhibited by the presence of PTEN⁺ Tregs. To test this, PTEN-sufficient wild-type (WT) Treg cells were adoptively transferred into PTEN^{Treg}-KO hosts, and then implanted tumors. Following challenge with chemotherapy, the mice receiving WT Treg cells showed potent inhibition of the Ly6c+/CD103+ population in tumors.

### Example 13

Ly6c+/CD103+ dendritic cells emerge following immunogenic chemotherapy: One question was whether Ly6c+/CD103+ cells were found in WT hosts if the PTEN+ Tregs could be removed. It has been previously shown that inhibiting PTEN activity by treating mice with the vanadate drug VO-OHpic (Mak et al., (2010) J. Chem Biol. 3: 157-163) renders PTEN⁺ Treg cells in tumors unstable when exposed to inflammation or chemotherapy (Sharma et al., (2015) Science Advances 1: e1500845).

FIG. 1B shows that treatment of WT hosts with VO-OHpic plus even modest doses of cyclophosphamide (CTX) was sufficient to destabilize the PTEN⁺ Tregs and trigger rapid regression of tumors. Analysis of regressing tumors showed large numbers of Ly6c⁺/CD103⁺ cells, comprising up to 60% of total CD11c⁺ cells (FIG. 10). These cells expressed myelomonocytic markers (Ly6c, CD11b and Gr-1 ^{int}) but also expressed CD103 and dendritic markers such as Batf3, IRF8, CD24, XCR1 and Flt3, suggestive of Batf3-lineage conventional dendritic cells (Grajales-Reyes et al., (2015) Nat. Immunol. 16: 708-717). None of the Ly6c⁺/CD103⁺ cells expressed the macrophage-associated marker F4/80. However, they uniformly expressed CD86 and IL-12, suggesting a pro-inflammatory phenotype.

Following chemotherapy, the Ly6c⁺/CD103⁺ population emerged rapidly, within 24 h of the CTX dose. This was prior to any change in tumor size, so Ly6c⁺/CD103⁺ cells were not simply an artifact of late, regressing tumors. Emergence of the Ly6c⁺/CD103⁺ cells required both CTX and VO-OHpic, consistent with the requirement for both drugs to trigger immune activation and tumor regression (compare with FIG. 1B). Prior to treatment, Ly6c⁺/CD103⁺ dendritic cells were low or absent in most tumors (FIG. 10, scatterplot). Untreated tumors often contained “conventional”-appearing CD103⁺ dendritic cells, but these did not express Ly6c or other myeloid markers, and thus were distinct from the Ly6c⁺/CD103⁺ population.

Identical Ly6c⁺/CD103⁺ cells were also found in autochthonous melanoma tumors treated with CTX/VO-OHpic. Further, Ly6c⁺/CD103⁺ cells were not restricted to the CTX/VO-OHpic model: other forms of immunotherapy and immunogenic chemotherapy could spontaneously destabilize PTEN⁺ Tregs; and Ly6c⁺/CD103⁺ dendritic cells were prominent in these models.

### Example 14

Ly6c+/CD103+ dendritic cells can cross-present antigen and rescue anergic T cells: Authentic dendritic cells should be able to acquire antigen and cross-present it to naive T cells. Accordingly, tumor-bearing mice were treated with CTX/VO-OHpic, then Ly6c⁺/CD103⁺ cells (and other putative APC populations) were isolated from the treated tumors. Only the Ly6c⁺/CD103⁺ dendritic cells (but not other dendritic cells or myeloid cells) were able to spontaneously cross-present a nominal antigen acquired in vivo from tumor and activate na [symbol] ve CD8⁺ T cells in vitro.

The Ly6c⁺/CD103⁺ dendritic cells were also the only antigen-presenting cells capable of the demanding task of re-activating T cells rendered anergic (unresponsive) by tumors. For these studies, tumor-specific CD8⁺ T cells were anergized by prolonged exposure to tumor in vivo, rendering them unresponsive to cognate antigen.

In parallel, activated Ly6c⁺/CD103⁺ dendritic cells were sorted from tumors that had been treated with immunotherapy. Only the Ly6c⁺/CD103⁺ dendritic cells (but not other dendritic cells or MDSCs from the same tumor) were able reactivate the anergic T cells (FIG. 1D). This reversal of anergy was dependent on production of IL-12, as shown by addition of neutralizing antibody (FIG. 1D, arrow). Consistent with this, the anti-tumor response to CTX/VO-OHpic therapy was abrogated in mice lacking IL-12, and tumor-associated T cells were unable to become re-activated in vivo.

Thus, taken together, these data indicated that the Ly6c+/CD103+ cells represented authentic dendritic cells and that they can play an important functional role in immune responses following immunotherapy.

### Example 15

Ly6c⁺/CD103⁺ dendritic cells can differentiate in vitro from Ly6c+ immature myeloid cells: The Ly6c⁺/CD103+ cells emerged so rapidly, within 24 h of treatment, that it indicates they can arise from cells already present in the tumor or circulation. The Ly6c⁺, CD11b⁺ and Gr-1^{int} markers were reminiscent of the monocytic MDSCs that can be elicited by tumors (Gabrilovich et al., (2012) Nat. Rev. Immunol. 12: 253-268). Considered in bulk, MDSCs are immunosuppressive; however, some MDSCs resemble immature versions of the inflammatory monocytes and dendritic cells seen in infection (Goldszmid et al., (2014) Cell Host Microbe 15: 295-305; Kumar et al., (2016) Immunity 44: 303-315). To ask whether tumor-elicited immature myeloid cells might give rise to the Ly6c⁺/CD103⁺ dendritic cells, Ly6c⁺CD11c^{NEG} cells were sorted from TDLNs. This bulk population lacked CD103 or other dendritic cell markers, and displayed functional suppressor activity consistent with MDSCs. To provide an inflammatory stimulus for maturation, the Ly6c⁺CD11c^{NEG} cells were co-cultured with effector T cells plus cognate antigen.

Within 24 h, this inflammatory exposure drove a portion of the Ly6c⁺ cells to differentiate into CD11c⁺ cells co-expressing CD103 and Ly6c. Further sub-fractionation of the original immature population indicated that the specific precursors for the mature Ly6c⁺/CD103⁺ cells were the Gr-1⁺CD11b⁺ subset of Ly6c⁺ cells.

### Example 16

Differentiation of Ly6c+/CD103+ cells requires Batf3: Mature Ly6c⁺/CD103⁺ cells up-regulated multiple markers suggestive of conventional CD103+ dendritic cells: a differentiation program that would normally be controlled by the transcription factor Batf3 (Satpathy et al., (2012) Nat. Immunol. 13: 1145-1154).

It was hypothesized that Batf3 might become ectopically activated in Ly6c⁺ cells in response to inflammation. FIG. 2A shows that the immature Ly6c+CD11c^{NEG} cells up-regulated Batf3 during differentiation in vitro, and knock-down of Batf3 abrogated acquisition CD103 and associated markers (IRF8, CD24 and IL-12). Consistent with the hypothesis, Batf3-deficient mice (Hildner et al., (2008) Science 322: 1097-1100) treated with CTX/VO-OHpic were unable to generate the Ly6c⁺/CD103⁺ dual-positive dendritic cell population, even though they possessed other tumor-associated CD11c⁺ cells. In other studies, it was found that immature Ly6c⁺ cells up-regulated high levels of the FLT3 receptor during maturation and became strictly dependent on FLT3-ligand in order to acquire the CD103+ phenotype in vitro. Thus, the “MDSC-like” precursor population activated a Batf3- and FLT3-dependent “DC-like” differentiation program during maturation.

### Example 17

Immature precursors for Ly6c+/CD103+ cells are found in tumors: To determine if immature Ly6c+ precursor cells could be found in tumors, tumor-resident Ly6c⁺CD11c^{NEG} cells were isolated from untreated B16F10 tumors by FACS-sorting, then injected directly into new tumors in different hosts. If the recipient mice received no further treatment, then the injected cells remained immature and did not acquire CD103 or other dendritic cell markers. However, when recipient mice were treated with CTX/VO-OHpic, then many of the transferred cells differentiated into mature Ly6c⁺/CD103⁺ dendritic cells that were phenotypically identical to the endogenous Ly6c⁺/CD103⁺ dendritic cells of the host.

To determine whether the Ly6c⁺/CD103⁺ cells could acquire antigen from tumors, immature Ly6c⁺ cells were injected into tumors expressing a fluorescent GFP marker. Following treatment with CTX/VO-OHpic, the transferred cells matured into Ly6c⁺/CD103⁺ dendritic cells, took up tumor-derived GFP protein, and transported it to the draining LNs. This, taken together with the ability of these cells to acquire and cross-present tumor-derived antigens to T cells (FIG. 1D), suggested that the Ly6c⁺/CD103⁺ cells behaved as authentic dendritic cells.

### Example 18

Monocyte-lineage progenitor cells (cMoPs) in bone marrow can give rise to Ly6c⁺/CD103⁺ dendritic cells: Classically, Batf3-dependent dendritic cells should arise from committed dendritic cell progenitor cells (CDP) in the bone marrow (Liu et al., (2009) Science 324: 392-397), not from Ly6c⁺ cells. To test this, dendritic-lineage CDPs were isolated from bone-marrow (Ly6c^{NEG} CD115⁺ CD117⁺ CD135⁺). From the same bone marrow committed monocyte-lineage precursor cells (“cMoPs”) were also isolated that were Ly6c⁺CD115⁺CD117⁺CD135^{NEG} (Hettinger et al., (2013) Nat. Immunol. 14: 821-830).

CDPs and cMoPs were then transferred separately into congenically-marked hosts bearing established tumors, and all recipients treated with CTX/VO-OHpic (FIG. 2B). Following treatment, many of the monocyte-lineage cMoP cells differentiated into Ly6c⁺/CD103⁺ dendritic cells in tumors, up-regulating CD11c, Batf3 and CD103 expression, while retaining Ly6c. In contrast, although dendritic-lineage CDPs could home to tumors and express some of the same dendritic cell markers, they showed little CD103 (which often is not expressed by conventional cDCs (Satpathy et al., (2012) Nat. lmmunol. 13: 1145-1154), and none of these acquired Ly6c (FIG. 2B scatter-plots). Thus, the dual-positive Ly6c⁺/CD103⁺ population appeared to arise specifically from monocytic rather than dendritic-lineage precursors.

### Example 19

Monocytic progenitor cells rescue anti-tumor activity in Batf3-deficient mice: Batf3-deficient mice lack critical dendritic cell activity required for anti-tumor immunity (Broz et al., (2014) Cancer Cell 26: 638-652; Hildner et al., (2008) Science 322: 1097-1100; Spranger et al., (2017) Cancer Cell 31: 711-723.e714).

Although the expression of Batf3 by Ly6c⁺/CD103⁺ cells was non-canonical, it was determined whether these cells could functionally rescue immune responses in Batf3-deficient mice. As expected, Batf3-KO mice showed essentially no anti-tumor effect when treated with CTX/VO-OHpic (FIG. 2C), which is strictly immune-dependent (Sharma et al., (2015) Science Advances 1: e1500845). Adoptive transfer of classical dendritic precursors (VVT CDPs) had only modest effect, with tumors showing some growth-delay but no regression. However, transfer of VVT cMoP cells fully restored anti-tumor activity with rapid tumor regression (FIG. 2C). Anti-tumor activity was likewise restored by transfer of spleen-derived Ly6c⁺CD11c^{NEG} Gr-1⁺CD11b⁺MDSCs. Thus, in this model, the monocytic-lineage Ly6c⁺/CD103⁺ dendritic cells appeared to be a critical site of Batf3 expression.

The Ly6c⁺/CD103⁺ dendritic cells could arise from either bone-marrow cMoPs or peripheral Ly6c⁺CD11b⁺Gr-1⁺ MDSCs. Thus, these two cell types might be closely related. Extended phenotyping showed that the two cell types were essentially indistinguishable, with both populations being Ly6c⁺CD115⁺CD117⁺Gr-1⁺ and variable CD11b.

### Example 20

The transcription factor p53 controls differentiation of Ly6c+/CD103+ dendritic cells: It was determined which regulatory factors might control the maturation step into Ly6c+/CD103+ dendritic cells. Exploratory analysis of genes associated with immunity, inflammation and senescence unexpectedly revealed that the Ly6c⁺/CD103⁺ dendritic cells expressed several known targets of the transcription factor p53 (e.g., p21^{Waf1}, p16^{INK4a} and IRF5). In the immune system, p53 has generally been associated with tolerance and immune suppression (Munoz-Fontela et al., (2016) Nat. Rev. Immunol. 16: 741-750), but in certain settings p53 can also drive a pro-inflammatory program in macrophage-lineage cells (Lowe et al., (2014) Cancer Res. 74: 2182-2192; Slatter et al., (2016) Oncolmmunology 5: e1112941; Jayadev et al., (2011) Glia 59: 1402-1413; Lujambio et al., (2013) Cell 153: 449-460; Pribluda et al., (2013) Cancer Cell 24: 242-256).

Prior to immunotherapy, immature Ly6c⁺CD11c^{NEG} cells in tumors uniformly expressed p53 protein (FIG. 3A), but they did not show phosphorylation at Ser15 that might suggest functional activation (Loughery et al., (2014) Nucleic Acids Res. 42: 7666-7680). (p53 has multiple phosphorylation sites, but in these studies Ser15 was followed as one hallmark of p53 activation.) However, when mice were treated with CTX/VO-OHpic, a distinct population of cells acquired Ser15 phosphorylation, corresponding closely to the mature Ly6c⁺/CD103⁺ dendritic cells (FIG. 3A). Phosphorylation occurred rapidly, within 24 h of treatment, and required both CTX and VO-OHpic.

To ask whether p53 was mechanistically important, tumors were grown in p53-deficient hosts (p53-KO) and treated with CTX/VO-OHpic (FIG. 3B). p53-KO mice lacked the specific Ly6C⁺/CD103⁺ dendritic cell population in tumors. Other CD11c⁺ cells were present, but the Ly6C⁺/CD103⁺ dual-positive cells were absent. p53-KO mice had ample numbers of MDSCs, as previously reported (Guo et al., (2013) Cancer Res. 73: 1668-1675), so the defect was not a lack of immature myeloid cells. However, when Ly6c⁺CD11c^{NEG} cells from p53-KO mice were tested in vitro, they were unable to mature into Ly6C⁺/CD103⁺ dendritic cells (FIG. 3C).

Based on this, it was asked what differentiation signals might be dependent on p53. Batf3 itself is not a known target of p53. However, the transcription factor IRF5 is a direct target of p53 (Mori et al., (2002) Oncogene 21: 2914-29182), and IRF5 is an important regulator of dendritic cell maturation (Lazzari & Jefferies (2014) Clin. Immunol. 153: 343-352).

Following CTX/VO-OHpic treatment, it was found that IRF5 expression corresponded closely with p53-Ser15 phosphorylation in the dendritic cells; and that p53-deficient mice lost all IRF5 expression (FIG. 3D). Functionally, siRNA knock-down of IRF5 during in vitro differentiation markedly inhibited maturation of the CD103⁺ dendritic cell population, and prevented upregulation of Batf3 expression (FIG. 3E). Thus, taken together, these data indicate that p53 was required to induce IRF5, and then the p53-IRF5 pathway led to activation of Batf3 and resulting differentiation.

### Example 21

p53 activation is triggered by the myeloid respiratory burst: Although p53 was clearly important, it was unclear how signals from the immune system could activate the p53 pathway. The classic trigger for p53 activation would be DNA damage, acting via the ATM kinase (Kruiswijk et al., (2015) Nat. Rev. Mol. Cell Biol. 16: 393-400). However, ATM can also be directly activated by reactive oxygen species (ROS) (Guo et al., (2010) Science 330: 517-521); and ROS are known to act as signaling molecules in myeloid cells (Finkel T. (2011) J. Cell Biol. 194: 7-15).

Using the in vitro differentiation model, it was found that Ly6c+CD103⁺ dendritic cells up-regulated high levels of ROS during differentiation in an IFNγ-dependent fashion (FIG. 4A). The source of IFNγ was established to be the antigen-activated effector T cells. This inducible respiratory burst was much higher than the low-level ROS produced constitutively by the MDSCs (FIG. 4A, top histogram) (Thevenot et al., (2014) Immunity 41: 389-401). In myeloid cells, a major source of inducible ROS is the gp91phox system (Cybb). When tumors were grown in Cybb-null mice and treated with CTX/VO-OHpic, the ROS-deficient mice were unable to phosphorylate p53 in Ly6c⁺ cells (FIG. 4B). Consistent with this, downstream IRF5 was not induced in the absence of gp91phox, the Batf3 pathway was not activated, and the Ly6C⁺/CD103⁺ dendritic cell population failed to differentiate (FIG. 4C).

Support for the hypothesis that ATM kinase acted as the redox sensor for the respiratory burst was that siRNA knock-down of ATM in vitro substantially reduced p53 phosphorylation, decreased expression of IRF5, and blocked maturation of immature myeloid cells into Ly6c⁺CD103⁺ dendritic cells (FIG. 4D).

In addition to ROS, the Ly6C⁺/CD103⁺ dendritic cells also expressed the nitric-oxide synthase NOS2. This finding links the inflammatory Ly6C⁺/CD103⁺ dendritic cells to the NOS2⁺ myeloid dendritic cells (“Tip-DCs”) (Marigo et al., (2016) Cancer Cell 30: 377-390). Although the experimental systems differ, and no role for p53 was suggested in that system, it is possible that immunotherapy-induced Tip-DCs reflect a similar pathway of immunogenic myeloid dendritic cell differentiation in inflamed tumors.

### Example 22

A maturation pathway in human myeloid cells is controlled by p53 and IRF5: It was determined whether human cells possessed a p53-mediated maturation pathway analogous to the mice. While in vitro culture systems do not recapitulate authentic differentiation of real dendritic cells, they were a useful model to ask whether human myeloid cells possessed a maturation pathway driven by the same characteristic p53-IRF5-Batf3 cascade.

Human peripheral-blood mononuclear cells were enriched for monocytic cells, then cultured in GM-CSF+IL-6 as described by Koehn et al. ((2015) Blood 126: 1621-1628). Many of the resulting cells expressed the myeloid marker CD33⁺, but they lacked maturation markers CD83, CD141, XCR1 or Batf3 (FIG. 5A). However, when these cultures were treated with recombinant IFNγ (a maturation stimulus analogous to the mouse co-cultures), the CD33⁺ cells rapidly matured into activated cells expressing CD83, CD141, XCR1 and Batf3 (scatter-plot, FIG. 5A).

Using this model, it was determined whether the maturation step was dependent on p53. Addition of siRNA against p53 effectively knocked down p53 expression in cells that were transfected, and this entirely abrogated up-regulation of maturation markers CD83, CD141 and XCR1 (FIG. 5B). During maturation, the CD33⁺ cells up-regulated IRF5 and Batf3 (just as in the mice) and both these downstream transcription factors were lost in the absence of p53. Individual siRNA knock-down of either IRF5 or Batf3 likewise prevented myeloid cell maturation (FIGS. 5C and 5D). Thus, in this in vitro model, the p53-IRF5-Batf3 pathway seen in the mice also appeared relevant to the human cells.

### Example 23

Targeted deletion of p53 in myeloid cells ablates the Ly6C⁺/CD103⁺ dendritic cell population: To ask whether p53 controlled the differentiation of Ly6C⁺/CD103⁺ cells in vivo, mice with a targeted deletion of p53 in myelomonocytic cells were used. To target these cells, cre-recombinase driven by the Lyz2 promoter (Clausen et al., (1999) Transgenic Res. 8: 265-277) was selected. LysMcre is expressed in many monocyte/macrophage cells, including Ly6c⁺ monocytes (Gamrekelashvili et al., (2016) Nat. Commun. 7: 12597). To ask whether the construct was expressed specifically in the cells that gave rise to Ly6c⁺/CD103⁺ dendritic cells, fate-mapping studies were performed using LysMcre crossed to ROSA26-STOP-flox-YFP reporter mice. This irreversibly marks cells that have expressed LysMcre during development.

In untreated tumors (FIG. 6A, upper panels), the few CD103⁺ dendritic cells were all conventional cDCs (no Ly6c expression) and these did not mark with the LysMcre-YFP reporter. However, following treatment with CTX/VO-OHpic, the majority of CD103⁺ dendritic cells in tumors were now dual-positive Ly6C⁺/CD103⁺, and the majority of these marked with LysMcre-YFP (FIG. 6A, lower panels). Likewise, analysis of the immature Ly6c⁺ cells in TDLNs showed that the population of cells marking with LysMcre-YFP were specifically the CD11b⁺Gr-1⁺ subset (which were the cells able to give rise to Ly6C⁺/CD103⁺ dendritic cells).

The LysMcre promoter did not label all of the Ly6C⁺/CD103⁺ cells. It is known that LysMcre is not expressed in all monocytic cells, and only 50-60% of Ly6c⁺ monocytes will mark with this reporter system (Gamrekelashvili et al., (2016) Nat. Commun. 7: 12597). Nevertheless, these fate-mapping studies showed that a major transformation occurred during immunotherapy: from a baseline in which none of the CD103⁺ dendritic cells were derived from the myelomonocytic lineage, to the majority of CD103⁺ dendritic cells being so derived following treatment.

Based on this, it was determined whether ablating p53 in the LysMcre-expressing cells could alter maturation of Ly6C⁺/CD103⁺ dendritic cells. LysMcre mice crossed with mice expressing a targeted p53flox/flox gene (LysMcre/p53^{Loxp} mice) appeared normal and healthy but showed an almost complete absence of p53 in CD11b⁺ myeloid cells in tumors. When tumors were grown in LysMcre/p53^{Loxp} mice and treated with CTX/VO-OHpic, the knockout mice showed a marked reduction in the Ly6C⁺/CD103⁺ population (FIG. 6B). This change was selective for the Ly6C⁺/CD103⁺ subset of dendritic cells: the overall number of CD11c⁺ cells was comparable to WT controls (FIG. 6B, upper scatter-plot) as was the number of “conventional” of CD103⁺ dendritic cells (i.e., not co-expressing Ly6c). However, the specific Ly6C⁺/CD103⁺ population was lost.

It was predicted that this loss should be due to a cell-intrinsic defect in the ability of Lyc6⁺ precursors to mature in response to inflammation. To test this, precursor Ly6c⁺CD11c^{NEG}CD11b⁺Gr-1⁺ MDSCs were sorted from spleens of tumor-bearing mice and transferred into WT mice with tumors. Recipients were then treated with immunogenic chemotherapy (FIG. 6C). For these studies splenic MDSCs were used rather than bone-marrow cMoPs because the LysMcre promoter is not yet expressed in the immature cMOPs (Gamrekelashvili et al., (2016) Nat. Commun. 7: 12597). Ly6c⁺ cells from LysMcre/p53^{Loxp} donors could home to tumors, but they did not express p53 (FIG. 6C, FACS plot) and were unable to mature in response to treatment (i.e., did not up-regulate Batf3, IRF8 or CD24). In contrast, within the same tumor, the endogenous WT dendritic cells up-regulated these markers normally (FIG. 6C, scatterplots) and matured into Ly6C⁺/CD103⁺ dendritic cells. Thus, taken together, Ly6c⁺ precursor cells from LysMcre/p53^{Loxp} mice showed a cell-intrinsic inability to mature into Ly6C⁺/CD103⁺ dendritic cells.

Of note in these adoptive-transfer studies, the LysMcre promoter was efficient at deleting p53 in those Ly6c⁺ cells that reached the tumor. In this inflammatory environment, the penetrance of the LysMcre promoter appeared to be high.

Although the defect in the LysMcre/p53^{Loxp} mice affected only a small subset of dendritic cells, the loss of this population had a profound impact on anti-tumor immunity (FIG. 6D). LysMcre/p53^{Loxp} hosts lost their ability to mount an anti-tumor response following CTX/VO-OHpic treatment with tumors growing unchecked instead of shrinking. This defect was specifically due to the absence of Ly6C⁺/CD103⁺ dendritic cells because adoptive-transfer of WT precursor cells (Ly6c⁺CD11c^{NEG}Gr-1⁺CD11b⁺ cells) fully rescued anti-tumor activity (FIG. 6E)

LysMcre/p53^{Loxp} mice were also unable to respond immunotherapy using a potent regimen of T cell adoptive-transfer, vaccination and dual checkpoint-blockade. Even with this aggressive multi-modal treatment, LysMcre/p53^{Loxp} mice could not generate Ly6C⁺/CD103⁺ dendritic cells in the tumor; the pmel-1 T cells were suppressed; and the tumors grew progressively despite therapy (FIG. 6F).

Similarly, using an established model of immunogenic cell death (ICD) in which E.G7 tumors are treated with oxaliplatin chemotherapy (Ghiringhelli et al., (2009) Nat. Med. 15: 1170-1178), the LysMcre/p53^{Loxp} mice lost all response to treatment. Immunogenic cell death is known to be driven by inflammatory signals such as TLR4 and ATP receptors (Apetoh et al., (2007) Nat. Med. 13: 1050-1059; Ghiringhelli et al., (2009) Nat. Med. 15: 1170-1178), and the Ly6C⁺/CD103⁺ dendritic cells proved to be the main cells that expressed these receptors following oxaliplatin treatment.

Thus, the LysMcre/p53^{Loxp} mice demonstrated that disrupting p53-dependent differentiation of myeloid-lineage Ly6C⁺/CD103⁺ dendritic cells created a profound functional defect in the ability to mount an anti-tumor immune response.

### Example 24

Therapeutic augmentation of Ly6C⁺/CD103⁺ dendritic cells using a p53-agonist drug: Since myeloid p53 thus appeared to be a key component of the response to immunotherapy, it was determined whether increasing the level of p53 could enhance the immune response. In many cells, the level of p53 is controlled by the ubiquitin-ligase MDM2; thus, drugs that inhibit MDM2 will increase the level of p53 in these cells. MDM2-inhibitor drugs currently in clinical trials tended to increase p53 expression in tumors (Khoo et al., (2014) Nat. Rev. Drug Discov. 13: 217-236).

It was found that many mononuclear cells in tumors constitutively expressed p53 with the highest expression being in CD11c⁺ cells and myeloid cells. Systemic treatment with the MDM2-inhibitor drug nutlin-3a (Vassilev et al., (2004) Science 303: 844-848) caused a 2-3-fold increase in p53 levels in these cells, specifically including the Ly6c⁺CD11c^{NEG} myeloid precursors (FIG. 7).

To determine whether this would enhance immune responses, a checkpoint-blockade regimen (FIG. 8A) was employed. By itself, this regimen had only modest effect against established B16F10 tumors. However, the addition of nutlin-3a significantly enhanced and prolonged the effect of treatment. When examined after 2 weeks of treatment, the tumors treated with checkpoint blockade alone were rapidly re-growing and had lost all Ly6c⁺/CD103⁺ dendritic cells; while the addition of nutlin maintained large numbers of Ly6C⁺/CD103⁺ dendritic cells (FIG. 8A, dotplots), and the tumors were still small, inflamed and regressing.

Nutlin-3a showed a similar beneficial effect when combined with CTX/VO-OHpic (FIG. 8B). CTX/VO-OHpic is a more potent regimen, and treated tumors initially shrank with or without nutlin. However, without nutlin the tumors regrew when immunotherapy was stopped; whereas adding nutlin markedly prolonged and maintained the anti-tumor effect.

Likewise, adding nutlin-3a enhanced response to immunogenic chemotherapy (E.G7 tumors treated with oxaliplatin, FIG. 8C). Treatment with nutlin maintained long-term expression of the immunogenic Ly6C⁺/CD103⁺ dendritic cells in tumors and significantly increased the intensity and duration of the anti-tumor effect compared to oxaliplatin alone.

Finally, to determine whether the activity of nutlin was due to an on-target effect on myeloid-lineage p53, responses in LysMcre/p53^{Loxp} KO mice were compared. In mice lacking myeloid p53, nutlin-3a lost all ability to enhance anti-tumor immunity (FIG. 8D). The same result was seen using the CTX/VO-OHpic regimen and also using checkpoint blockade therapy. Thus, the immune-enhancing activity of nutlin was an on-target effect on myeloid-lineage p53.

### Example 25

The immune-activating effect of p53 is context-specific for the tumor milieu: The immunogenic effect of nutlin-3a seemed paradoxical because in most settings p53 is thought to be immunosuppressive (Munoz-Fontela et al., (2016) Nat. Rev. lmmunol. 16: 741-750; Thomasova et al., (2012) Neoplasia 14: 1097-1101). It was considered that p53 actually plays two roles in the immune system: a self-tolerance role in normal tissues during physiologic cell death (Yoon et al., (2015) Science 349: 1261669); but a pro-inflammatory role in certain specialized contexts such as immune-surveillance of tumors (Munoz-Fontela et al., (2016) Nat. Rev. lmmunol. 16: 741-750). To test this, a model in which OVA-expressing tumors were implanted in RIP-mOVA mice (which constitutively express OVA in pancreatic islet cells) was used. OVA thus became both a normal tissue antigen and a tumor antigen.

When the mice were treated with OVA-specific T cells plus CTX/VO-OHpic the tumors shrank, but the mice rapidly succumbed to lethal autoimmune diabetes. Adding nutlin-3a to the treatment was able to selectively suppress OVA-specific T cell responses outside of the tumor, and thus protected the mice from lethal autoimmunity. However, inside the tumor milieu, where inflammation was driven by Ly6C⁺/CD103⁺ dendritic cells, nutlin increased the activation of the same T cells, against same antigen, and markedly enhanced the anti-tumor response (FIG. 13). This “context-specific” immune effect p53 was likewise seen when nutlin-3a was added to checkpoint-blockade immunotherapy, using the same RIP-mOVA model; and also with immunogenic oxaliplatin therapy. Thus, nutlin-3a and p53 showed a surprising ability to promote immune responses to a shared-self antigen in the context of the tumor while actively suppressing autoimmune responses against the same antigen in the context of normal tissues.

### Example 26

Although certain chemotherapy drugs in certain tumors can trigger immunogenic cell death, in most cases dying tumor cells behave as if they were tolerogenic rather than immunogenic, and the immune system remains inert. In addressing this problem, a potent and previously unsuspected immunosuppressive mechanism in the tumor microenvironment has been identified, mediated by the PTEN phosphatase pathway in Tregs. Genetic knockout and pharmacologic inhibition of this pathway reveal that PTEN-expressing Tregs (PTEN-Tregs) function in the immune system as a fundamental mechanism of tolerance to apoptotic cells.

In tumors, this pathway is required in order for tumors to create their normal immunosuppressive microenvironment; and in mice lacking this pathway, tumors become highly immunogenic, constantly inflamed, and can barely grow. It has now been shown that one of the main immunosuppressive effects of these PTEN-Tregs is to force the tumor-associated monocytic MDSC population to remain arrested at an immature, suppressive stage, and prevent their differentiation into highly inflammatory, immunogenic dendritic cells.

When the PTEN pathway is blocked by, for example, administering pharmacologic

PTEN-inhibitor drugs at the time of chemotherapy, then dying tumor cells now trigger rapid differentiation of MDSCs into mature myeloid-lineage dendritic cells. These dendritic cells continue to express myeloid markers, but they acquire expression of CD103, IRF8, and Zbtb46; up-regulate IL-12; and can potently cross-present tumor-derived antigens to CD8+ T cells. These Ly6c⁺CD103⁺ dendritic cells (but no other myeloid cells in the tumor) are selectively lost in Batf3-KO mice; maturation of this specialized subset of dendritic cells (and no other) is potently inhibited by PTEN-Tregs. While not wishing to be bound by any one hypothesis, it is considered that maturation of these dendritic cells is critically important for immune surveillance because they are uniquely able to reverse the anergic state of CD8+ effector T cells in the tumor, and drive a robust, self-amplifying cascade of anti-tumor immunity (as schematically shown in FIG. 9).

PTEN is a readily “drug-able” target, and inhibitor drugs for this novel immune checkpoint are in active development. Further, the findings implicate host p53 as a novel target for immune modulation, with a completely new and powerful mechanism of action, which becomes active when PTEN-Tregs are inhibited.

A fundamental problem is that the antigens released by chemotherapy are not cross-presented by the right kind of antigen-presenting cells (APCs). Following most chemotherapy, tumor antigens are presented by the incompetent and tolerizing dendritic cells found in tumors rather than by the highly activated, inflammatory dendritic cells that are needed activate a robust immune response. This problem impacts more than just chemotherapy: conventional immunotherapy approaches also kill tumor cells and release antigens; but, as with chemotherapy, these endogenous antigens usually fail to trigger a robust response from host T cells. Current attempts to address these defects by blocking the PD-1 or CTLA-4 pathways (Bezu et al., (2015) Front lmmunol. 6: 187) have not been notably successful. This is not surprising because PD-1 and CTLA-4 are expressed on T cells, which is too far downstream. It does not address the real problem, however, which is that the antigen-presenting cells themselves, and the whole associated inflammatory milieu, are all profoundly defective.

To address this key problem, a new molecular target, PTEN phosphatase in Tregs was identified. PTEN-Tregs control a critical upstream checkpoint that acts very early, immediately after tumor cell death (Sharma et al., (2015) Science Advances 1:e1500845). There is mutually-reinforcing evidence (Sharma et al., (2015) Science Advances 1:e1500845; Huynh et al., (2015) Nat. lmmunol. 16: 188-196; Shrestha et al., (2015) Nat. lmmunol. 16: 178-187) that PTEN-Tregs constitute a fundamental mechanism in the immune system that helps create tolerance to apoptotic cells. In the tumor, these PTEN-Tregs potently block the differentiation of a critical population of immunogenic, cross-presenting Ly6c⁺CD103⁺ myeloid dendritic cells. These Batf3-dependent dendritic cells, which are closely similar to immunogenic dendritic cells in tumors (Hildner et al., (2008) Science 322: 1097-1100; Broz et al., (2014) Cancer Cell 26: 638-652; Spranger et al., (2015) Nature 523: 231-235; Sanchez-Paulete et al., (2016) Cancer Discovery 6: 71-79), only emerge in large numbers when the PTEN-Tregs are blocked. These dendritic cells are crucial because they cross-present tumor antigens in an immune-activating fashion and can reactivate anergic anti-tumor T cells for immune surveillance. Thus, when inhibition by PTEN-Tregs is removed such as by administering a PTEN-inhibitor drug during chemotherapy, the whole immunologic microenvironment in the tumor undergoes a radical transformation.

Once the suppression by PTEN-Tregs is removed, conventional chemotherapy now becomes able to activate a whole new set of potent immune-mediated killing mechanisms which would not otherwise be enlisted. Essentially, the combination becomes a totally different drug with new molecular mechanisms. It creates authentic synthetic lethality (Kaelin W G, Jr., Nat. Rev. Cancer 5: 689-698). The same is potentially true for immunotherapy agents as well because the same suppressive PTEN-Tregs also inhibit activation of the host immune response against endogenous tumor antigens (epitope spreading), which is critical for maximum effect of conventional immunotherapy (Chen & Mellman (2013) Immunity 39: 1-10). Thus, blocking the PTEN pathway has the potential for high-impact mechanistic synergy with both chemotherapy and existing immunotherapy.

An approach to Tregs: destabilize and reprogram: Many strategies have been proposed to deplete Tregs in tumors, but none have met with great success (Rech et al., (2012) Sci. Transi. Med. 4: 134ra62; Sugiyama et al., (2013) Proc. Natl. Acad. Sci. USA. 110: 17945-17950; Mitchell et al., (2011) Blood 118: 3003-3012). The novel approach of the disclosure does not try to physically remove the Tregs, but to destabilize them by blocking PTEN and allow them to naturally reprogram into inflammatory helper cells. As previously shown (Sharma et al., (2013) Immunity 38: 998-1012; Sharma et al. (2010) Immunity 33: 942-954), these “ex-Tregs” are a potent source of CD40-ligand and IL-2, and they provide important helper activity for anti-tumor immunity. Since this reprogramming is part of the normal physiology of Tregs in an inflammatory setting (Sharma et al., (2013) Immunity 38: 998-1012; Sharma et al. (2010) Immunity 33: 942-954; Bailey-Bucktrout et al., (2013) Immunity 39: 949-962; Lee Jee et al., (2015) Immunity 42: 1062-1074; Komatsu et al., (2014) Nat. Med. 20: 62-68; Laurence et al., (2012) Immunity 37: 209-222), a natural immune pathway is facilitated. Importantly, the Tregs in tumors are dependent on PTEN for their suppressor activity, whereas most of the Tregs in normal tissues are not. Thus, those Tregs on which the tumor most depends are preferentially destabilized.

When treated with chemotherapy +PTEN-inhibitor, apparently anergic and exhausted CD8 T cells in tumors are found to be potently cytotoxic resident-memory T cells (T_{RM}); while the suppressive monocytic MDSCs in tumors are actually the precursor cells for highly immunogenic dendritic cells (Ly6c⁺CD103⁺ dendritic cells). When immune surveillance is freed from its inhibition by PTEN-Tregs, the anti-tumor effect of the immune system can be far more powerful than the actual chemotherapy that triggered it. This new view of a constant, powerful immune surveillance barely held in check by PTEN-Tregs represents a major paradigm shift for understanding of chemo-immunotherapy.

Maturation of Ly6c⁺CD103⁺ dendritic cells from MDSCs: The data of the disclosure reveal an important role for a novel population of Ly6c⁺CD103⁺ dendritic cells in re-activating immune surveillance. These dendritic cells produce IL-12 and are dependent on the Batf3 transcription factor, and thus are fully consistent with the literature on Batf3+ cross-presenting dendritic cells in tumors, which are known to be important in immune surveillance (Broz et al., (2014) Cancer Cell 26: 638-652; Broz and Krummel (2015) Cancer lmmunol. Res. 3: 313-319). Unexpectedly, however, when PTEN-Tregs are blocked, these Ly6c⁺CD103⁺ dendritic cells arise via direct maturation of immature, suppressive monocytic MDSCs.

While the ability of MDSCs to undergo terminal maturation is described (Ma et al., (2013) Immunity 38: 729-741; Koehn et al., (2015) Blood 126: 1621-1628), it had not been previously known that they can differentiate into Batf3-lineage dendritic cells. However, while dendritic cells in general show substantial plasticity (Paul et al., (2014) Curr. Opin. lmmunol. 30: 1-8), this novel pathway from MDSCs to CD103⁺ dendritic cells is normally strongly inhibited by PTEN-Tregs, so it has not been previously appreciated. Once allowed to differentiate, however, this new dendritic cell population plays a central role in re-activating T cells in the tumor and triggering immune surveillance.

Role for p53 in the immune system: The differentiation step from MDSCs to Ly6c⁺CD103⁺ dendritic cells is controlled by the transcription factor p53. This novel discovery shows that p53 plays two fundamentally different roles in the immune system. In normal tissues p53 promotes self-tolerance and immunosuppression (Yoon et al., (2015) Science 349: 1261669; Watanabe et al., (2014) Immunity 40: 681-691); whereas in the tumor microenvironment it is now shown that p53 orchestrates inflammation and immune surveillance. This profound context-specific difference allows us to propose the high-impact hypothesis that systemic activation of p53 (using clinically-relevant p53-agonist MDM2-inhibitor drugs) is able to selectively suppress autoimmune responses in normal tissues, while actively enhancing beneficial immune responses in the tumor, even against the same antigens. No previous molecular mechanism displays this remarkable context-specific dual therapeutic property.

PTEN-Tregs are a fundamental mechanism of tolerance to apoptotic cells. Although PTEN-Tregs have only recently been discovered, it has been shown that they represent a fundamental pathway in the immune system because they enforce tolerance to dying self cells (Sharma et al., (2015) Science Advances 1:e1500845; Huynh et al., (2015) Nat. lmmunol. 16: 188-196; Shrestha et al., (2015) Nat. lmmunol. 16: 178-187). This is critical to prevent autoimmunity, and mice lacking PTEN in Tregs spontaneously develop lupus as they age (Sharma et al., (2015) Science Advances 1:e1500845; Huynh et al., (2015) Nat. lmmunol. 16: 188-196). These mice immediately lose self-tolerance if they are challenged with a wave of apoptotic cells (Sharma et al., (2015) Science Advances 1:e1500845). This is relevant to tumors because the PTEN-Treg pathway also controls the immune response to the wave of tumor antigens released by chemotherapy.

PTEN controls a stabilizing loop in activated Tregs. PTEN-Tregs are spontaneously present at high numbers in tumors, and they are rapidly induced by dying tumor cells (Sharma et al., (2015) Science Advances 1:e1500845). These are the same highly suppressive Tregs that we have previously described when Tregs are activated in the presence of indoleamine 2,3-dioxygenase (IDO) (Sharma et al., (2013) Immunity 38: 998-1012; Sharma et al., (2007) J. Clin. Invest. 117: 2570-2582). These IDO-activated Tregs are important in tumors, and they maintain self-tolerance to apoptotic cells. Thus, mice lacking IDO (IDO1-KO mice) fail to induce PTEN-Tregs when challenged with apoptotic self cells, and both IDO1-KO and PTEN^{Treg}-KO mice have an identical lupus-prone phenotype when challenged (Ravishankar et al., (2015) Proc. Natl. Acad. Sci. USA; Ravishankar et al., (2012) Proc. Natl. Acad. Sci. USA 109: 3909-3914).

As summarized in FIG. 10, it has been shown that PTEN lies downstream of IDO and GCN2, and maintains the long-term suppressive activity of IDO-activated Tregs. PTEN is also a centrally-positioned nexus that connects multiple important signaling pathways in Tregs. It is known that PTEN lies downstream of both neuropilin-1 (Delgoffe et al., (2013) Nature 501: 252-256) and PD-1 (Francisco et al., (2009) J. Exp. Med. 206: 3015-3029), and is also linked to mTOR and FoxO3a.

In Tregs, PTEN is important because it coordinates a stable, self-perpetuating feedback loop by inhibiting Akt kinase, stabilizing FoxO3a, and up-regulating the PD-1 receptor. Together, PD-1 and FoxO3a maintain continued PTEN activation and stabilize the suppressive Treg phenotype in tumors. If this feedback loop is interrupted, then Tregs undergo inflammation-induced reprogramming into inflammatory helper-like “ex-Tregs” (Sharma et al., (2015) Science Advances 1:e1500845; Huynh et al., (2015) Nat. lmmunol. 16: 188-196; Sharma et al., (2013) Immunity 38: 998-1012; Sharma et al. (2010) Immunity 33: 942-954). Tregs require continuous PTEN signaling or they rapidly become unstable and change into inflammatory cells (Sharma et al., (2015) Science Advances 1:e1500845). It is now possible to interrupt this pathway therapeutically with profound consequences for dendritic cell maturation and immune surveillance.

Immunogenic cell death, immune surveillance and PTEN. PTEN-Tregs act very early (immediately after the cells die) to suppress the initial maturation step of the critical cross-presenting Ly6c⁺CD103⁺ dendritic cells (FIG. 11, Step 1). This initial maturation step needs to take place before the dendritic cells can then respond to the familiar mediators of classic immunogenic cell death (HMGB1, ATP, STING, etc.) (Kroemer et al., (2013) Ann. Rev. lmmunol. 31: 51-72; Woo et al., (2015) Trends lmmunol. 36: 250-256). Thus, PTEN-Tregs frequently act to mask the fact that immunogenic cell death in tumors is potentially very common and widespread.

Translating PTEN-inhibitor drugs to the clinic. Candidate PTEN-inhibitors are under development for stroke, myocardial infarction, and diabetes (Pulido R (2015) Methods (San Diego, Calif.) 77-78: 3-10; Mak and Woscholski (2015) Methods (San Diego, Calif.) 77-78C: 63-68).

A systemic inhibitor drug is not selective for Tregs, but the key difference with respect to the immune system is that only the PTEN-Tregs seem to depend on PTEN for their function. In most other immune cells during immunotherapy, PTEN is low because effector cells need PI3K signaling for activation. Comparing the Treg-specific targeted-knockout mice versus the pharmacologic PTEN inhibitor, both yielded identical mechanistic changes by all of the immunologic readouts tested, including destabilization of Tregs, induction of Ly6c⁺CD103⁺ dendritic cells, cytokine production, activation of effector T cells, and tumor regression (see Sharma et al., (2015) Science Advances 1:e1500845). Thus, the PTEN-inhibitor drug was able to recapitulate the desired on-target effects seen in the genetic knockout without causing unacceptable off-target effects. Further, intermittent pharmacologic interruption of PTEN is not thought to have any oncogenic effect (Nardella et al., (2011) Nat. Rev. Cancer 11: 503-511), and treatment with PTEN-inhibitor does not enhance the growth of tumors in the systems of the disclosure.

PTEN^{Treg}-KO mice. In all tumors tested (B16F10, EL4, LLC, plus autochthonous Tg(Grm1)Epv melanoma and MMTV-PyMT breast cancer), up to half of all Tregs in the tumor expressed PTEN and were activated via the PTEN pathway (versus less than 10% in normal Tregs). Using existing BAC-transgenic Foxp3-Cre and PTEN-floxed mice (Zhou et al., (2009) Nat. Immunol. 10: 1000-1007; Lesche et al., (2002) Genesis 32: 148-149), targeted deletion of PTEN in Tregs (PTEN^{Treg}-KO mice) were produced. Such mice were healthy when young and did not develop autoimmunity until greater than 9-12 months of age. Even in young healthy mice, however, growth of B16F10, EL4 or LLC tumors was markedly inhibited (compare FIG. 1A), and the tumors became highly immunogenic and chronically inflamed. Importantly, the usual tolerogenic dendritic cell populations (B220+ and CD8α+) were replaced by large numbers of activated Ly6c⁺CD103⁺ dendritic cells (inset FACS plots). These dendritic cells proved to be a key cell population.

Chemo-immunotherapy with PTEN-inhibitor: In the clinic, the task is more challenging than in knockout mice. The tumor is already fully established and suppressive, and the T cells in the tumor are usually exhausted or anergic. FIG. 12A showed whether treatment with a PTEN-inhibitor drug (VO-OHpic (Mak et al., (2010) J. Chem Biol. 3: 157-163), structure as shown) could overcome this pre-existing suppression and re-activate immune surveillance.

By itself, the PTEN-inhibitor had no effect on these large established tumors; neither did chemotherapy alone (B16 melanoma tumors are highly resistant to the single moderate dose of cyclophosphamide [CTX] or gemcytabine [GEM]). However, when the PTEN-inhibitor was combined with chemotherapy there was dramatic synergy (FIG. 12B). dendritic cells in the tumor became activated and inflammatory, while the intratumoral CD8⁺ T cells underwent rapid reactivation into a CD69⁺CD103⁺IFNγ⁺ phenotype closely resembling resident-memory T cells (T_{RM} cells) (Schenkel and Masopust (2014) Immunity 41: 886-897; Schenkel et al., (2013) Nat. Immunol. 14: 509-513). All anti-tumor effect of CTX+VO-OHpic was lost in Rag1-deficient mice (FIG. 12C).

Conversely, the effect of chemotherapy could be fully replaced by adoptive transfer of exogenous tumor-specific CD8⁺ T cells (e.g., pmel-1 cells) activated with vaccine. The degree of sensitivity to PTEN-inhibitor varied with the tumor type and chemotherapy drug, but reproducible synergy was seen in every tumor tested (B16F10, EL4, LLC, Tg(Grm1)Epv, MMTV-PyMT); and both CTX and GEM showed synergy.

We claim:

1. A method for enhancing a therapeutic treatment of a cancer, said method comprising the steps of:
(a) administering to a patient in need thereof a therapeutic dose of a first therapeutic agent for the treatment of a cancer in said patient; and
(b) administering to the patient a therapeutic dose of a second therapeutic agent that elevates the level of protein p53 in said patient.

2. The method of claim 1, wherein the first therapeutic agent is an immunotherapeutic agent or a cytotoxic agent.

3. The method of claim 1, wherein the second therapeutic agent generates in a tumor a population of dendritic cells expressing at least one of Batf3, IRF5, CD103, and XCR1.

4. The method of claim 2, wherein the second therapeutic agent suppresses an autoimmune response to non-cancerous tissue in the patient generated by the immunotherapeutic agent.

5. The method of claim 2, wherein the first therapeutic agent is an immunotherapeutic agent and the second therapeutic agent enhances the immunotherapeutic response directed against a tumor in the patient.

6. The method of claim 1, further comprising administering to the patient a therapeutic dose of a PTEN phosphatase inhibitor.

7. The method of claim 1, wherein the second therapeutic agent is a Mouse Double Minute 2 (MDM2) (E3 ubiquitin-protein ligase) MDM2-related protein homolog inhibitor.

8. The method of claim 7, wherein the MDM2-related protein inhibitor is a nutlin, a benzodiazepinedione, a sulphonamide; a chromenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, or stictic acid.

9. The method of claim 8, wherein the MDM2-related protein inhibitor is nutlin-3a.

10. The method of claim 1, wherein the first therapeutic agent is an indoleamine 2,3-dioxygenase (IDO) inhibitor.

11. The method of claim 10, wherein the indoleamine 2,3-dioxygenase (IDO) inhibitor is 1-methyl-D-tryptophan (D1MT), 1-cyclohexyl-2-(5H-imidazo[5,1-a]isoindol-5-yl)ethanol (GDC919/NLG919), or (E)-4-Amino-N′-(3-chloro-4-fluorophenyl)-N-hydroxy-1,2,5-oxadiazole-3-carboximidamide (INCB024360).

12. The method of claim 2, wherein the first therapeutic agent is an anthracene cytotoxic agent selected from the group consisting of doxorubicin, idarubicin, and mitoxantrone.

13. The method of claim 1, wherein the first and the second therapeutic agents are individually administered to the patient.

14. The method of claim 1, wherein the first and the second therapeutic agents are administered in a single formulation.

15. The method of claim 6, wherein the first and the second therapeutic agents and the PTEN phosphatase inhibitor are individually administered to the patient.

16. The method of claim 6, wherein the first and the second therapeutic agents and the PTEN phosphatase inhibitor are administered in a single formulation.

17. A composition comprising a first therapeutic agent for the treatment of a cancer in a recipient patient and a second therapeutic agent that elevates the level of protein p53 in said patient.

18. The composition of claim 17, wherein the first therapeutic agent is an immunotherapeutic agent or a cytotoxic agent.

19. The composition of claim 17 further comprising a PTEN phosphatase inhibitor.

20. The composition of claim 17 further comprising a pharmaceutically acceptable carrier.

21. The composition of claim 17 formulated for delivering to a patient in need thereof an amount of an immunotherapeutic agent effective in generating an immune response directed against a tumor in the recipient patient and an amount of the second therapeutic agent effective in enhancing the immunotherapeutic response directed against a tumor of the patient by generating a population of dendritic cells expressing at least one of Batf3, IRF5, CD103, and XCR1 in the tumor.

22. The composition of claim 21, wherein the second therapeutic agent further suppresses an autoimmune response to non-cancerous tissue in the patient generated by the immunotherapeutic agent.

23. The composition of claim 17, wherein the second therapeutic agent is a Mouse Double Minute 2 (MDM2) (E3 ubiquitin-protein ligase) MDM2-related protein homolog inhibitor.

24. The composition of claim 23, wherein the MDM2-related protein inhibitor is a nutlin, a benzodiazepinedione, a sulphonamide; a chromenotriazolopyrimidine, a morpholinone, a piperidinone, a terphenyl, a chalcone, a pyrazole, an imidazole, an imidazole-indole, an isoindolinone, a pyrrolidinone, a piperidine, a naturally derived prenylated xanthone, a stapled peptide, a benzothiazole, or stictic add.

25. The composition of claim 24, wherein the MDM2-related protein inhibitor is nutlin-3a.

26. The composition of claim 18, wherein the immunotherapeutic agent is an indoleamine 2,3-dioxygenase (IDO) inhibitor.

27. The composition of claim 26, wherein the indoleamine 2,3-dioxygenase (IDO) inhibitor is 1-methyl-D-tryptophan (D1MT), 1-cyclohexyl-2-(5H-imidazo[5,1-a]isoindol-5-yl)ethanol (GDC919/NLG919), or (E)-4-Amino-N′-(3-chloro-4-fluorophenyl)-N-hydroxy-1,2,5-oxadiazole-3-carboximidamide (INCB024360).

28. The composition of claim 18, wherein the cytotoxic agent is an anthracene selected from the group consisting of doxorubicin, idarubicin, mitoxantrone.

29. A composition comprising an immunotherapeutic agent effective in generating an immune response directed against a tumor in a recipient patient, a therapeutic agent that elevates the level of protein p53 in a recipient patient, wherein said therapeutic agent is nutlin-3a, and a pharmaceutically acceptable carrier.

30. The composition of claim 29, further comprising at least one of an IDO-inhibitor and a cytotoxic agent.

31. A kit comprising an first therapeutic agent directed against a tumor in a recipient patient, a second therapeutic agent that elevates the level of protein p53 in a recipient patient, and a pharmaceutically acceptable carrier, wherein the first therapeutic agent, the second therapeutic agent, and the pharmaceutically acceptable carrier are packaged individually or in any combination, and instructions for the use of the packaged agents and carrier to prepare an effective dose of each agent for administration individually or in combination to a patient in need thereof.
